(19) United States
(12) Patent Application Publication
(10) Pub. No.: US 2022/0024991 A1
(43) Pub. Date: Jan. 27, 2022

(54) ENGINEERED FLAGELLIN-DERIVED COMPOSITIONS AND USES

(71) Applicant: Genome Protection, Inc., Buffalo, NY (US)

(72) Inventors: Vadim METT, Buffalo, NY (US); Andrei GUDKOV, Buffalo, NY (US)

(21) Appl. No.: 17/299,373

(22) PCT Filed: Dec. 6, 2019
(86) PCT No.: PCT/US19/64954

§ 371 Date: Jun. 3, 2021

Related U.S. Application Data
(60) Provisional application No. 62/776,507, filed on Dec. 7, 2018.

(51) Int. Cl. C07K 14/195; A61K 38/16; A61K 45/06

(52) U.S. Cl. CPC C07K 14/195; A61K 45/06; A61K 38/164

(57) ABSTRACT

The present invention provides improved pharmacologically optimized and deimmunized flagellin variants and methods of use that exhibit reduced immunogenicity and reduced inflammasome response while still retaining the ability to activate TLR5 signaling.

### PRIORITY

This application claims the benefit of, and priority to, U.S. Provisional Application No. 62/776,507, filed Dec. 7, 2018, the content of which is herein incorporated by reference in its entirety.

### FIELD OF THE INVENTION

This invention relates to engineered flagellin variants, compositions and uses thereof.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GPI-020PC_ST25.txt; date recorded: Dec. 6, 2019; file size: 90,275 bytes).

### BACKGROUND

Hyperactivity of inflammatory signaling complexes, known as “the inflammasome,” has been linked to a variety of inflammatory diseases. The inflammasome paradigm is most comprehensively illustrated by the NLRC4 inflammasome, which includes a trigger (e.g. cytosolic flagellin), sensor (NAIP), nucleator (NLRC4), adaptor (ASC), and effector (CASP1). For example, extracellular flagellin can activate the cytoplasmic NLRC4 inflammasome due to internalization of flagellin-TLR5 complexes. Once assembled, the inflammasome initiates a pro-inflammatory cascade involving caspase-1 activation and, subsequently, cleaving the inactive precursors of IL-1β and IL-18 into bioactive, pro-inflammatory cytokines.

Toll-like receptors (TLRs) are type I membrane glycoproteins that are key receptors in innate immunity. The 10 TLRs known in humans recognize different microbial antigens, and when activated by ligand binding, mediate rapid production of cytokines and chemokines. In addition to their role in host defense, TLRs play a role in cancer progression and development and cell protection. One such example is the binding of flagellin to TLR5, which initiates a cascade of pro-inflammatory molecules.

TLR5 agonists derived from flagellin have been developed as therapies for various diseases. However, these molecules may suffer from specific limitations, including for example, unsatisfactory binding and signaling, and the activation of inflammatory cytokines through the inflammasome pathway, thus limiting therapeutic effect. Intrinsically immunogenic flagellin variants may possess disadvantageous inflammasome activation, antigenicity and immunogenicity, and therefore warrant improvement.

### SUMMARY OF THE INVENTION

Accordingly, the present invention provides improved flagellin variants and methods of use that overcome limitations observed among this group of biologics, such as those related to immunogenicity and inflammasome activation.

The present invention is based, in part, on the discovery that mutated variants of flagellin can exhibit reduced immunogenicity and reduced inflammasome response while still retaining the ability to activate TLR5 signaling.

In one aspect, the invention provides a flagellin variant that retains the ability to activate TLR5 signaling. In some embodiments, the flagellin variant induces NF-κB promoter. In some embodiments, the flagellin variant exhibits similar or higher NF-κB signaling activity relative to NF-κB signaling activity exhibited by entolimod or other flagellin derivatives. In some embodiments, the flagellin variant retains radioprotective and/or radiomitigation activity. In further embodiments, the flagellin variant exhibits similar or better radioprotective and/or radiomitigation activity relative to radioprotective and/or radiomitigation activity exhibited by entolimod or other flagellin derivatives. In a further embodiment, the flagellin variant comprises mutations that reduce inflammasome activation of the construct, as compared to entolimod. Specifically, in some embodiments, the flagellin variant exhibits lower inflammasome activation relative to inflammasome activation exhibited by entolimod or other flagellin derivatives. In another embodiment, the flagellin variant comprises mutations that decrease T cell immunogenicity of the construct, as compared to entolimod. In a further embodiment, the flagellin variant exhibits reduced sensitivity to B cell neutralizing antibodies, as compared to entolimod. In another embodiment, the flagellin variant demonstrates improved resistance to neutralizing B cell antibodies (e.g., substantially free of neutralizing antibodies), as compared to entolimod. In yet a further embodiment, the flagellin variant activates TLR5 signaling at a level the same as or similar to that of entolimod. In a further embodiment, the flagellin variant demonstrates the same as or a similar or an improved pharmacokinetics profile compared with entolimod. In yet a further embodiment, the flagellin variant demonstrates increased or similar retention in the host as compared to retention of entolimod. In embodiments, administration of a flagellin variant of the present invention to a subject results in a substantially increased duration of bioavailability of the flagellin variant.

In some embodiments, the flagellin variant is derived from 33MX (SEQ ID NO: 1). In some embodiments, the flagellin variant is derived from entolimod/CBLB502 (SEQ ID NO: 3). In some embodiments, the flagellin variant comprises at least one amino acid substitution in one or more epitopes. In some embodiments, the flagellin variant comprises at least one amino acid substitution in at least two different epitopes. In some embodiments, the flagellin variant comprises at least one deletion in one or more epitopes. In a further embodiment, the flagellin variant comprises an amino acid substitution and/or deletion in one or more of epitope 1, epitope 2, and epitope 3. In a further embodiment, the flagellin variant retains NF-kB signaling activity.

In some aspects, the present invention contemplates a flagellin variant comprising an amino acid sequence having at least 90% sequence identity with SEQ ID NO: 1 and (i) a substitution mutation at a position corresponding to one or more of I18, F22, T23, S24, and K27, and (ii) a substitution mutation at a position corresponding to one or more of I215, L216, Q217, T221, and V223, wherein the substituted amino acid residue is any naturally-occurring amino acid, and wherein the flagellin variant retains NF-kB signaling activity. In some embodiments, the flagellin variant comprises at least one substitution or deletion mutation selected from amino acid residue position(s) corresponding to I18, F22, T23, S24, K27, I215, L216, Q217, T221, and V223. In some embodiments, the flagellin variant comprises at least one substitution or deletion mutation selected from amino acid residue position(s) corresponding to I18, F22, T23, S24, and K27, and at least one substitution or deletion mutation selected from amino acid residue position(s) corresponding to I215, L216, Q217, T221, and V223. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant comprises 118A, F22A, Q217D, and V223T. In a further embodiment, the flagellin variant retains NF-kB signaling activity. In a further embodiment, the flagellin variant is 491TEMX (SEQ ID NO: 2, optionally without a terminal Histidine tag, e.g. SEQ ID NO: 5).

In some embodiments, the flagellin variant comprises an amino acid sequence having at least 60%, 65%, 70%, 75%, 80%, 85%, 90%, or 95% sequence identity with SEQ ID NO: 1. In a further embodiment, the flagellin variant is 491TEMX (SEQ ID NO: 2, optionally without a terminal Histidine tag, e.g. SEQ ID NO: 5).

In some embodiments, the flagellin variant comprises a tag. In yet a further embodiment, the tag is attached to the N-terminus of the flagellin variant. In yet another embodiment, the tag is attached to the C-terminus of the flagellin variant.

The present invention provides for a flagellin variant that can induce NF-κB promoter. In some embodiments, the flagellin variant induces expression of one or more of cytokines. In a further embodiment, the cytokines are selected from G-CSF, IL-6, IL-12, keratinocyte chemoattractant (KC), IL-10, MCP-1, TNF-α, MIG, and MIP-2.

In one aspect, the invention provides a polynucleotide comprising a polynucleotide sequence encoding the flagellin variant of of the invention.

In one aspect, the invention provides a pharmaceutical composition comprising the flagellin variant of the invention with a pharmaceutically accepted carrier.

In one aspect, the invention provides a method of stimulating TLR5 signaling comprising administering a flagellin variant of the invention to a subject in need thereof. In some embodiments, the subject has cancer. In further embodiments, the tumor expresses TLR5. In further embodiments, the tumor does not express TLR5. In some embodiments, the cancer is selected from breast cancer, lung cancer, colon cancer, kidney cancer, liver cancer, ovarian cancer, prostate cancer, testicular cancer, genitourinary tract cancer, lymphatic system cancer, rectal cancer, pancreatic cancer, esophageal cancer, stomach cancer, cervical cancer, thyroid cancer, skin cancer, leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, and Burkett's lymphoma, acute and chronic myelogenous leukemias, myelodysplastic syndrome, myeloid leukemia, promyelocytic leukemia, astrocytoma, neuroblastoma, glioma, schwannomas, fibrosarcoma, rhabdomyoscarcoma, osteosarcoma, xenoderma pigmentosum, keratoactanthoma, seminoma, thyroid follicular cancer, teratocarcinoma, and cancers of the gastrointestinal tract or the abdominopelvic cavity.

In some embodiments, the subject suffers from radiation-induced damage. In a further embodiment, the subject has been subjected to a lethal dose of radiation. In some embodiments, the subject is undergoing radiation treatment. In some embodiments, the flagellin variant is administered prior to exposure to radiation. In some embodiments, the flagellin variant is administered during exposure to radiation. In some embodiments, the flagellin variant is administered after exposure to radiation.

In various embodiments, the flagellin variant is administered in conjunction with other therapeutics and/or treatments. In some embodiments, the flagellin variant is administered in conjunction with chemotherapy. In further embodiments, the flagellin variant is administered with radiation treatment. In some embodiments, the flagellin variant is administered in conjunction with an antioxidant. In a further embodiment, the flagellin variant is administered in conjunction with amifostine and/or vitamin E. In some embodiments, the flagellin variant is administered in conjunction with one or more checkpoint inhibitors. In a further embodiment, the one or more checkpoint inhibitors is selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4). In some embodiments, the flagellin variant is administered prior to administration of other therapeutics and/or treatments. In further embodiments, the flagellin variant is administered at the same time as other therapeutics and/or treatments. In yet further embodiments, the flagellin variant is administered after administration of other therapeutics and/or treatments.

In one aspect, the present invention provides for an engineered flagellin variant comprising an amino acid sequence having at least 90%, at least 91%, at least 92%, at least 93%, at least 94%, at least 95%, at least 96%, at least 97%, at least 98%, or at least 99% sequence identity with SEQ ID NO: 2, optionally without a terminal histidine tag (e.g., SEQ ID NO: 5). In another aspect, the invention provides for an engineered flagellin variant comprising a polypeptide having an amino acid sequence that is SEQ ID NO: 2, optionally without a terminal histidine tag (e.g., SEQ ID NO: 5).

In one aspect, the present invention provides for an engineered flagellin variant comprising an amino acid sequence having at least 90%, at least 91%, at least 92%, at least 93%, at least 94%, at least 95%, at least 96%, at least 97%, at least 98%, or at least 99% sequence identity with SEQ ID NO: 2, wherein the amino acid sequence of SEQ ID NO: 2 does not comprise a terminal histidine tag sequence. In another aspect, the invention provides for an engineered flagellin variant comprising an amino acid sequence that is SEQ ID NO: 2, wherein the amino acid sequence of SEQ ID NO: 2 does not comprise a terminal histidine tag sequence. In further embodiments, the amino acid sequence of the terminal histidine tag is SEQ ID NO: 5.

In one aspect, the invention provides a method of treating cancer comprising administering a flagellin variant of the invention to a subject in need thereof.

In one aspect, the invention provides a method of treating radiation-induced damage comprising administering a flagellin variant of the invention to a subject in need thereof.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1 depicts the amino acid sequence of 491TEMX (a.k.a. SE-2/GP532), which is SEQ ID NO: 2,

FIG. 2 shows a comparison of the frequency of donor allotypes expressed in the study cohort (n=50) with the European/North American and world population. With respect to each set of histograms, from left to right, the first bar respresents CBLO1 donors; the second bar represents European and North American donors; and the third and last bar represents world population donors.

FIG. 3 depicts CD4⁺ T cell epitope map using peptides tested against PBMC from 50 healthy donors. The non-adjusted and adjusted proliferation assay data for the 80 test peptides and controls C3, C32 and KLH. Peptides inducing positive (SI≥2.00, p<0.05) T cell proliferation responses at a frequency above the background response threshold (indicated by the red dotted line) contain T cell epitopes. KLH induced positive responses (SI≥2.00, p<0.05) in 92% of donors in both the non-adjusted and adjusted data sets. With respect to each set of histograms, the left bar represents non-adjusted proliferation assay data, and the right bar represents adjusted proliferation assay data.

FIG. 4A-B shows reducing SDS-PAGE of the samples and a reference antibody. The samples and a reference antibody were loaded at (FIG. 4A) 0.1 μg and (FIG. 4B) 1 μg onto NuPage 4-12% Bis-Tris gels (ThermoFisher Scientific) and run at 200 V for 30 min. Size marker is PageRuler broad range unstained protein ladder (ThermoFisher Scientific). Gels were stained with a Pierce Silver Stain Kit (ThermoFisher Scientific).

FIG. 5 depicts a summary of healthy donor T cell proliferation and IL-2 ELISpot responses to Sample 1, entolimod/CBLB502, and Sample 2, 491TEMX (a.k.a. SE-2 and GP532). Positive T cell responses for proliferation (SI≥2.00, p<0.05) (“P”), and IL-2 (SI≥2.00, p<0.05) ELISpot (“E”) after 7 days' culture are shown. The frequency of positive responses for proliferation and IL-2 ELISpot assays are shown as a percentage at the bottom of the columns. Correlation is expressed as the percentage of proliferation responses that were also positive in the IL-2 ELISpot assay.

FIG. 6A-C shows healthy donor T cell proliferation responses to: FIG. 6A, sample 1 (entolimod, a.k.a. CBLB502); FIG. 6B, sample 2 (491TEMX, a.k.a. SE-2 and GP532); and FIG. 6C, KLH (control). CD4⁺ T cells were incubated with autologous mature DC loaded with the samples and assessed for proliferation after 7 days' incubation. T cell responses with an SI≥1.90 (indicated by red dotted line) that were significant (p<0.05) using an unpaired, two sample Student's t-test were considered positive.

FIG. 7A-C depicts healthy donor T cell IL-2 secretion response to: FIG. 7A, sample 1 (entolimod, a.k.a. CBLB502); FIG. 7B, sample 2 (491TEMX, a.k.a. SE-2 and GP532); and FIG. 7C, KLH (control). CD4⁺ T cells were incubated with autologous mature DC loaded with the samples and assessed for IL-2 secretion after 7 days' incubation. T cell responses with an SI≥1.90 (indicated by red dotted line) that were significant (p<0.05) using an unpaired, two sample Student's t-test were considered positive.

FIG. 8 depicts NF-κB signaling induced in 293-hTLR5-LacZ reporter cells by variant flagellin variants, namely 33MX, 33TX2 (a.k.a. SE-1), and 491TEMX (a.k.a. SE-2 and GP532).

FIG. 9 depicts inflammasome activity induced in THP1-NLRC4 cells (e.g., IL-1β production) by variant flagellin variants, where IL-1β represents an inflammasome marker. The flagellin variants shown are CBLB502 (a.k.a. entolimod), 33MX, 33TX2 (a.k.a. SE-1), and 491TEMX (a.k.a. SE-2 and GP532).

FIG. 10 shows a pharmacokinetics profile of the flagellin variants SE-1 (a.k.a. 33TX2), SE-2 (a.k.a. 491TEMX and GP532), and entolimod (a.k.a. CBLB502) after each was injected into mice. Measurement of the resultant concentration in ng/ml over the course of 24 hours is shown, demonstrating that SE-2 performed better or equal to entolimod.

FIG. 11 depicts measurement of the pharmacodynamics marker cytokine G-CSF over the course of 24 hours after injection of SE-1 (a.k.a. 33TX2), SE-2 (a.k.a. 491TEMX and GP532), and entolimod (a.k.a. CBLB502).

FIG. 12 depicts measurement of the pharmacodynamics marker cytokine IL-6 over the course of 24 hours after injection of SE-1 (a.k.a. 33TX2), SE-2 (a.k.a. 491TEMX and GP532), and entolimod (a.k.a. CBLB502).

FIG. 13 depicts measurement of the inflammasome marker IL-18 over the course of 24 hours after injection of SE-1 (a.k.a. 33TX2), SE-2 (a.k.a. 491TEMX and GP532), and entolimod (a.k.a. CBLB502).

FIG. 14 shows measurement of nitric oxide over the course of 24 hours after injection of SE-1 (a.k.a. 33TX2), SE-2 (a.k.a. 491TEMX and GP532), and entolimod (a.k.a. CBLB502).

FIG. 15 depicts a dose study of entolimod (a.k.a. CBLB502) and SE-2 (a.k.a. 491TEMX and GP532), respectively, where the doses were 4 μg/kg, 6 μg/kg, 8 μg/kg, 16 μg/kg, 32 μg/kg, and 64 μg/kg, and PBS-Tween was used as a control. Radioprotectivity was measured by percent survival of mice over the course of the 27-day study.

FIG. 16 shows radioprotectivity as measured by percent survival of mice over the course of 60 days after total body irradiation. Before total body irradiation, mice were subjected to human serum transfer with serum containing neutralizing antibodies or normal serum, followed by injection with entolimod (a.k.a. CBLB502), SE-2 (a.k.a. 491TEMX and GP532), or PBS. As measured at the 60 day enpoint, from top to bottom, the top line represents entolimod+normal serum; the second line represents SE-2+normal serum; the third line represents SE-2+neutralizing serum; the fourth line represents entolimod+neutralizing serum; and the bottom line represents the PBS control.

FIG. 17 depicts the results of a study conducted measuring the combination tumor treatment with SE-2 (a.k.a. 491TEMX and GP532) and checkpoint inhibitors. An EMT6 mouse model of triple-negative breast cancer was used, where treatment began with administration of checkpoint inhibitors, followed by administration of entolimod or SE-2. Specifically, the mice were given a dose of α-PD1 on day 7, followed by a dose of α-CTLA4 on day 9. On days 10 and 11, doses of entolimod and SE-2 were administered. The results show that the combination of administration of checkpoint inhibitors followed by administration of SE-2 exhibited faster tumor regression than administration of entolimod with checkpoint inhibitors. At day 21, from top to bottom, the top line represents isotypes+Vehicle; the second line represents isotypes+entolimod; the third line represents isotypes+SE-2; the fourth line represents α-CTLA4+α-PD-1+entolimod; the fifth line represents α-CTLA4+α-PD-1+Vehicle; and the sixth (i.e., bottom) line represents α-CTLA4+α-PD-1+SE-2.

FIG. 18 depicts histological analysis of mouse liver hepatocytes and shows NF-κB activation by GP532 (a/k/a 491TEMX) and entolimod.

FIG. 19 shows in vivo imaging of signaling activity in NF-κB-luciferase reporter mice upon transfusion of neutralizing or non-neutralizing (control) human serum followed by subcutaneous injection of Entolimod or GP532

FIG. 20 depicts the results of a radiomitigation study by assessing percent survivial over a period of 60 days after Balb/c mice were administered vehicle, entolimod or GP532 and were then subjected to lethal total body irradiation. As measured at the 20-day point, from top to bottom, the top line represents entolimod, the second line represents GP532, and the third line represents the PBS vehicle control.

FIG. 21A-G depicts histology scores of mouse areas—skin (FIG. 21A), vermillion (FIG. 21B), mouth (FIG. 21C), lymph nodes (FIG. 21D), submandibular (FIG. 21E), sling muc (FIG. 21F), and parotid (FIG. 21G)—after having been administered vehicle, entolimod or GP532 and then subjected to total body irradiation.

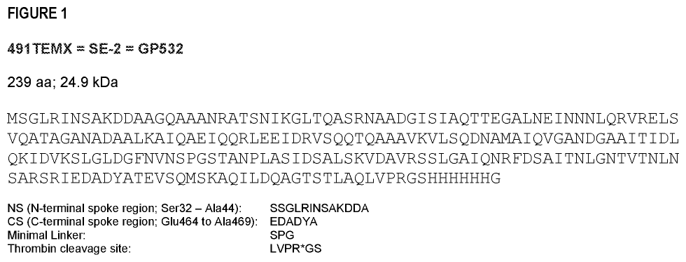

### DETAILED DESCRIPTION OF THE INVENTION

The present invention is based, in part, on the discovery of certain mutations of flagellin that improve pharmacologically relevant properties of this biologic and related agents. Such mutations yield various flagellin variants that, by way of non-limiting example, have reduced immunogenicity and reduced inflammasome activity, relative to those without the mutations. The flagellin variants retain their TLR5 signaling abilities and radioprotective and/or radiomitigative abilities at levels the same as, or similar to, that of entolimod and other flagellin variants.

### Flagellin Variants

The present invention is based, in part, of the discovery that mutated flagellin variants can exhibit reduced immunogenicity and reduced inflammasome activation while still retaining the ability to active TLR5 signaling at levels the same as, or similar to, that of entolimod or other flagellin variants. The reduced immunogenicity allows the flagellin variant to persist in the host longer and provides for a multi-use protein due to reduced induction of neutralizing antibodies (e.g., substantially free of neutralizing antibodies) as compared to entolimod or other flagellin variants, and the reduced inflammasome activation allows for more desirable therapeutic applications of the mutatated flagellin variant of the present invention.

In various embodiments, the present invention provides flagellin variants. In some embodiments, the present invention provides for flagellin variants that have (1) improved pharmacological properties, including reduced antigenicity, immunogenicity, and inflammasome activation, which, for example, allow for use in wide variety of disease states and patient types and/or (2) improved functional properties which, for example, allow for improved medical effects.

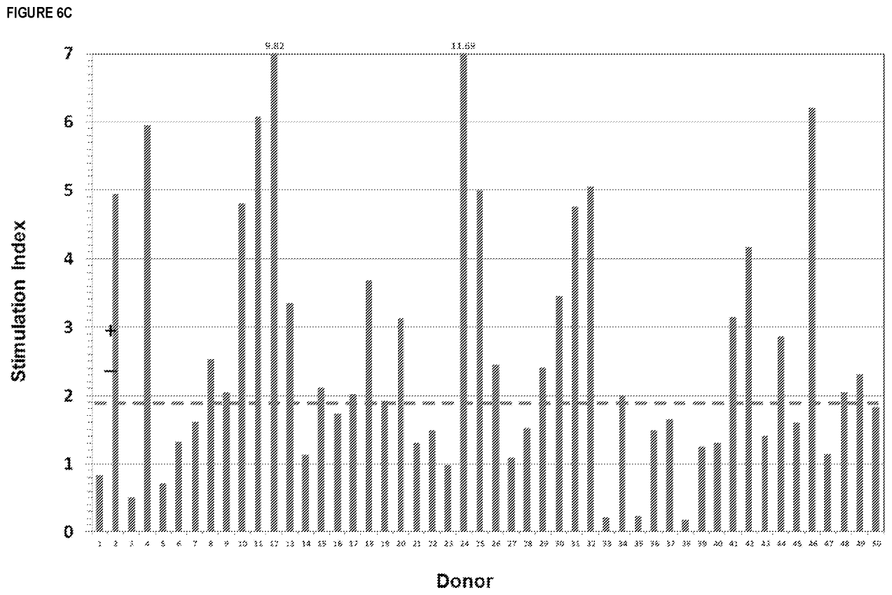

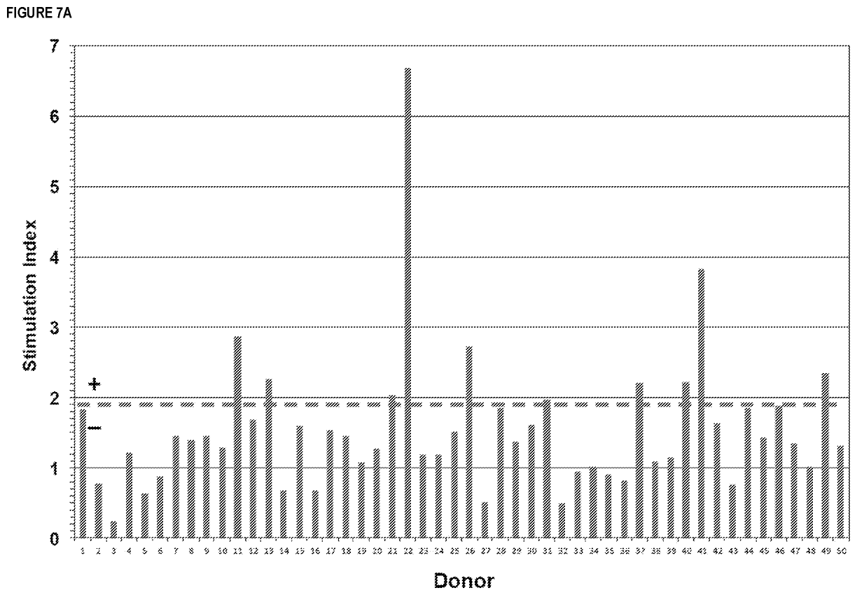

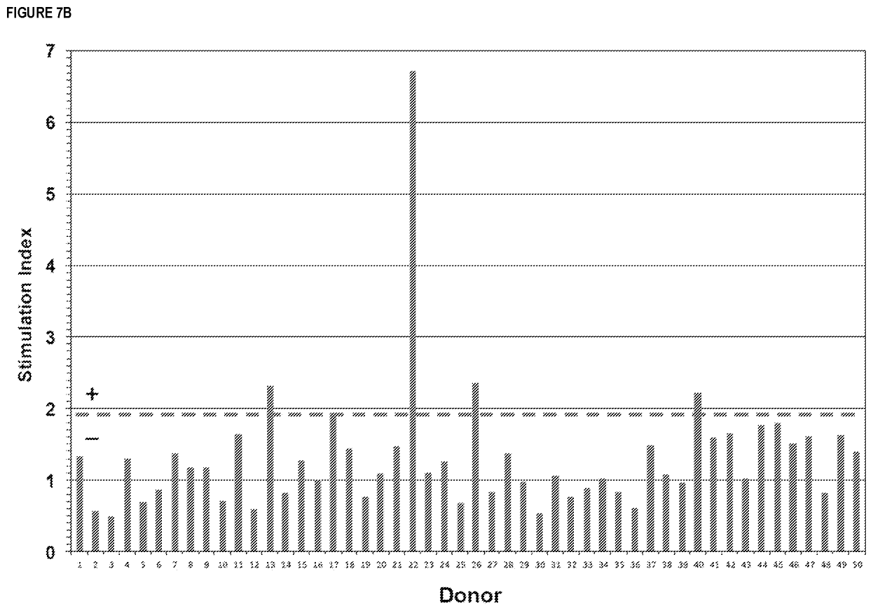

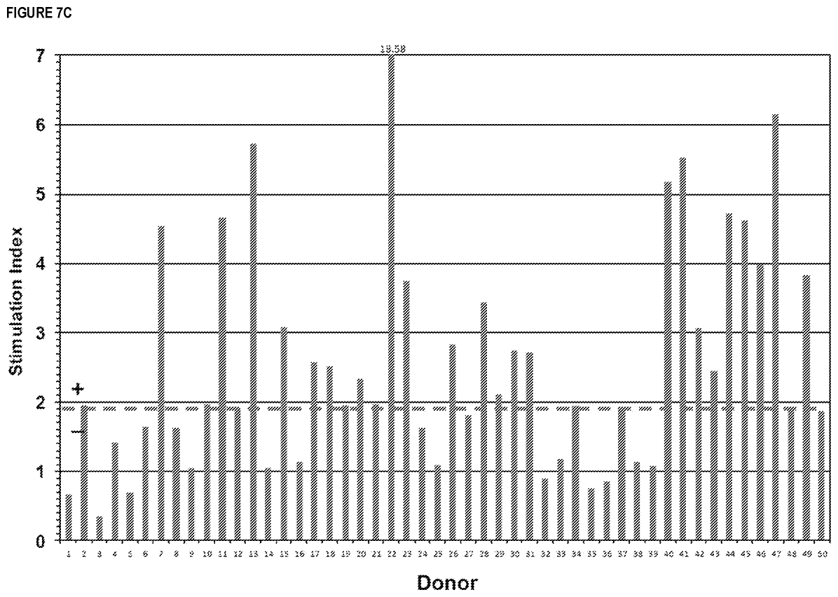

The flagellin variant of the present invention may be a flagellin-related polypeptide. The flagellin variants may be from various sources, including a variety of Gram-positive and Gram-negative bacterial species. In some embodiments, the flagellin variants may have an amino acid sequence that is derived from any of the flagellins from bacterial species that are depicted in FIG. 7 of U.S. Patent Publication No. 2003/0044429, the contents of which are incorporated herein by reference in their entirety. The flagellin variants may have nucleotide sequences related to those encoding the flagellin polypeptides listed in FIG. 7 of U.S. 2003/0044429, which are publicly available at sources including the NCBI Genbank database.

The flagellin variants may be the major component of bacterial flagellum. The flagellin variants may be composed of one, or two, or three, or four, or five T cell epitopes that are characterized by positive T cell responses to various peptide regions. In some embodiments, the flagellin variant is composed of three T cell epitopes. In further embodiments, the T cell epitopes comprise low frequency positive T cell proliferation responses. In various embodiments, the T cell epitope strength is low, or moderate, or strong.

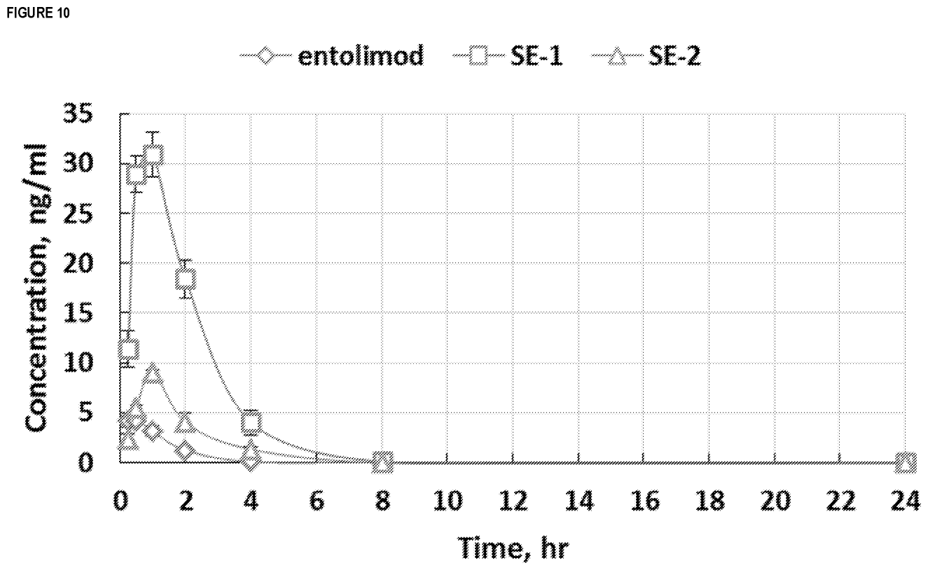

The flagellin variants may be the major component of bacterial flagellum. The flagellin variants may be composed of one, or two, or three, or four, or five, or six, or seven domains or fragments thereof (see, e.g. FIG. 10 of U.S. Pat. No. 8,324,163, the contents of which are incorporated herein by reference in their entirety). The domains may be selected from ND0, ND1, ND2, D3, CD2, CD1, and CD0. Domains 0 (D0), 1 (D1), and 2 (D2) may be discontinuous and may be formed when residues in the amino terminus and carboxy terminus are juxtaposed by the formation of a hairpin structure. The amino and carboxy terminus comprising the D1 and D2 domains may be most conserved, whereas the middle hypervariable domain (D3) may be highly variable. The non-conserved D3 domain may be on the surface of the flagellar filament and may contain the major antigenic epitopes. The potent proinflammatory activity of flagellin may reside in the highly conserved ND1, ND2, CD1, and CD2 regions.

The flagellin variants may be from a species of Salmonella, representative examples of which are S. typhimurium and S. dublin (encoded by GenBank Accession Number M84972). The flagellin variant may be a fragment, variant, analog, homolog, or derivative of wild type flagellin (SEQ ID NO: 4), or combination thereof. A fragment, variant, analog, homolog, or derivative of flagellin may be obtained by rational-based design based on the domain structure of flagellin and the conserved structure recognized by TLR5.

The flagellin variants may be related to a flagellin polypeptide from any Gram-positive or Gram-negative bacterial species including, but not limited to, the flagellin polypeptides disclosed in U.S. Pat. Pub. 2003/000044429, the contents of which are incorporated herein, and the flagellin peptides corresponding to the Accession numbers listed in the BLAST results shown in FIG. 7 (panels A-F) of U.S. Patent Pub. 2003/000044429, or variants thereof.

Flagellin and previously described variants suffer from high antigenicity and immunogenicity in large part, without wishing to be bound by theory, because they are intrinsically immunogenic bacterial proteins (e.g. flagellin or “FliC”). A practical limitation in preexisting flagellin constructs is that many subjects have high titers of pre-existing antibodies capable of neutralizing the TLR5-stimulating activity of these constructs. These individuals would be desensitized (or completely resistant) to flagellin-derived treatment, sometimes even in case of single-injections and, without wishing to be bound by theory, more likely upon recurrent treatment. Moreover, the titer of such pre-existing antibodies, even if initially present at lower levels, may be rapidly boosted by a single flagellin-derived injection thereby compromising even a larger group of individuals for the purpose of multi-dose regimen as projected for medical applications. The widespread preexistence of anti-FliC antibodies (including neutralizing Abs) in a population likely reflects humanity's life-long exposure to numerous species of flagellated enterobacteria (e.g. Salmonella spp., E. coli) colonizing (and infecting) the human body. In some embodiments, the presently described flagellin variants comprise alterations of epitopes for various antibodies that neutralize flagellin activity.

Furthermore, flagellin and previously described variants suffer from inflammasome activation. Without wishing to be bound by any one theory, it is thought that extracellular flagellin is able to activate the cytoplasmic NLRC4 inflammasome due to internalization of flagellin-TLR5 complexes. The NLRC4 inflammasome is one of a number of cytoplasmic multi-molecule complexes that is assembled following activation of its pattern recognition receptor (PRR) component by a microbial entity. In the case of NLRC4, the cytoplasmic Nod-like receptor (NLR) is activated by bacterial flagellin, which is also an agonist of Toll-like receptor 5 (TLR5) on the cell membrane. It is assumed that extracellular flagellin is able to activate the cytoplasmic NLRC4 inflammasome due to internalization of flagellin-TLR5 complexes. Once assembled, the inflammasome initiates a pro-inflammatory cascade involving caspase-1 activation and, subsequently, processing of pro-IL-1β to mature IL-1β, which is major pro-inflammatory cytokine. As a result of such inflammasome activation, subjects may experience undesirable side effects making therapeutic applications more difficult.

In some embodiments, the flagellin variant comprises mutations in epitopes recognized by neutralizing anti-CBLB502 antibodies. The flagellin variant may comprise one or more mutations in the epitopes recognized by neutralizing anti-CBLB502 antibodies which inhibit or abrogate the ability of the antibodies to neutralize the composition. In some embodiments, the flagellin variant induces a response in a subject that is substantially free of neutralizing antibodies. In some embodiments, the flagellin variant comprises mutations that inhibit inflammasome activation. In yet a further embodiment, the flagellin variant comprises a truncation and mutations in one or more epitopes.

In some embodiments, the present invention relates to the development of a minimal functional core of a flagellin, for example, deleting residues relative to the already shortened entolimodrCBLB502″ molecule. In some embodiments, the present invention relates to the development of a flagellin variant that has altered amino acid identity relative to entolimod/CBLB502, including deletions, additions and substitutions that provide for improved activity. In some embodiments, the flagellin variant is derived from entolimod/CBLB502 (SEQ ID NO: 3). In some embodiments, the flagellin variant comprises comprises an amino acid sequence having at least 60%, at least 65%, at least 70%, at least 75%, at least 80%, at least 85%, at least 90%, at least 92%, at least 93%, at least 94%, at least 95%, at least 96%, at least 97%, or at least 98% sequence identity with SEQ ID NO: 3. In some embodiments, the flagellin variant comprises at least one amino acid substitution in one or more epitopes. In some embodiments, the flagellin variant comprises at least one amino acid substitution in at least two different epitopes. In some embodiments, the flagellin variant comprises at least one deletion in one or more epitopes. In a further embodiment, the flagellin variant comprises an amino acid substitution and/or deletion in one or more of epitope 1, epitope 2, and epitope 3. In a further embodiment, the flagellin variant retains NF-kB signaling activity.

In some embodiments, the present invention relates to the development of a minimal functional core of a flagellin, for example, deleting residues relative to the already shortened “33MX” molecule. In some embodiments, the present invention relates to the development of a flagellin variant that has altered amino acid identity relative to 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 60%, at least 65%, at least 70%, at least 75%, at least 80%, at least 85%, at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In further embodiments, the flagellin variant comprises at least one amino acid substitution in one or more epitopes. In yet further embodiments, the flagellin variant comprises at least one amino acid substitution in at least two different epitopes. In further embodiments, the flagellin variant comprises at least one deletion in one or more epitopes. In a further embodiment, the flagellin variant comprises an amino acid substitution and/or deletion in one or more of epitope 1, epitope 2, and epitope 3. In a further embodiment, the flagellin variant retains NF-kB signaling activity.

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In further embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27, and at least one substitution or deletion mutation selected from amino acid residue position(s) corresponding to I215, L216, Q217, T221, and V223. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant comprises 118A, F22A, Q217D, and V223T. In a further embodiment, the flagellin variant retains NF-kB signaling activity. In a further embodiment, the flagellin variant is 491TEMX (SEQ ID NO: 2, optionally without a terminal Histidine tag).

In some embodiments, the present invention contemplates a flagellin variant comprising an amino acid sequence having at least 90% sequence identity with SEQ ID NO: 1 and (i) a substitution mutation at a position corresponding to one or more of I18, F22, T23, S24, and K27, and (ii) a substitution mutation at a position corresponding to one or more of I215, L216, Q217, T221, and V223, wherein the substituted amino acid residue is any naturally-occurring amino acid, and wherein the flagellin variant retains NF-kB signaling activity.

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22A and T23D. In a further embodiment, the flagellin variant is TEM1-AD (SEQ ID NO: 7, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22S and T23D. In a further embodiment, the flagellin variant is TEM1-SD (SEQ ID NO: 8, optinally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22T and T23D. In a further embodiment, the flagellin variant is TEM1-TD (SEQ ID NO: 9, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises T23D and S24D. In a further embodiment, the flagellin variant is TEM1-DD (SEQ ID NO: 10, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises 118A. In a further embodiment, the flagellin variant is TEM1-49A (SEQ ID NO: 11, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22A. In a further embodiment, the flagellin variant is TEM1-53A (SEQ ID NO: 12, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises T23D. In a further embodiment, the flagellin variant is TEM1-54D (SEQ ID NO: 13, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises I18E. In a further embodiment, the flagellin variant is TEM1-49E (SEQ ID NO: 14, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises 118T. In a further embodiment, the flagellin variant is TEM1-49T (SEQ ID NO: 15, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises K27E. In a further embodiment, the flagellin variant is TEM1-58E (SEQ ID NO: 16, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (1), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises 1215A. In a further embodiment, the flagellin variant is TEM2-480A (SEQ ID NO: 23, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises L216A. In a further embodiment, the flagellin variant is TEM2-481A (SEQ ID NO: 24, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (1), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises V223T. In a further embodiment, the flagellin variant is TEM2-488T (SEQ ID NO: 25, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (1), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises Q217D. In a further embodiment, the flagellin variant is TEM2-482D (SEQ ID NO: 28, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX (SEQ ID NO: 1, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 1, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises T221D. In a further embodiment, the flagellin variant is TEM2-486D (SEQ ID NO: 29, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In further embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27, and at least one substitution or deletion mutation selected from amino acid residue position(s) corresponding to I215, L216, Q217, T221, and V223. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (1), methionine (M), and valine (V), or a hydrophobic, aromatic amino acid residue, selected from phenylalanine (F), tryptophan (W), and tyrosine (Y). In a further embodiment, the flagellin variant comprises 118A, F22A, Q217D, and V223T. In a further embodiment, the flagellin variant retains NF-kB signaling activity. In a further embodiment, the flagellin variant is 491TEMX (SEQ ID NO: 2, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22A and T23D. In a further embodiment, the flagellin variant is TEM1-AD (SEQ ID NO: 7, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22S and T23D. In a further embodiment, the flagellin variant is TEM1-SD (SEQ ID NO: 8, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22T and T23D. In a further embodiment, the flagellin variant is TEM1-TD (SEQ ID NO: 9, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises T23D and S24D. In a further embodiment, the flagellin variant is TEM1-DD (SEQ ID NO: 10, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises 118A. In a further embodiment, the flagellin variant is TEM1-49A (SEQ ID NO: 11, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises F22A. In a further embodiment, the flagellin variant is TEM1-53A (SEQ ID NO: 12, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises T23D. In a further embodiment, the flagellin variant is TEM1-54D (SEQ ID NO: 13, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises I18E. In a further embodiment, the flagellin variant is TEM1-49E (SEQ ID NO: 14, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises 118T. In a further embodiment, the flagellin variant is TEM1-49T (SEQ ID NO: 15, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I18, F22, T23, S24, and K27. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises K27E. In a further embodiment, the flagellin variant is TEM1-58E (SEQ ID NO: 16, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises 1215A. In a further embodiment, the flagellin variant is TEM2-480A (SEQ ID NO: 23, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises L216A. In a further embodiment, the flagellin variant is TEM2-481A (SEQ ID NO: 24, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises V2231. In a further embodiment, the flagellin variant is TEM2-488T (SEQ ID NO: 25, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises Q217D. In a further embodiment, the flagellin variant is TEM2-482D (SEQ ID NO: 28, optionally without a terminal Histidine tag).

In some embodiments, the present invention relates to the development of a flagellin variant that has altered amino acid identity relative to entolimod or other flagellin variants, such as 33MX, including deletions, additions and substitutions, that provide for improved activity. In some embodiments, the flagellin variant is derived from 33MX further comprising a deletion (SEQ ID NO: 6, optionally without a terminal Histidine tag of SEQ ID NO: 5). In some embodiments, the flagellin variant comprises an amino acid sequence having at least 90%, at least 92%, at least 93%, at least 94%, at least 95%, at least 96%, at least 97%, or at least 98% sequence identity with SEQ ID NO: 6, optionally without a terminal Histidine tag (e.g., SEQ ID NO: 5). In some embodiments, the flagellin variant comprises at least one substitution or deletion mutation selected from amino acid residue position(s) corresponding to I18, F22, T23, S24, K27, I215, L216, Q217, T221, V223, A227, N228, Q229, V230, P231, Q232, N233, V234, L235, S236, and L237. In some embodiments, the flagellin variant comprises at least one substitution or deletion mutation selected from amino acid residue position(s) corresponding to I215, L216, Q217, T221, V223, A227, N228, Q229, V230, P231, Q232, N233, V234, L235, S236, and L237. In some embodiments, the substituted or deleted amino acid residue is any naturally-occurring amino acid. In further embodiments, the substituted or deleted amino acid residue is a hydrophilic or hydrophobic amino acid residue. In some embodiments, the hydrophilic amino acid residue is a polar and neutral of charge hydrophilic residue, selected from asparagine (N), glutamine (Q), serine (S), threonine (T), proline (P), and cysteine (C). In some embodiments, the hydrophilic amino acid residue is a polar and negatively charged hydrophilic residue, selected from aspartate (D) and glutamate (E). In further embodiments, the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y). In a further embodiment, the flagellin variant retains NF-kB signaling activity. In a further embodiment, the flagellin variant comprises T221D. In a further embodiment, the flagellin variant is TEM2-486D (SEQ ID NO: 29, optionally without a terminal Histidine tag).

In some embodiments, the flagellin variant comprises a truncation in one or more epitopes. In a further embodiment, the flagellin variant comprises a deletion in a N-terminal domain. In a further embodiment, the flagellin variant comprises a deletion in a C-terminal domain. In yet another embodiment, the flagellin variant comprises a deletion in epitope 1, epitope 2, or epitope 3. In yet a further embodiment, the flagellin variant comprises an amino acid sequence having at least 90% identity with SEQ ID NO: 1 and further comprises a deletion of amino acids 227-237. In further embodiments, the flagellin variant comprises an amino acid sequence having at least 90% identity with SEQ ID NO: 1, optionally wherein SEQ ID NO: 1 does not comprise a terminal Histidine tag (e.g., SEQ ID NO: 5). In yet a further embodiment, the flagellin variant comprises an amino acid sequence having at least 90% identity with SEQ ID NO: 6, optionally wherein SEQ ID NO: 6 does not comprise a terminal Histidine tag (e.g., SEQ ID NO: 5).

In some embodiments, the flagellin variants of the present invention have improved functional and pharmacological properties which, for example, allow for improved medical effects. In some embodiments, the flagellin variants of the present invention have similar or improved immunogenicity reduction and/or inflammasome activation reduction relative to entolimod/CBLB502 or other flagellin variants. In some embodiments, the flagellin variants of the present invention have similar or improved NF-kB activation and radioprotection and/or radiomitigation relative to entolimod/CBLB502 or other flagellin variants. In some embodiments, the flagellin variants have similar or improved pharmacokinetics leading to a proportionally stronger pharmacodynamic response (as detected by, for example, cytokine assays) relative to entolimod/CBLB502 or other flagellin variants. In some embodiments, the flagellin variant of the present invention demonstrates the same as or a similar or an improved pharmacokinetics profile compared with entolimod. In yet a further embodiment, the flagellin variant demonstrates increased or similar retention in the host as compared to retention of entolimod. In embodiments, administration of a flagellin variant of the present invention to a subject results in a substantially increased duration of bioavailability of the flagellin variant.

In some embodiments, the flagellin variants of the present invention have improved pharmacological properties, including reduced antigenicity, reduced immunogenicity, and reduced inflammasome activation, which, for example, allows for use in wide variety of disease states and patient types. A reduced antigenicity, immunogenicity, and inflammasome activation expands the medical applications for which the flagellin variants of the present invention can be used including, for example, medical applications requiring recurrent administration. In some embodiments, the decreased antigenicity translates to improved resistance against the neutralizing action of preexisting human antibodies (e.g. anti-flagellin) as well as those induced in response to entolimod/CBLB502 injection. In further embodiments, the flagellin variants have longer retention times in vivo. A longer retention time may allow the composition to be effective with fewer doses or with doses spaced further apart.

In some embodiments, the flagellin variants and methods of the present invention reduce or eliminate a side effect of radiotherapy and/or radiation exposure, including acute side effects, long-term side effects), or cumulative side effects. In various embodiments, the present methods reduce or eliminate a local or systemic side effect of radiotherapy and/or radiation exposure. In various embodiments, the side effect of radiotherapy and/or radiation exposure is one or more of fatigue, nausea and vomiting, damage to the epithelial surfaces (e.g., without limitation, moist desquamation), mouth, throat and stomach sores, intestinal discomfort (e.g., without limitation, soreness, diarrhea, and nausea), swelling, infertility, fibrosis, epilation, dryness (e.g. without limitation, dry mouth (xerostomia) and dry eyes (xerophthalmia), and dryness of the armpit and vaginal mucosa), lymphedema, heart disease, cognitive decline, radiation enteropathy (e.g. without limitation, atrophy, fibrosis and vascular changes, which may produce malabsorption, diarrhea, steatorrhea and bleeding with bile acid diarrhea and vitamin B12 malabsorption commonly found due to ileal involvement.

In some embodiments, the flagellin variant comprises a tag. In yet a further embodiment, the tag is attached to the N-terminus of the flagellin variant. In yet another embodiment, the tag is attached to the C-terminus of the flagellin variant.

In some embodiments, the flagellin variant comprises or consists of any one of the protein flagellin variants listed in Table 1. In some embodiments, the flagellin variant comprises or consists of an amino acid sequence of any one of SEQ ID NOs: 2 and 7-40. In further embodiments, the invention provides for a flagellin variant comprising or consisting of an amino acid sequence having at least 50%, at least 60%, at least 70%, at least 80%, at least 85%, at least 90%, at least 95%, at least 97%, at least 98%, at least 99%, at least 100% sequence identity with any one of SEQ ID NOs: 2 and 7-40. In some embodiments, the flagellin variant comprises or consists of the polypeptide of SEQ ID NO: 2 (a.k.a. 491TEMX/SE-2/GP532). In some embodiments, the flagellin variants may be at least 30-99% identical to any one of sequences SEQ ID NOs: 2 and 7-40. In some embodiments, the flagellin variant comprises an amino acid sequence having about 50%, or about 60%, or about 70%, or about 80%, or about 85%, or about 90%, or about 95%, or about 97%, or about 98%, or about 99%, or about 100% sequence identity to any one of sequences SEQ ID NO: 1, SEQ ID NO: 3, SEQ ID NO: 4, and SEQ ID NO: 6. In further embodiments, the flagellin variant of the invention comprises an amino acid sequence having about 50%, or about 60%, or about 70%, or about 80%, or about 85%, or about 90%, or about 95%, or about 97%, or about 98%, or about 99%, or about 100% sequence identity to any one of sequences SEQ ID NOs: 2, 7, 8, 9, 10, 11, 12, 13, 14, 15, 16, 23, 24, 25, 28, and 29.

### Uses of Flagellin Variants

In some embodiments, the flagellin variants stimulate Toll-like receptor activity (e.g. TLR5). The TLR family is composed of at least 10 members and is essential for innate immune defense against pathogens. The innate immune system recognizes conserved pathogen-associated molecular patterns (PAMPs). TLR may recognize a conserved structure that is particular to bacterial flagellin which may be composed of a large group of residues that are somewhat permissive to variation in amino acid content. Smith et al., Nat. Immunol. 4:1247-53 (2003) have identified 13 conserved amino acids in flagellin that are part of the conserved structure recognized by TLR5.

In some embodiments, the flagellin variant activates TLR5 signaling. In some embodiments, the flagellin variant activates TLR5 at the same levels, or levels similar to, entolimod/CBLB502 and/or other flagellin variants. Activation of TLR5 induces NF-κB-dependent promoters, which in turn activate numerous inflammatory-related cytokines. In further embodiments, the flagellin variants induce expression of proinflammatory cytokines. In further embodiments, the flagellin variants induce expression of anti-inflammatory molecules. In another embodiment, the flagellin variants induce expression of anti-apoptotic molecules. In yet a further embodiment, the flagellin variants induce expression of anti-bacterial molecules. The targets of NF-κB, include, but are not limited to, IL-1β, TNF-α, IL-6, IL-8, IL-18, G-CSF, TNFSF13B, keratinocyte chemoattractant (KC), BLIMP1/PRDM1, CCL5, CCL15, CCL17, CCL19, CCL20, CCL22, CCL23, CXCL1, CCL28, CXCL11, CXCL10, CXCL3, CXCL1, GRO-beta, GRO-gamma, CXCL1, ICOS, IFNG, IL-1A, IL-1B, URN, IL-2, IL-9, IL-10, IL-11, IL-12, IL-12B, IL-12A, IL-13, IL-15, IL-17, IL-23A, IL-27, EBI3, IFNB1, CXCL5, KC, liGp1, CXCL5, CXCL6, LTA, LTB, CCL2, CXCL9, MCP-1/JE, CCL3, CCL4, CXCL3, CCL20, CXCL10, CXCL5, CCL5, CCL1, TNFbeta, TNFSF10, TFF3, TNFSF15, CD86, complement component 8a, CCL27, defensin-β3, MIG, MIP-2, and/or NOD2/CARD15.

In some embodiments, activating TLR5 signaling may regulate CD4⁺ T-cell immune function by increasing the generation of regulatory T-cells (T_{regs}), decreasing LPS-induced ERK1/2 activation, and/or activating Natural Killer (NK) T-cells.

### Diseases and Methods of Treatment/Prevention

In various embodiments, the flagellin variants (and/or additional agents) and methods described herein are applicable to variety of disease states. In one aspect, the invention provides a method of stimulating TLR5 signaling comprising administering a flagellin variant of the invention to a subject in need thereof. Activating TLR5 signaling may have broad therapeutic applications, including, but not limited to treating cancer, protecting from radiation-induced or reperfusion-induced damage, acting as adjuvant in vaccines, or protecting cells from cytotoxic compounds.

In some embodiments, the flagellin variants of the invention, or fragments thereof may be provided as adjuvants to viral vaccines. In one embodiment, the flagellin variants or fragments thereof may be administered in conjunction with an influenza vaccine or antigen to elicit a greater host immune response to the influenza antigens. In yet a further embodiment, the flagellin variants of the invention, or fragments thereof may be provided as adjuvants to vaccines against parasites. In one embodiment, the flagellin variants or fragments thereof may be administered in conjunction with a Plasmodium vaccine or antigen to elicit a greater host immune response to the Plasmodium antigen.

In some embodiments, the flagellin variants of the present invention may be administered to protect cells from toxic conditions. In some embodiments, the flagellin variants may prevent liver cells from Fas-mediated injury. The flagellin variants of the invention may cause a decrease in liver enzymes in the peripheral blood and caspase activation.

Flagellin and previously described variants suffer from inflammasome activation. Without wishing to be bound by any one theory, it is thought that extracellular flagellin is able to activate the cytoplasmic NLRC4 inflammasome due to internalization of flagellin-TLR5 complexes. The NLRC4 inflammasome is one of a number of cytoplasmic multi-molecule complexes that is assembled following activation of its pattern recognition receptor (PRR) component by a microbial entity. In the case of NLRC4, the cytoplasmic Nod-like receptor (NLR) is activated by bacterial flagellin, which is also an agonist of Toll-like receptor 5 (TLR5) on the cell membrane. It is assumed that extracellular flagellin is able to activate the cytoplasmic NLRC4 inflammasome due to internalization of flagellin-TLR5 complexes. Once assembled, the inflammasome initiates a pro-inflammatory cascade involving caspase-1 activation and, subsequently, processing of pro-IL-1β to mature IL-1β, which is major pro-inflammatory cytokine. As a result of such inflammasome activation, subjects may experience undesirable side effects making therapeutic applications more difficult.

Cancers

In various embodiments, the present invention pertains to cancers and/or tumors; for example, the treatment or prevention of cancers and/or tumors. As used herein, “cancer” or “tumor” refers to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g. virus infected cells). A subject that has a cancer or a tumor is a subject having objectively measurable cancer cells present in the subject's body. Cancers which migrate from their original location and seed vital organs can eventually lead to the death of the subject through the functional deterioration of the affected organs. Hematopoietic cancers, such as leukemia, are able to out-compete the normal hematopoietic compartments in a subject, thereby leading to hematopoietic failure (in the form of anemia, thrombocytopenia and neutropenia) ultimately causing death.

The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis.

The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from, for example, melanoma, colon, breast, or prostate, and thus may be made up of cells that were originally skin, colon, breast, or prostate, respectively. The cancer may also be a hematological malignancy, which may be lymphoma. The cancer may invade a tissue such as liver, lung, bladder, or intestinal. The invaded tissue may express a TLR, while the cancer may or may not express a TLR.

Also provided herein is a method of reducing cancer recurrence, comprising administering to a mammal in need thereof a flagellin variant of the invention. The cancer may be or may have been present in a tissue that either does or does not express TLR, such as TLR5. The method may also prevent cancer recurrence. The cancer may be an oncological disease. The cancer may be a dormant tumor, which may result from the metastasis of a cancer. The dormant tumor may also be left over from surgical removal of a tumor. The cancer recurrence may be tumor regrowth, a lung metastasis, or a liver metastasis.

Representative cancers and/or tumors of the present invention may or may not express TLR5, and may include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The flagellin variants (and/or additional agents) and methods described herein are applicable metastatic diseases, including cancers and/or tumors. “Metastasis” refers to the spread of cancer from a primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases may be detected through the sole or combined use of magnetic resonance imaging (MRI) scans, computed tomography (CT) scans, blood and platelet counts, liver function studies, chest X-rays and bone scans in addition to the monitoring of specific symptoms.

In some embodiments, the invention relates to a method of treating a mammal suffering from a constitutively active NF-κB cancer comprising administering to the mammal a composition comprising a therapeutically effective amount of an agent that induces NF-κB activity, including the flagellin variants (and/or additional agents) described herein. The agent that induces NF-κB activity may be administered in combination with a cancer treatment.

In some embodiments, the present invention includes methods for treatment of side effects from cancer treatment comprising administering the flagellin variant (and/or additional agents) described herein. In some embodiments, the side effects from cancer treatment include alopecia, myelosuppression, renal toxicity, weight loss, pain, nausea, vomiting, diarrhea, constipation, anemia, malnutrition, hair loss, numbness, changes in tastes, loss of appetite, thinned or brittle hair, mouth sores, memory loss, hemorrhage, cardiotoxicity, hepatotoxicity, ototoxicity, and post-chemotherapy cognitive impairment.

In some embodiments, the present invention relates to a method of treating a mammal suffering from damage to normal tissue attributable to treatment of cancer, including but not limited to a constitutively active NF-κB cancer, comprising administering to the mammal a composition comprising a therapeutically effective amount of the flagellin variant (and/or additional agents) described herein.

Aging and Stress

In some embodiments, the present invention includes methods for modulation of cell aging (e.g., suppression and/or deceleration of mammalian cellular aging) comprising administering the flagellin variant (and/or additional agents) described herein.

For example, in some embodiments, the methods provided herein are to prevent or treat age-related diseases such as Alzheimer's disease, type II diabetes, macular degeneration, chronic inflammation-based pathologies (e.g., arthritis), and/or to prevent development of cancer types known to be associated with aging (e.g., prostate cancer, melanoma, lung cancer, colon cancer, etc.), and/or with the purpose to restore function and morphology of aging tissues (e.g., skin or prostate), and/or with the purpose to improve morphology of tissue impaired by accumulated senescent cells (e.g., cosmetic treatment of pigmented skin lesions), and/or with the purpose to improve the outcome of cancer treatment by radiation or chemotherapy, and/or with the purpose to prevent recurrent and metastatic disease in cancer patients by elimination of dormant cancer cells. The disclosure is suitable for prophylaxis and/or therapy of human and non-human animal diseases and aging and age-related disorders.

In various examples, the disclosure relates to methods of treating an individual suspected of having or at risk for developing an age-related disease, including but not necessarily limited to Alzheimer's disease, Type II diabetes, macular degeneration, or a disease comprising chronic inflammation, including but not necessarily limited to arthritis.

In some embodiments, the methods described herein or for treatment of a patient identified as having or at risk of having a cardiovascular disease or disorder, inflammatory disease or disorder, pulmonary disease or disorder, neurological disease or disorder, metabolic disease or disorder, dermatological disease or disorder, age-related disease or disorder, a premature aging disease or disorder, and a sleep disorder. Premature aging diseases and disorders include, but are not limited to Hutchinson-Gilford progeria or Werner's Syndrome.

In various embodiments, the present invention relates to treating or preventing senescence, for example by reducing, halting, or delaying the senescence. Without intending to be bound by any particular theory, cellular aging (senescence) is considered to be caused by overstimulation and overactivation of signal transduction pathways such as the mTOR pathway, especially when the cell cycle is blocked, leading to cellular hyperactivation and hyperfunction. In turn, this causes secondary signal resistance and compensatory incompetence. Both cellular hyperfunction and signal-resistance cause organ damage (including in distant organs), manifested as aging (subclinical damage) and age-related diseases (clinical damage), eventually leading to organismal death. Non-limiting example of markers of cellular aging are considered to be cellular hypertrophy, permanent loss of proliferative potential, large-flat cell morphology and beta-Gal staining. In various embodiments, the present invention relates to modulating any of the markers of cellular aging.

The aging process is manifested by a gradual accumulation of deficiencies in all major physiological functions, reduction of regeneration capabilities, impaired wound healing and increased risk of age-related diseases such as cancer, diabetes type 2, arthritis, Alzheimer and Parkinson diseases, atherosclerosis and others. Cumulatively, all these events can be described as a gradual increase in frailty and measured by a so-called “frailty index”. Age-related increase in frailty can be expedited in people or animals that underwent cancer treatment by chemotherapy and radiation, which can be interpreted as accelerated aging. The progression of natural aging, as well as aging accelerated by cancer treatment, can be dramatically slowed down by activation of natural innate immunity mechanism of response to infection with bacteria that have flagella—an organelle for active moving that is built with the protein named flagellin; presence of such bacteria in the body is recognized by a cell surface receptor named Toll-like receptor 5 (TLR5). Binding of the flagellin variant of the present invention to TLR5 triggers a physiological response leading to systemic mobilization of immune system accompanied with production of multiple bioactive factors (cytokines, chemokines, etc.) that have long-term effect on the organism manifested as a slowdown of frailty acquisition and improved health and quality of life of the treated organisms. Treatment with the flagellin variant of the present invention (and/or additional agents) capable of activation of TLR5 can be projected as an approach to prevent and treat natural aging and premature accelerated aging caused by cancer treatment and other types of poisoning.

Aging is a gradual systemic pathological transformation of mammalian organism advancing with time. It is associated with accumulation of multiple deficiencies in functions of all organs and tissues and reduced regeneration capabilities leading to development of age-related chronic diseases including atherosclerosis, diabetes, pulmonary fibrosis, blindness, dementia, kidney dysfunction, osteoarthritis, and low grade chronic sterile inflammation as well as other age-related diseases and disorders contemplated herein. These conditions frequently coincide with a gradual development of geriatric syndromes including frailty, cognitive impairment and immobility. Aging is a natural and unavoidable process. Underlying causes of aging are still disputable; however, two features of aging are generally accepted as universal: an increase in DNA damage and development of systemic sterile chronic inflammation, both considered as major contributors of age-related pathologies.

Exposure of younger individuals to genotoxic medical treatments or environment has been linked to a high risk of premature development of multiple aging-associated conditions listed above and considered as accelerated aging.

One of the most common medical treatments of this type is cancer treatment. Cancer treatment frequently involves exposure of humans and animals to genotoxic stresses leaving numerous normal cells with damaged DNA, provoking accumulation of senescent cells and acquisition of chronic systemic inflammation. These conditions increase the risk of multiple diseases commonly associated with natural aging such as abnormal thyroid function, decreased bone mineral density and increased osteoporosis, infertility, compromised tissue regeneration, cardiotoxicity, pulmonary fibrosis and chronic sterile inflammation. Acceleration of aging in cancer survivors is especially well documented in individuals that were successfully treated for cancer in their childhood. In fact, adults treated for childhood cancer are at increased risk of early development of chronic health conditions such as cardiovascular, pulmonary, hepatic, renal, and gonadal dysfunction, and secondary malignant neoplasms and increased rate of mortality. The rates of chronic diseases among survivors in their 20s are similar to rates among siblings in their 50s. Elevated rates of other aging-associated conditions, such as cognitive dysfunction, and reduced muscle strength, are also reported among childhood cancer survivors and appear decades earlier than expected. This and other studies suggest that some survivors of childhood cancer have a physiological frailty phenotype consistent with that found among older adults. Physiologic frailty among hematopoietic cell transplantation (HCT) survivors also suggests accelerated aging and is a predictor for premature mortality. Rates of frailty were eightfold higher among HCT survivors than among their siblings. Among survivors of HCT at least 10 years after transplant, the 15-year cumulative incidence of severe/life-threatening/fatal conditions was 41%.

The term “age-related disease” includes but is not limited to a disease in an adult such as cancer, a metabolic disease, cardiovascular disease, tobacco-related disease, or skin wrinkles. Cancer includes but is not limited to prostate cancer, colon cancer, lung cancer, squamous cell cancer of the head and neck, esophageal cancer, hepatocellular carcinoma, gastric cancer, pancreatic cancer, ovarian cancer, or breast cancer. Age-related or tobacco-related disease includes cardiovascular disease, cerebrovascular disease, peripheral vascular disease, Alzheimer's disease, osteoarthritis, cardiac diastolic dysfunction, benign prostatic hypertrophy, aortic aneurysm, or emphysema.

There are several comprehensive approaches for quantitative assessment of aging-related accumulation of deficits and frailty in humans and animals. Individual organisms are heterogeneous in their health status and the rate of aging. To account for such heterogeneity, the Frailty Index (FI) has been introduced as a numerical score which is a ratio of the deficits present in a person to the total number of deficits considered in the study. Changes in the FI characterize the rate of individual aging. A similar approach has been applied to laboratory animals. Frailty index is considered as a reliable and broadly accepted measure of “biological’ age” and the degree of general health decline indicative of a reduction in the quality of life.

There are currently no drugs or treatments that are conventionally used in medicine for prophylaxis and treatment of aging. Extension of healthy life and longevity has been documented by caloric restriction. A similar effect can be reached using mTOR inhibitors such as rapamycin. Both require long-term applications. In some embodiments, the present invention provides for a method of modulating cell aging comprising administering a flagellin variant described herein in conjunction with a mTOR inhibitor, including, but not limited to, various rapalogs (e.g., rapamycin and its analogs). Pharmacological agents capable of slowing down the process of advancement of age-related frailty—both naturally occurring and accelerated by cancer treatment—are desperately needed. If developed, they would have an unlimited market as agents applicable to the treatment of a pathology that hits 100% of the population.

In some embodiments, the present invention includes methods for treatment of stress comprising administering the flagellin variant (and/or additional agents) described herein. This invention also relates to a method of treating a subject suffering from damage to normal tissue attributable to stress, comprising administering to the mammal a composition comprising a therapeutically effective amount of a flagellin variant (and/or additional agents). The stress may be attributable to any source including, but not limited to, radiation, wounding, poisoning, infection, and temperature shock.

In some embodiments, the flagellin variant (and/or additional agents) may be administered at any point prior to exposure to the stress including, but not limited to, about 48 hr, about 46 hr, about 44 hr, about 42 hr, about 40 hr, about 38 hr, about 36 hr, about 34 hr, about 32 hr, about 30 hr, about 28 hr, about 26 hr, about 24 hr, about 22 hr, about 20 hr, about 18 hr, about 16 hr, about 14 hr, about 12 hr, about 10 hr, about 8 hr, about 6 hr, about 4 hr, about 3 hr, about 2 hr, or about 1 hr prior to exposure. In some embodiments, the flagellin variant may be administered at any point after exposure to the stress including, but not limited to, about 1 hr, about 2 hr, about 3 hr, about 4 hr, about 6 hr, about 8 hr, about 10 hr, about 12 hr, about 14 hr, about 16 hr, about 18 hr, about 20 hr, about 22 hr, about 24 hr, about 26 hr, about 28 hr, about 30 hr, about 32 hr, about 34 hr, about 36 hr, about 38 hr, about 40 hr, about 42 hr, about 44 hr, about 46 hr, or about 48 hr after exposure.

Mitigation and Prevention of Radiation Damage

In still other embodiments, the present invention relates to treatment of radiation related diseases or damage. In specific embodiments, the present invention relates to mitigation of or prevention and/or protection from radiation related diseases.

In one embodiment, the present invention relates to the protection of cells from the effects of exposure to radiation. In some embodiments, the present invention pertains to a method of protecting a subject from radiation comprising administering a flagellin variant (and/or additional agents) described herein. In some embodiments, the radiation is ionizing radiation. In some embodiments, the ionizing radiation is sufficient to cause gastrointestinal syndrome or hematopoietic syndrome. In some embodiments, the flagellin variant (and/or additional agents) described herein is administered in combination with a radioprotectant e.g. an antioxidant (e.g. amifostine and vitamin E), a cytokine (e.g. a stem cell factor), etc. In some embodiments, the flagellin variant (and/or additional agents) described herein is administered prior to, together with, or after radiation. In some embodiments, the flagellin variant (and/or additional agents) described herein is administered in combination with a growth factor (e.g. keratinocyte growth factor), a steroid (e.g. 5-androstenediol), ammonium trichloro(dioxoethylene-O,O′)tellurate, thyroid protecting agents (e.g. Potassium iodide (KI)), anti-nausea agents, anti-diarrhea agents, analgesics, anxiolytics, sedatives, cytokine therapy, antibiotics, antifungal agents, and/or antiviral agents.

In some embodiments, the present invention pertains to a method of treating and/or mitigating apoptosis-mediated tissue damage in a subject, comprising administering to a subject in need thereof a composition comprising a flagellin variant (and/or additional agents) described herein. In some embodiments the apoptosis is attributable to cellular stress. In some embodiments, the flagellin variant (and/or additional agents) described herein is administered prior to, together with, or after the tissue damage. In some embodiments, the cellular stress is radiation. In some embodiments, the flagellin variant (and/or additional agents) is administered in combination with a radioprotectant (e.g. an antioxidant (e.g. amifostine and vitamin E), a cytokine (e.g. a stem cell factor), etc.

Injury and death of normal cells from ionizing radiation is a combination of a direct radiation-induced damage to the exposed cells and an active genetically programmed cell reaction to radiation-induced stress resulting in a suicidal death or apoptosis. Apoptosis plays a key role in massive cell loss occurring in several radiosensitive organs (e.g., hematopoietic and immune systems, epithelium of digestive tract, etc.), the failure of which determines general radiosensitivity of the organism. In some embodiments, administration of the flagellin variants of the invention to a subject in need thereof suppresses apoptosis in cells. In some embodiments, the flagellin variants of the invention are administered to a subject undergoing cancer radiotherapy treatment to protect healthy cells from the damaging effects of the radiation treatment.

Exposure to ionizing radiation (IR) may be short- or long-term, and/or it may be applied as a single or multiple doses and/or it may be applied to the whole body or locally. The present invention, in some embodiments, pertains to nuclear accidents or military attacks, which may involve exposure to a single high dose of whole body irradiation (sometimes followed by a long-term poisoning with radioactive isotopes). The same is true (with strict control of the applied dose), for example, for pretreatment of patients for bone marrow transplantation when it is necessary to prepare hematopoietic organs for donor's bone marrow by “cleaning” them from the host blood precursors. Cancer treatment may involve multiple doses of local irradiation that greatly exceeds lethal dose if it were applied as a total body irradiation. Poisoning or treatment with radioactive isotopes results in a long-term local exposure to radiation of targeted organs (e.g., thyroid gland in the case of inhalation of ¹²⁵I). Further, there are many physical forms of ionizing radiation differing significantly in the severity of biological effects.

At the molecular and cellular level, radiation particles are able to produce breakage and cross-linking in the DNA, proteins, cell membranes and other macromolecular structures. Ionizing radiation also induces the secondary damage to the cellular components by giving rise to the free radicals and reactive oxygen species (ROS). Multiple repair systems counteract this damage, such as, several DNA repair pathways that restore the integrity and fidelity of the DNA, and antioxidant chemicals and enzymes that scavenge the free radicals and ROS and reduce the oxidized proteins and lipids. Cellular checkpoint systems detect the DNA defects and delay cell cycle progression until damage is repaired or decision to commit cell to growth arrest or programmed cell death (apoptosis) is reached.

Radiation can cause damage to mammalian organism ranging from mild mutagenic and carcinogenic effects of low doses to almost instant killing by high doses. Overall radiosensitivity of the organism is determined by pathological alterations developed in several sensitive tissues that include hematopoietic system, reproductive system and different epithelia with high rate of cell turnover.

Acute pathological outcome of gamma irradiation leading to death is different for different doses and may be determined by the failure of certain organs that define the threshold of organism's sensitivity to each particular dose. Thus, lethality at lower doses occurs, for example, from bone marrow aplasia, while moderate doses kill faster, for example, by inducing a gastrointestinal (GI) syndrome. Very high doses of radiation can cause almost instant death eliciting neuronal degeneration.

Organisms that survive a period of acute toxicity of radiation can suffer from long-term remote consequences that include radiation-induced carcinogenesis and fibrosis developing in exposed organs (e.g., kidney, liver or lungs) in the months and years after irradiation.

Cellular DNA is a major target of IR that causes a variety of types of DNA damage (genotoxic stress) by direct and indirect (e.g. free radical-based) mechanisms. All organisms maintain DNA repair system capable of effective recovery of radiation-damaged DNA; errors in DNA repair process may lead to mutations.

In some embodiments, the radiation exposure experienced by the subject is a consequence of cancer radiotherapy treatment. Tumors are generally more sensitive to gamma radiation and can be treated with multiple local doses that cause relatively low damage to normal tissue. Nevertheless, in some instances, damage of normal tissues is a limiting factor in application of gamma radiation for cancer treatment. The use of gamma-irradiation during cancer therapy by conventional, three-dimensional conformal or even more focused BeamCath delivery has also dose-limiting toxicities caused by cumulative effect of irradiation and inducing the damage of the stem cells of rapidly renewing normal tissues, such as bone marrow and gastrointestinal (GI) tract. Administration of the flagellin variants of the invention may protect the patient's healthy cells from radiation damage without affecting the radiosensitivity of the tumor cells.

In some embodiments, the subject has been exposed to lethal doses of radiation. At high doses, radiation-induced lethality is associated with so-called hematopoietic and gastrointestinal radiation syndromes. Hematopoietic syndrome is characterized by loss of hematopoietic cells and their progenitors making it impossible to regenerate blood and lymphoid system. Death usually occurs as a consequence of infection (result of immunosuppression), hemorrhage and/or anemia. GI syndrome is caused by massive cell death in the intestinal epithelium, predominantly in the small intestine, followed by disintegration of intestinal wall and death from bacteriemia and sepsis. Hematopoietic syndrome usually prevails at the lower doses of radiation and leads to the more delayed death than GI syndrome.

In the past, radioprotectants were typically antioxidants-both synthetic and natural. More recently, cytokines and growth factors have been added to the list of radioprotectants; the mechanism of their radioprotection is considered to be a result of facilitating the effects on regeneration of sensitive tissues. There is no clear functional distinction between both groups of radioprotectants, however, since some cytokines induce the expression of the cellular antioxidant proteins, such as manganese superoxide dismutase (MnSOD) and metallothionein.

The measure of protection for a particular agent may be expressed by dose modification factor (DMF or DRF). DMF is determined by irradiating the radioprotector treated subject and untreated control subjects with a range of radiation doses and then comparing the survival or some other endpoints. DMF is commonly calculated for 30-day survival (LD50/30 drug-treated divided by LD50/30 vehicle-treated) and quantifies the protection of the hematopoietic system. In order to estimate gastrointestinal system protection, LD50 and DMF are calculated for 6- or 7-day survival.

The flagellin variants described herein possess strong pro-survival activity at the cellular level and on the organism as a whole. In response to super-lethal doses of radiation, the flagellin variants described herein may inhibit both gastrointestinal and hematopoietic syndromes, which are major causes of death from acute radiation exposure. As a result of these properties, the flagellin variants described herein may be used to treat the effects of natural radiation events and nuclear accidents. Moreover, the flagellin variants described herein can be used in combination with other radioprotectants, thereby, dramatically increasing the scale of protection from ionizing radiation.

As opposed to conventional radioprotective agents (e.g., scavengers of free radicals), anti-apoptotic agents may not reduce primary radiation-mediated damage but may act against secondary events involving active cell reaction on primary damage, therefore complementing the existing lines of defense. Pifithrin-alpha, a pharmacological inhibitor of p53 (a key mediator of radiation response in mammalian cells), is an example of this new class of radioprotectants. However, the activity of p53 inhibitors is limited to protection of the hematopoietic system and has no protective effect in digestive tract (gastrointestinal syndrome), therefore reducing therapeutic value of these compounds.

The flagellin variants described herein may be used as a radioprotective agent to extend the range of tolerable radiation doses by increasing radioresistance of humans beyond the levels achievable by currently available measures (shielding and application of existing bioprotective agents) and drastically increase the chances of crew survival in case of nuclear accidents or large-scale solar particle events, for example.

The flagellin variants described herein are also useful for treating irreplaceable cell loss caused by low-dose irradiation, for example, in the central nervous system and reproductive organs. The flagellin variants described herein may also be used during cancer chemotherapy to treat the side effects associated with chemotherapy, including alopecia, myelosuppression, renal toxicity, weight loss; pain, nausea, vomiting, diarrhea, constipation, anemia, malnutrition, hair loss, numbness, changes in tastes, loss of appetite, thinned or brittle hair, mouth sores, memory loss, hemorrhage, cardiotoxicity, hepatotoxicity, ototoxicity, and post-chemotherapy cognitive impairment.

In one embodiment, a mammal is treated for exposure to radiation, comprising administering to the mammal a composition comprising a therapeutically effective amount of a flagellin variant. The flagellin variant may be administered in combination with one or more radioprotectants. The one or more radioprotectants may be any agent that treats the effects of radiation exposure including, but not limited to, antioxidants, free radical scavengers and cytokines.

The flagellin variants described herein may inhibit radiation-induced programmed cell death in response to damage in DNA and other cellular structures. In some embodiments, the flagellin variants described herein may not deal with damage at the cellular and may not prevent mutations. Free radicals and reactive oxygen species (ROS) are the major cause of mutations and other intracellular damage. Antioxidants and free radical scavengers are effective at preventing damage by free radicals. The combination of a flagellin variant and an antioxidant or free radical scavenger may result in less extensive injury, higher survival, and improved health for mammals exposed to radiation. Antioxidants and free radical scavengers that may be used in the practice of the invention include, but are not limited to, thiols, such as cysteine, cysteamine, glutathione and bilirubin; amifostine (WR-2721); vitamin A; vitamin C; vitamin E; and flavonoids such as Indian holy basil (Ocimum sanctum), orientin and vicenin.

The flagellin variants described herein may also be administered in combination with a number of cytokines and growth factors that confer radioprotection by replenishing and/or protecting the radiosensitive stem cell populations. Radioprotection with minimal side effects may be achieved by the use of stem cell factor (SCF, c-kit ligand), Flt-3 ligand, and interleukin-1 beta. Protection may be achieved through induction of proliferation of stem cells (all mentioned cytokines), and prevention of their apoptosis (SCF). The treatment allows accumulation of leukocytes and their precursors prior to irradiation thus enabling quicker reconstitution of the immune system after irradiation. SCF efficiently rescues lethally irradiated mice with DMF in range 1.3-1.35 and is also effective against gastrointestinal syndrome. Flt-3 ligand also provides strong protection in mice and rabbits.

Several factors, while not cytokines by nature, stimulate the proliferation of the immunocytes and may be used in combination with the flagellin variants described herein. For example, 5-AED (5-androstenediol) is a steroid that stimulates the expression of cytokines and increases resistance to bacterial and viral infections. Synthetic compounds, such as ammonium tri-chloro(dioxoethylene-O,O′—) tellurate (AS-101), may also be used to induce secretion of numerous cytokines and for combination with the flagellin variants described herein.

Growth factors and cytokines may also be used to provide protection against the gastrointestinal syndrome. Keratinocyte growth factor (KGF) promotes proliferation and differentiation in the intestinal mucosa, and increases the post-irradiation cell survival in the intestinal crypts. Hematopoietic cytokine and radioprotectant SCF may also increase intestinal stem cell survival and associated short-term organism survival.

The flagellin variants described herein may offer protection against both gastrointestinal (GI) and hematopoietic syndromes. Such compositions may be used in combination with one or more inhibitors of GI syndrome (including, but are not limited to, cytokines such as SCF and KGF).

The flagellin variant may be administered at any point prior to exposure to radiation including, but not limited to, about 48 hr, about 46 hr, about 44 hr, about 42 hr, about 40 hr, about 38 hr, about 36 hr, about 34 hr, about 32 hr, about 30 hr, about 28 hr, about 26 hr, about 24 hr, about 22 hr, about 20 hr, about 18 hr, about 16 hr, about 14 hr, about 12 hr, about 10 hr, about 8 hr, about 6 hr, about 4 hr, about 3 hr, about 2 hr, or about 1 hr prior to exposure. The flagellin variant may be administered at any point after exposure to radiation including, but not limited to, about 1 hr, about 2 hr, about 3 hr, about 4 hr, about 6 hr, about 8 hr, about 10 hr, about 12 hr, about 14 hr, about 16 hr, about 18 hr, about 20 hr, about 22 hr, about 24 hr, about 26 hr, about 28 hr, about 30 hr, about 32 hr, about 34 hr, about 36 hr, about 38 hr, about 40 hr, about 42 hr, about 44 hr, about 46 hr, or about 48 hr after exposure to radiation.

In various embodiments, the present methods and compositions provide treatment or prevention of radiation-related disorders, such as ARS. In various embodiments, the treatments described herein reduce morbidity or mortality of an exposed population of human patients or accelerates recovery from symptoms of ARS. ARS often presents as a sequence of phased symptoms, which may vary with individual radiation sensitivity, type of radiation, and the radiation dose absorbed. Generally, without wishing to be bound by theory, the extent of symptoms will heighten and the duration of each phase will shorten with increasing radiation dose. ARS can be divided into three phases: prodromal phase (a.k.a. N-V-D stage), latent period and manifest illness. In various embodiments, the flagellin variants (and/or additional agents), as described herein, may be administered to a human patient in any one of these three stages (i.e. the flagellin variants (and/or additional agents) may be administered to a human patient in the prodromal phase, the flagellin variants (and/or additional agents) may be administered to a human patient in latent period, or the flagellin variants (and/or additional agents) may be administered to a human patient in manifest illness stage).

In the prodromal phase there is often a relatively rapid onset of nausea, vomiting, and malaise. Use of antiemetics, (e.g. oral prophylactic antiemetics) such as granisetron (KYTRIL), ondansetron (ZOFRAN), and 5-HT3 blockers with or without dexamethasone, may be indicated in situations where high-dose radiological exposure has occurred, is likely, or is unavoidable. Accordingly, in various embodiments, the flagellin variants (and/or additional agents) may be administered to a human patient in receiving an anti-emetic agent or CBLB502 may be administered to a human patient in combination with an anti-emetic agent. For example, the flagellin variants (and/or additional agents) may also be added to the following antiemetic regimens: Ondansetron: initially 0.15 mg/kg IV; a continuous IV dose option consists of 8 mg followed by 1 mg/h for the next 24 hours. Oral dose is 8 mg every 8 hours as needed or Granisetron (oral dosage form): dose is usually 1 mg initially, then repeated 12 hours after the first dose. Alternatively, 2 mg may be taken as one dose. IV dose is based on body weight; typically 10 μg/kg (4.5 μg/lb) of body weight.

In the latent period, a human patient may be relatively symptom free. The length of this phase varies with the dose. The latent phase is longest preceding the bone-marrow depression of the hematopoietic syndrome and may vary between about 2 and 6 weeks. The latent period is somewhat shorter prior to the gastrointestinal syndrome, lasting from a few days to a week. It is shortest of all preceding the neurovascular syndrome, lasting only a matter of hours. These times are variable and may be modified by the presence of other disease or injury. Manifest illness presents with the clinical symptoms associated with the major organ system injured (marrow, intestinal, neurovascular).

In some embodiments, the present invention relates to the mitigation of, or protection of cells from, the effects of exposure to radiation. In some embodiments, the present invention pertains to a method of mitigating and/or protecting a human patient from radiation comprising administering the flagellin variants (and/or additional agents). In some embodiments, the radiation is ionizing radiation. In some embodiments, the ionizing radiation is sufficient to cause gastrointestinal syndrome or hematopoietic syndrome.

In some embodiments, the ARS comprises one of more of gastrointestinal syndrome; hematopoietic syndrome; neurovascular syndrome; apoptosis-mediated tissue damage, wherein the apoptosis is optionally attributable to cellular stress; and ionizing radiation induced apoptosis tissue damage.

Hematopoietic syndrome (a.k.a. bone marrow syndrome) is characterized by loss of hematopoietic cells and their progenitors making it impossible to regenerate blood and lymphoid system. This syndrome is often marked by a drop in the number of blood cells, i.e., aplastic anemia. This may result in infections (e.g. opportunistic infections) due to a low amount of white blood cells, bleeding due to a lack of platelets, and anemia due to few red blood cells in the circulation. These changes can be detected by blood tests after receiving a whole-body acute dose. Conventional trauma and burns resulting from a bomb blast are complicated by the poor wound healing caused by hematopoietic syndrome, increasing mortality. Death may occur as a consequence of infection (result of immunosuppression), hemorrhage and/or anemia. Hematopoietic syndrome usually prevails at the lower doses of radiation and leads to the more delayed death than GI syndrome.

Gastrointestinal syndrome is caused by massive cell death in the intestinal epithelium, predominantly in the small intestine, followed by disintegration of intestinal wall and death from bacteriemia and sepsis. Symptoms of this form of radiation injury include nausea, vomiting, loss of appetite, loss of absorptive capacity, hemorrhage in denuded areas, and abdominal pain. Illustrative systemic effects of gastrointestinal syndrome include malnutrition, dehydration, renal failure, anemia, sepsis, etc. Without treatment (including, for example, bone marrow transplant), death is common (e.g. via infection from intestinal bacteria). In some embodiments, the flagellin variants (and/or additional agents), may be used in combination with bone marrow transplant. In some embodiments, the flagellin variants (and/or additional agents), may be used in combination with one or more inhibitors of GI syndrome and/or any of the additional agents described herein.

Neurovascular syndrome presents with neurological symptoms such as dizziness, headache, or decreased level of consciousness, occurring within minutes to a few hours, and with an absence of vomiting. Additional symptoms include extreme nervousness and confusion; severe nausea, vomiting, and watery diarrhea; loss of consciousness; and burning sensations of the skin. Neurovascular syndrome is commonly fatal.

In some embodiments, the present invention provides a method for reducing the risk of death following exposure to irradiation comprising administering an effective amount of the flagellin variants (and/or additional agents) In some embodiments, the radiation is potentially lethal, and, optionally, occurs as the result of a radiation disaster. In various embodiments, the flagellin variant (and/or an additional agent) is administered within about 25 hours following radiation exposure. In some embodiments, the present invention provides a method for reducing the risk of death following exposure to potentially lethal irradiation occurring as the result of a radiation disaster, comprising administering the flagellin variants (and/or additional agents) within about 25 hours following radiation exposure.

In various embodiments, the flagellin variants (and/or additional agents) are administered to a patient who has been exposed to a high dose of radiation, namely a whole body dose. In various embodiments, the high dose of radiation may not be uniform. In various embodiments, the ARS is a result of a high dose of radiation. In various embodiments, the high dose of radiation is about 2.0 Gy, or about 2.5 Gy, or about 3.0 Gy, or about 3.5 Gy, or about 4.0 Gy, or about 4.5 Gy, or about 5 Gy, or about 10 Gy, or about 15 Gy, or about 20 Gy, or about 25 Gy, or about 30 Gy. In various embodiments, the high dose of radiation is about 5 to about 30 Gy, or about 10 to 25 Gy, or about 15 to 20 Gy. In some embodiments, the high dose of radiation is assessed by one or more of physical dosimetry and/or biological dosimetry (e.g. multiparameter dose assessments), cytogenics (e.g. chromosomal analysis for, for example, blood samples (including, by way of non-limiting example, dicentric analysis). In various embodiments, whole-body radiation doses can be divided into sublethal (<2 Gy), potentially lethal (2-10 Gy), and supralethal (>10 Gy).

Reperfusion Injuries

In some embodiments, the present invention pertains to a method of treating the effects of reperfusion on a subject's tissue comprising administering the flagellin-related compositions (and/or additional agents) described herein. The flagellin-related compositions (and/or additional agents) described herein may be administered in combination with an antioxidant, such as, for example, amifostine and vitamin E.

Reperfusion may be caused by an injury, which may be ischemia or hypoxia. The ischemia may result from a condition such as, for example, tachycardia, infarction, hypotension, embolism, thromboemoblism (blood clot), sickle cell disease, localized pressure to extremities to the body, and tumors. The hypoxia may be selected from hypoxemic hypoxia (carbon monoxide poisoning; sleep apnea, chronic obstructive pulmonary disease, respiratory arrest; shunts), anemic hypoxia (O₂ content low), hypoxemic hypoxia, and histotoxic hypoxia. The localized pressure may be due to a tourniquet.

The flagellin-related compositions (and/or additional agents) described herein may be administered prior to, together with, or after the influx of oxygen. The tissue may be for example, the GI tract, lung, kidney, liver, cardiovascular system, blood vessel endothelium, central nervous system, peripheral nervous system, muscle, bone, and hair follicle.

Reperfusion may damage a body component when blood supply returns to the body component after the injury. The effects of reperfusion may be more damaging to the body component than the injury itself. There are several mechanism and mediators of reperfusion including, for example, oxygen free radicals, intracellular calcium overload, and endothelial dysfunction. Excessive quantities of reactive oxygen species, when reintroduced into a previously injured body component, undergo a sequential reduction leading to the formation of oxygen free radicals. Potent oxidant radicals, such as superoxide anion, hydroxyl radical, and peroxynitrite may be produced within the first few minutes of reflow to the body component and may play a crucial role in the development of reperfusion injury. Oxygen free radicals also can be generated from sources other than reduction of molecular oxygen. These sources include enzymes, such as, for example, xanthine oxidase, cytochrome oxidase, and cyclooxygenase, and the oxidation of catecholamines.

Reperfusion is also a potent stimulus for neutrophil activation and accumulation, which in turn serve as potent stimuli for reactive oxygen species production. Specifically, the main products of the neutrophil respiratory burst are strong oxidizing agents including hydrogen peroxide, free oxygen radicals and hypochlorite. Neutrophils are the most abundant type of phagocyte, normally representing 50 to 60% of the total circulating leukocytes, and are usually the first cells to arrive at the site of injured body component. Oxygen-derived free radicals produce damage by reacting with polyunsaturated fatty acids, resulting in the formation of lipid peroxides and hydroperoxides that damage the body component and impair the function of membrane-bound enzyme systems. Free radicals stimulate the endothelial release of platelet activating factor and chemokines such as neutrophil activator factor, chemokine (C—X—C motif) ligand 1, and chemokine (C—X—C motif) ligand 1 which attracts more neutrophils and amplifies the production of oxidant radicals and the degree of reperfusion injury. Reactive oxygen species also quench nitric oxide, exaggerating endothelial injury and tissue cell dysfunction. In addition to an increased production, there is also a relative deficiency in endogenous oxidant scavenging enzymes, which further exaggerates free radical-mediated cardiac dysfunction.

Reperfusion may further result in marked endothelial cell dysfunction. Endothelial dysfunction facilitates the expression of a prothrombotic phenotype characterized by platelet and neutrophil activation, important mediators of reperfusion. Once neutrophils make contact with the dysfunctional endothelium, they are activated, and in a series of well-defined steps (rolling, firm adherence, and transmigration) they migrate into areas of tissue injury through endothelial cell junctions as part of the innate immune response.

Changes in intracellular calcium homeostasis play an important role in the development of reperfusion. Reperfusion may be associated with an increase in intracellular calcium; this effect may be related to increased sarcolemmal calcium entry through L-type calcium channels or may be secondary to alterations in sarcoplasmic reticulum calcium cycling. In addition to intracellular calcium overload, alterations in myofilament sensitivity to calcium have been implicated in reperfusion. Activation of calcium-dependent proteases (calpain I) with resultant myofibril proteolysis has been suggested to underscore reperfusion injury, as has proteolysis of troponin.

Reperfusion of tissue cells subjected to an injury had an altered cellular metabolism, which in turn may contribute to delayed functional recovery. For example, an injury may induce anaerobic metabolism in the cell with a net production of lactate. Lactate release persists during reperfusion, suggesting a delayed recovery of normal aerobic metabolism. Likewise, the activity of mitochondrial pyruvate dehydrogenase (PDH) may be inhibited up to 40% after an injury and may remain depressed for up to 30 minutes after reperfusion.

Each of these events during reperfusion can lead to stress to the tissue cells and programmed cell death (apoptosis) and necrosis of the tissue cells. Apoptosis normally functions to “clean” tissues from wounded and genetically damaged cells, while cytokines serve to mobilize the defense system of the organism against the pathogen. However, under conditions of severe injury both stress response mechanisms can by themselves act as causes of death.

In various embodiments, the effects of reperfusion may be caused by an injury to the body. The injury may be due to ischemia, hypoxia, an infarction, or an embolism. Treatment of the injury may lead to reperfusion and further damage to the body component.

Ischemia may be an absolute or relative shortage of blood supply to a body component. Relative shortage may be a mismatch, however small, of blood supplied (oxygen delivery) to a body component versus blood required to a body component for the adequate oxygenation. Ischemia may also be an inadequate flow of blood to a part of the body due to a constriction or blockage of blood vessels supplying it and may affect any body component in the body. Insufficient blood supply causes body components to become hypoxic, or, if no oxygen is supplied at all, anoxic. This may cause necrosis. The mechanisms of ischemia may vary greatly. For example, ischemia to any body component may be due to tachycardia (abnormally rapid beating of the heart), atherosclerosis (lipid-laden plaque obstructing the lumen of arteries), hypotension (low blood pressure in septic shock, heart failure), thromboembolisms (blood clots), outside compression of blood vessels (tumor), embolisms (foreign bodies in the circulation, e.g., amniotic fluid embolism), sickle cell disease (abnormally shaped hemoglobin), infarctions, induced g-forces which restrict the blood flow and force the blood to extremities of the body, localized extreme cold due to frostbite, ice, improper cold compression therapy, and any other force that restricts blood flow to the extremities such as a tourniquet. Force to restrict blood flow to extremities may be required due to severe lacerations, incisions, puncture such as a knifing, crushing injuries due to blunt force trauma, and ballistic trauma due to gunshot or shrapnel wounds. Ischemia may be a feature of heart diseases, ischemic colitis, transient ischemia attacks, cerebrovascular accidents, acute renal injury, ruptured arteriovenous malformations, and peripheral artery occlusive disease.

Hypoxia may be a deprivation of adequate supply of oxygen. Hypoxia may be pathological condition in which the body as a whole (generalized hypoxia) or region of the body (tissue hypoxia) is deprived of adequate oxygen supply. A variation in levels of arterial oxygen may be due to a mismatch between supply and demand of oxygen by body components. A complete deprivation of oxygen supply is anoxia. Hypoxia may be hypoxemic hypoxia, anemic hypoxia, hypoxemic hypoxia, histotoxic hypoxia, histotoxic hypoxia, and ischemic hypoxia.

Hypoxemic hypoxia may be an inadequate supply of oxygen to the body as a whole caused by low partial pressure of oxygen in arterial blood. Hypoxemic hypoxia may be due to low partial pressure of atmospheric oxygen such as at high altitudes, replacement of oxygen in breathing mix of a modified atmosphere such as a sewer, replacement of oxygen intentionally as in recreational use of nitrous oxide, a decrease in oxygen saturation of the blood due to sleep apnea, or hypopnea, inadequate pulmonary ventilation such as chronic obstructive pulmonary disease or respiratory arrest, anatomical or mechanical shunts in the pulmonary circulation or a right to left shunt in the heart and lung. Shunts may cause collapsed alveoli that are still perfused or a block in ventilation to an area of the lung. Shunts may present blood meant for the pulmonary system to not be ventilated and prevent gas exchange because the blood vessels empty into the left ventricle and the bronchial circulation, which supplies the bronchi with oxygen.

Anemia hypoxia may be the total oxygen content is reduced but the arterial oxygen pressure is normal. Hypoxemic hypoxia may be when blood fails to deliver oxygen to target body components. Hypoxemic hypoxia may be caused by carbon monoxide poisoning which inhibits the ability of hemoglobin to release the oxygen bound to it, or methaemoglobinaemia, an abnormal hemoglobin that accumulates in the blood. Histotoxic hypoxia may be due to being unable to effectively use oxygen due to disabled oxidative phosphorylation enzymes.

Infarction is a type of pathological condition that can cause ischemia. Infarction may be a macroscopic area of necrotic tissue caused the loss of an adequate blood supply due to an occlusion. The infarction may be a white infarction composed of platelets and causes necrosis in organ tissues such as heart, spleen, and kidneys. The infarction may be a red infarction composed of red blood cells and fibrin strands in organ tissues of the lung. Disease associated with infarction may include myocardial infarction, pulmonary embolism, cerebrovascular accident (stroke), acute renal failure, peripheral artery occlusive disease (example being gangrene), antiphospholipid syndrome, sepsis, giant cell arthritis, hernia, and volvulus.

Embolism is a type of pathological condition that can cause ischemia. Embolism may be an object that migrates from one part of the body and causes an occlusion or blockage of a blood vessel in another part of the body. An embolism may be thromboembolism, fat embolism, air embolism, septic embolism, tissue embolism, foreign body embolism, amniotic fluid embolism. Thromboembolism may be a blood clot that is completely or partially detached from the site of thrombosis. Fat embolism may be endogenous fat tissues that escape into the blood circulation. The fracture of bones is one example of a leakage of fat tissue into the ruptured vessels and arteries. Air embolism may be a rupture of alveoli and inhaled air that leaks into the blood vessels. The puncture of the subclavian vein or intravenous therapy are examples of leakage of air into the blood vessels. A gas embolism may be gasses such as nitrogen and helium because insoluble and forming small bubbles in the blood.

### Pharmaceutically Acceptable Salts and Excipients

The flagellin variants (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

Further, any flagellin variants (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

### Formulations, Administration, Dosing, and Treatment Regimens

The present invention includes the described flagellin variants (and/or additional agents) in various formulations. Any flagellin variant (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the flagellin variants (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the flagellin variants (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, any flagellin variant (and/or additional agents) described herein is formulated in accordance with routine procedures as a composition adapted for a mode of administration described herein.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, the administering is effected orally or by parenteral injection. The mode of administration can be left to the discretion of the practitioner, and depends in-part upon the site of the medical condition. In most instances, administration results in the release of any agent described herein into the bloodstream.

Any flagellin variant (and/or additional agents) described herein can be administered orally. Such flagellin variants (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment.

In one embodiment, any flagellin variant (and/or additional agents) described herein is formulated in accordance with routine procedures as a composition adapted for oral administration to humans. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any flagellin variant (and/or additional agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

The dosage of any flagellin variant (and/or additional agents) described herein as well as the dosing schedule can depend on various parameters, including, but not limited to, the disease being treated, the subject's general health, and the administering physician's discretion. Any agent described herein, can be administered prior to (e.g., about 5 minutes, about 15 minutes, about 30 minutes, about 45 minutes, about 1 hour, about 2 hours, about 4 hours, about 6 hours, about 12 hours, about 24 hours, about 48 hours, about 72 hours, about 96 hours, about 1 week, about 2 weeks, about 3 weeks, about 4 weeks, about 5 weeks, about 6 weeks, about 8 weeks, or about 12 weeks before), concurrently with, or subsequent to (e.g., about 5 minutes, about 15 minutes, about 30 minutes, about 45 minutes, about 1 hour, about 2 hours, about 4 hours, about 6 hours, about 12 hours, about 24 hours, about 48 hours, about 72 hours, about 96 hours, about 1 week, about 2 weeks, about 3 weeks, about 4 weeks, about 5 weeks, about 6 weeks, about 8 weeks, or about 12 weeks after) the administration of an additional therapeutic agent, to a subject in need thereof. In various embodiments any agent described herein is administered about 1 minute apart, about 10 minutes apart, about 30 minutes apart, less than about 1 hour apart, about 1 hour apart, about 1 hour to about 2 hours apart, about 2 hours to about 3 hours apart, about 3 hours to about 4 hours apart, about 4 hours to about 5 hours apart, about 5 hours to about 6 hours apart, about 6 hours to about 7 hours apart, about 7 hours to about 8 hours apart, about 8 hours to about 9 hours apart, about 9 hours to about 10 hours apart, about 10 hours to about 11 hours apart, about 11 hours to about 12 hours apart, no more than about 24 hours apart or no more than 48 hours apart.

The amount of any flagellin variant (and/or additional agents) described herein that is admixed with the carrier materials to produce a single dosage can vary depending upon the subject being treated and the particular mode of administration. In vitro or in vivo assays can be employed to help identify optimal dosage ranges.

In general, the doses that are useful are known to those in the art. For example, doses may be determined with reference Physicians' Desk Reference, 66th Edition, PDR Network; 2012 Edition (Dec. 27, 2011), the contents of which are incorporated by reference in its entirety.

The dosage of any flagellin variant (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

Generally, when orally administered to a mammal, the dosage of any flagellin variant (and/or additional agents) described herein may be about 0.001 mg/kg/day to about 100 mg/kg/day, about 0.01 mg/kg/day to about 50 mg/kg/day, or about 0.1 mg/kg/day to about 10 mg/kg/day. When orally administered to a human, the dosage of any agent described herein is normally about 0.001 mg to about 1000 mg per day, about 1 mg to about 600 mg per day, or about 5 mg to about 30 mg per day.

For administration of any flagellin variant (and/or additional agents) described herein by parenteral injection, the dosage is normally about 0.1 mg to about 250 mg per day, about 1 mg to about 20 mg per day, or about 3 mg to about 5 mg per day. Injections may be given up to four times daily. Generally, when orally or parenterally administered, the dosage of any agent described herein is normally about 0.1 mg to about 1500 mg per day, or about 0.5 mg to about 10 mg per day, or about 0.5 mg to about 5 mg per day. A dosage of up to about 3000 mg per day can be administered.

In another embodiment, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any flagellin variant (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any flagellin variant (and/or additional agents) described herein can, independently, be one to four times daily or one to four times per month or one to six times per year or once every two, three, four or five years. Administration can be for the duration of about one day or about one month, about two months, about three months, about six months, about one year, about two years, about three years, and may even be for the life of the subject. Chronic, long-term administration will be indicated in many cases. The dosage may be administered as a single dose or divided into multiple doses. In general, the desired dosage should be administered at set intervals for a prolonged period, usually at least over several weeks or months, although longer periods of administration of several months or years or more may be needed.

The dosage regimen utilizing any flagellin variant (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any flagellin variant (and/or additional agents) described herein can be administered in a single daily dose, or the total daily dosage can be administered in divided doses of two, three or four times daily. Furthermore, any flagellin variant (and/or additional agents) described herein can be administered continuously rather than intermittently throughout the dosage regimen.

### Combination Therapies and Conjugation

In some embodiments, the invention provides for flagellin variants and methods that further comprise administering an additional agent to a subject. In some embodiments, the invention pertains to co-administration and/or co-formulation. Any of the compositions described herein may be co-formulated and/or co-administered.

In some embodiments, any flagellin variant described herein acts synergistically when co-administered with another agent and is administered at doses that are lower than the doses commonly employed when such agents are used as monotherapy. In various embodiments, any agent referenced herein may be used in combination with any of the flagellin variants described herein.

Immune Checkpoint Inhibitor (CPI) Immunotherapy

The present invention provides, in part, pharmaceutical compositions, formulations, and uses of immune checkpoint inhibitor immunotherapies in conjunction with flagellin variant therapy. For example, in some embodiments, the flagellin variant is the mutated flagellin variant of the present invention (e.g., 491TEMX/SE-2/GP532). In some embodiments, the flagellin variant is entolimod.

Cancer immunotherapy involves the utilization of naturally derived or synthetically generated components to stimulate or enhance the immune system to fight cancer. Immune checkpoint inhibitor immunotherapies are effective in fighting cancer due to the priming and activation of the immune system in order to produce antitumor effects, often involving highly specific targeting. Along with the promise of cancer immunotherapy, there is the need to maintain the immune system's complex counterbalance between identification and eradication of foreign antigens and the processes necessary for suppressing an uncontrolled immune response. Despite important clinical benefits, checkpoint inhibition is associated with a unique spectrum of side effects, or immune-related adverse events, including, but not limited to, dermatologic, GI, hepatic, endocrine, and other less common inflammatory events. In various embodiments of the present invention, the CPI-mediated GI side effect is diarrhea and/or colitis. Generally, treatment of these moderate or severe immune checkpoint inhibitor immunotherapy-mediated side effects can require interruption of the checkpoint inhibitor immunotherapy and use of corticosteroid immunosuppression.

In some aspects, the present invention contemplates pharmaceutical compositions, formulations, and uses of immune checkpoint inhibitor immunotherapies in conjunction with flagellin variant therapy. Such CPIs can include, but are not limited to, one or more agents that modulate one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4). In some embodiments, the patient is undergoing therapy with an immune checkpoint inhibitor immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

In other aspects, the present invention contemplates methods for preventing CPI-mediated GI side effects by administering a combination of an IAP and a CPI selected from an agent that modulates one or more of PD-1, PD-L1, PD-L2, ICOS, ICOSL, and CTLA-4.

In some embodiments, the agent that modulates one or more of PD-1, PD-L1, PD-L2, ICOS, ICOSL, and CTLA-4 is an antibody or antibody format specific for one or more of PD-1, PD-L1, PD-L2, ICOS, ICOSL, and CTLA-4. In various embodiments, the antibody or antibody format specific for one or more of PD-1, PD-L1, PD-L2, ICOS, ICOSL, and CTLA-4 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

For example, in some embodiments, the present invention provides for a CPI that is an agent that modulates PD-1, wherein the agent is an antibody or antibody format specific for PD-1. In some embodiments, the antibody or antibody format specific for PD-1 is selected from Nivolumab, Pembrolizumab, and Pidilizumab. In other embodiments, the present invention provides for a CPI that is an agent that modulates PD-L1, wherein the agent is an antibody or antibody format specific for PD-L1. In some embodiments, the antibody or antibody format specific for PD-L1 is selected from BMS-936559, Atezolizumab, Avelumab and Durvalumab. In other embodiments, the present invention provides for a CPI that is an agent that modulates PD-L2, wherein the agent is an antibody or antibody format specific for PD-L2. In other embodiments, the present invention provides for a CPI that is an agent that modulates ICOS, wherein the agent is an antibody or antibody format specific for ICOS. In some embodiments, the antibody or antibody format specific for ICOS comprises JTX-2011. In other embodiments, the present invention provides for a CPI that is an agent that modulates ICOSL, wherein the agent is an antibody or antibody format specific for ICOSL. In other embodiments, the present invention provides for a CPI that is an agent that modulates CTLA-4, wherein the agent is an antibody or antibody format specific for CTLA-4. In some embodiments, the antibody or antibody format specific for CTLA-4 is selected from tremelimumab or Ipilimumab.

Chemotherapeutic Agents

In some embodiments, the present invention pertains to chemotherapeutic agents as additional agents.

Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; def of amine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In some embodiments, the flagellin variants (and/or additional agents) described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

In still other embodiments, the flagellin variants (and/or additional agents) described herein further comprise a cytotoxic agent, comprising, in exemplary embodiments, a toxin, a chemotherapeutic agent, a radioisotope, and an agent that causes apoptosis or cell death. Such agents may be conjugated to a composition described herein.

The flagellin variants (and/or additional agents) described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Exemplary cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-β1 antagonists, interleukins (e.g. IL-12 or IL-2), IL-12R antagonists, Toxin conjugated monoclonal antibodies, tumor antigen specific monoclonal antibodies, Erbitux, Avastin, Pertuzumab, anti-CD20 antibodies, Rituxan, ocrelizumab, ofatumumab, DXL625, HERCEPTIN®, or any combination thereof. Toxic enzymes from plants and bacteria such as ricin, diphtheria toxin and Pseudomonas toxin may be conjugated to the therapeutic agents (e.g. antibodies) to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the antibody, or binding fragments thereof, with or without the use of a complex-forming agent. Such radionuclides include beta-emitters such as Phosphorus-32, Scandium-47, Copper-67, Gallium-67, Yttrium-88, Yttrium-90, Iodine-125, Iodine-131, Samarium-153, Lutetium-177, Rhenium-186 or Rhenium-188, and alpha-emitters such as Astatine-211, Lead-212, Bismuth-212, Bismuth-213 or Actinium-225.

Exemplary detectable moieties further include, but are not limited to, horseradish peroxidase, acetylcholinesterase, alkaline phosphatase, beta-galactosidase and luciferase. Further exemplary fluorescent materials include, but are not limited to, rhodamine, fluorescein, fluorescein isothiocyanate, umbelliferone, dichlorotriazinylamine, phycoerythrin and dansyl chloride. Further exemplary chemiluminescent moieties include, but are not limited to, luminol. Further exemplary bioluminescent materials include, but are not limited to, luciferin and aequorin. Further exemplary radioactive materials include, but are not limited to, Iodine-125, Carbon-14, Sulfur-35, Tritium and Phosphorus-32.

In various embodiments, the additional agents of the present invention include one or more of blood products, colony stimulating factors, cytokines and/or growth factors, antibiotics, diluting and/or blocking agents, mobilizing or chelating agents, stem cell transplants, antioxidants or free radicals, and radioprotectants.

In some embodiments, the blood product is one or more of hematopoietic growth factors, such as filgrastim (e.g. NEUPOGEN), a granulocyte colony-stimulating factor (G-CSF), which may be optionally pegylated (e.g. NEULASTA); sargramostim (LEUKINE); and a granulocyte-macrophage colony-stimulating factor (GM-CSF) and a KSF.

In some embodiments, the additional agent is one or more cytokines and/or growth factors that may confer radioprotection by replenishing and/or protecting the radiosensitive stem cell populations. Radioprotection with minimal side effects may be achieved by the use of stem cell factor (SCF, c-kit ligand), Flt-3 ligand, and interleukin-1 beta. Protection may be achieved through induction of proliferation of stem cells (e.g. via all mentioned cytokines), and prevention of their apoptosis (e.g. via SCF). The treatment allows accumulation of leukocytes and their precursors prior to irradiation thus enabling quicker reconstitution of the immune system after irradiation. SCF efficiently rescues lethally irradiated mice with a dose modifying factor (DMF) in range 1.3-1.35 and is also effective against gastrointestinal syndrome. Flt-3 ligand also provides strong protection in mice and rabbits.

Several factors, while not cytokines by nature, stimulate the proliferation of the immunocytes and may be used in combination with the flagellin variants at the doses and regimens described herein. For example, 5-AED (5-androstenediol) is a steroid that stimulates the expression of cytokines and increases resistance to bacterial and viral infections. Synthetic compounds, such as ammonium tri-chloro(dioxoethylene-O,O′—) tellurate (AS-101), may also be used to induce secretion of numerous cytokines and for combination with the flagellin variants. Growth factors and cytokines may also be used to provide protection against the gastrointestinal syndrome. Keratinocyte growth factor (KGF) promotes proliferation and differentiation in the intestinal mucosa, and increases the post-irradiation cell survival in the intestinal crypts. Hematopoietic cytokine and radioprotectant SCF may also increase intestinal stem cell survival and associated short-term organism survival.

In certain embodiments, the flagellin variants may be added to a regimen of cytokines (e.g. for FILGRASTIM (G-CSF) 2.5-5 μg/kg/d QD s.c. (100-200 μg/m²/d); for SARGRAMOSTIM (GM-CSF) 5-10 μg/kg/d QD s.c. (200-400 μg/m²/d); and/or for PEGFILGRASTIM (pegG-CSF) 6 mg once s.c.).

In some embodiments, the antibiotic is one or more of an anti-bacterial (anti-gram positive and anti-gram negative agents), and/or anti-fungal, and/or anti-viral agent. By way of non-limiting example, in some embodiments, the antibiotic may be a quinolone, e.g. ciprofloxacin, levofloxacin, a third- or fourth-generation cephalosporin with pseudomonal coverage: e.g., cefepime, ceftazidime, or an aminoglycoside: e.g. gentamicin, amikacin, penicillin or amoxicillin, acyclovir, vanomycin. In various embodiments, the antibiotic targets Pseudomonas aeruginosa.

In some embodiments, the additional agent is a diluting and/or blocking agents. For example, stable iodide compounds may be used (e.g. liquid (ThyroShield) and the tablet (losat) KI (NUKEPILLS), Rad Block, I.A.A.A.M., No-Rad, Life Extension (LEF), K14U, NukeProtect, ProKI)). A 130 mg dose of daily of oral potassium iodide (KI) may be used in conjunction with the flagellin variants.

In some embodiments, the additional agent is a mobilizing or chelating agent. Illustrative mobilizing agents include propylthiouracil and methimazole, with may reduce the thyroid's retention of radioactive compounds. Further the flagellin variants can be used alongside increasing oral fluids to a human patient to promote excretion. Illustrative chelating agents are water soluble and excreted in urine. Illustrative chelating agents include DTPA and EDTA. Dimercaprol forms stable chelates with mercury, lead, arsenic, gold, bismuth, chromium, and nickel and therefore may be considered for the treatment of internal contamination with the radioisotopes of these elements. Penicillamine chelates copper, iron, mercury, lead, gold, and possibly other heavy metals.

In some embodiments, the additional agent is a stem cell transplant (e.g. bone marrow transplant, PBSCT, MSCT). In some embodiments the stem cell transpant is Remestemcel-L (Osiris) of CLT-008 (Cellerant).

In some embodiments, the additional agent is an antioxidant or free radical. Antioxidants and free radical scavengers that may be used in the practice of the invention include, but are not limited to, thiols, such as cysteine, cysteamine, glutathione and bilirubin; amifostine (WR-2721); vitamin A; vitamin C; vitamin E; and flavonoids such as Indian holy basil (Ocimum sanctum), orientin and vicenin.

In some embodiments, the additional agent may be a radioprotectant e.g. an antioxidant (e.g. amifostine and vitamin E, gamma tocotrienol (a vitamin-E moiety), and genistein (a soy byproduct)), a cytokine (e.g. a stem cell factor), a growth factor (e.g. keratinocyte growth factor), a steroid (e.g. 5-androstenediol), ammonium trichloro(dioxoethylene-O,O′)tellurate, thyroid protecting agents (e.g. Potassium iodide (KI) or potassium iodate (KIO₃) (e.g. liquid (ThyroShield) and the tablet (losat) KI (NUKEPILLS), Rad Block, I.A.A.A.M., No-Rad, Life Extension (LEF), K14U, NukeProtect, ProKI)), anti-nausea agents, anti-diarrhea agents, antiemetics ((e.g. oral prophylactic antiemetics) such as granisetron (KYTRIL), ondansetron (ZOFRAN), and 5-HT3 blockers with or without dexamethasone), analgesics, anxiolytics, sedatives, cytokine therapy, and antibiotics.

Gastric lavage and emetics, which can be used as additional agents, can be used to empty the stomach promptly and completely after the ingestion of poisonous materials. Purgatives, laxatives, and enemas, which also can be used as additional agents, can reduce the residence time of radioactive materials in the colon. Further additional agents include ion exchange resins which may limit gastrointestinal uptake of ingested or inhaled radionuclides, ferric ferrocyanide (Prussian blue) and alginates, which have been used in humans to accelerate fecal excretion of cesium-137.

In still other embodiments, the additional agent may be an agent used to treat radiation-related disorders, such as, for example, 5-AED (Humanetics), Ex-RAD (Onconova), Beclometasone Dipropionate (Soligenix), detoxified endotoxin, EA-230 (Exponential Biotherapies), ON-01210.Na (Onconova), Sothrombomodulin alfa (PAION), Remestemcel-L (Osiris), BIO-100, BIO-200, BIO-300, BIO-400, BIO-500 (Humanetics), CLT-008 (Cellerant), EDL-2000 (RxBio), Homspera (ImmuneRegen), MnDTEIP (Aeolus Pharmaceuticals), RLIP-76 (Terapio), and RX-100 and RX 101 (RxBio).

Further, in some embodiments, the flagellin variants (and/or additional agents) can be used in combination with shielding; reduction of radiation exposure time; and use of agents to reduce body exposure (e.g. uses of gloves, face mask, hood, protective clothing (e.g. anticontamination suits such as TYVEK ANTI-C SUITS or MOPP-4)).

### Viral Vectors Encoding Therapeutic Agents and Cells Expressing Same

In various embodiments, the flagellin variants (and/or additional agents) of the present invention is expressed by viral vectors and transformed cells. For example, the viral vectors and transformed human cells described herein may express the present compositions. In an embodiment, the viral vector or human cells expressing the therapeutic agent are capable of expressing the agent proximal to a tumor. The cells can be modified in vivo, or alternatively cells modified ex vivo can be administered to a patient by a variety of methods, such as by injection.

In one embodiment, the cell is a tumor cell. For ex vivo transformation, such tumor cells can be irradiated to eliminate the ability of the cell to replicate, as known in the art, while maintaining the transient expression of the therapeutic agent after administration. For in vivo transformation, non-integrative expression vectors may be preferred.

In certain embodiments, the tumor cell is autologous or endogenous. In the former instance, the tumor cell is taken from a patient, transfected or transduced with a construct encoding the therapeutic agent and re-introduced to the patient, for example after irradiation. In the latter instance, the tumor cell is transformed in vivo by local administration of an appropriate construct as described herein.

In an alternative embodiment, the modified tumor cell is allogeneic. The allogeneic tumor cell thus can be maintained in a cell line. In this instance, the tumor cell can be selected from the cell line, irradiated, and introduced to the patent.

Modified human cells capable of producing the flagellin variants (and/or additional agents) can be made by transfecting or transducing the cells with an expression vector encoding the therapeutic agent. Expression vectors for the expression of the flagellin variants (and/or additional agents), or a combination of therapeutic agents can be made by methods well known in the art.

In various embodiments, the flagellin variants (and/or additional agents) can be administered to a patient in the form of one or more nucleic acid construct.

In one embodiment, the construct comprises a retroviral vector. Retroviral vectors are capable of permanently integrating DNA encoding flagellin variants (and/or additional agents) into the cell genome. Thus, in the case of ex vivo manipulation of autologous or allogeneic cells, stable cell lines that constitutively produce the flagellin variants (and/or additional agents) can be prepared. In an embodiment, the cells are irradiated prior to administration to a patient. The irradiated cells produce the flagellin variants (and/or additional agents) for a limited period of time.

In one embodiment, the expression construct comprises an SFV vector, which demonstrates high levels of transient expression in mammalian cells. The SFV vector is described, for example, in Lundstrom, Expert Opin. Biol. Ther. 3:771-777 (2003), incorporated herein by reference in its entirety. Thus, in the case of in vivo manipulation of endogenous cells in a patient, transient expression of high levels of the flagellin variants (and/or additional agents) can be accomplished.

Systems capable of expressing recombinant protein in vivo are known in the art. By way of example, the system can use the 2A mediated antibody expression system disclosed in Fang et al., Nature Biotech. 23(5): 584-590 (2005) and U.S. Patent Publication No. 2005/0003506, the disclosures of which are expressly incorporated by reference herein in their entirety. Other systems known in the art are contemplated, and can also be adapted to produce the flagellin variants (and/or additional agents) in vivo as described herein.

In various embodiments, administration of the flagellin variant (and/or additional agents) expressing cells disclosed herein or the agents of the invention disclosed herein can be combined with administration of cytokines that stimulate antigen-presenting cells such as granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interferon, etc., or cellular vaccines capable of expressing such cytokines. In some embodiments, the flagellin variant (and/or additional agents) expressing cells are further modified to express such cytokines. Additional proteins and/or cytokines known to enhance T cell proliferation and secretion, such as IL-1, IL-2, B7, anti-CD3 and anti-CD28 can be employed simultaneously or sequentially with the flagellin variants (and/or additional agents) of the invention to augment the immune response, and/or stimulate co-stimulatory pathways and/or induce activation/proliferation of effector T cells.

### Vectors and Methods of Transformation

Expression vectors encoding the flagellin variants (and/or additional agents) may be viral or non-viral. Viral vectors are preferred for use in vivo. Expression vectors of the invention comprise a nucleic acid encoding the flagellin variants (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid.

An expression control region of an expression vector of the invention is capable of expressing operably linked encoding nucleic acid in a human cell. In an embodiment, the cell is a tumor cell. In another embodiment, the cell is a non-tumor cell.

In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In an embodiment, the present invention contemplates the use of inducible promoters capable of effecting high level of expression transiently in response to a cue. When in the proximity of a tumor cell, a cell transformed with an expression vector for the flagellin variants (and/or additional agents) comprising such an expression control sequence is induced to transiently produce a high level of the agent by exposing the transformed cell to an appropriate cue. Exemplary inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a B7-H4 ligand coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also preferred for in vivo transduction. In some situations it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al, Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003).

### Viral Vectors

In one aspect, the invention provides expression vectors for the expression of the flagellin variants (and/or additional agents) that are viral vectors. Many viral vectors useful for gene therapy are known (see, e.g., Lundstrom, Trends Biotechnol., 21: 1 17, 122, 2003.

Exemplary viral vectors include those selected from Antiviruses (LV), retroviruses (RV), adenoviruses (AV), adeno-associated viruses (AAV), and alphaviruses, though other viral vectors may also be used. For in vivo uses, viral vectors that do not integrate into the host genome are preferred, such as a viruses and adenoviruses, with a viruses being especially preferred. Exemplary types of a viruses include Sindbis virus, Venezuelan equine encephalitis (VEE) virus, and Semliki Forest virus (SFV), with SFV being especially preferred. For in vitro uses, viral vectors that integrate into the host genome are preferred, such as retroviruses, AAV, and alphaviruses.

In an embodiment, the viral vector provides for transient high level expression in a transduced human cell.

In one embodiment, the viral vector does not provide for integration of the flagellin variant (and/or additional agents) encoding nucleic acid into the genome of a transduced human cell.

In another embodiment, the viral vector provides for integration of the flagellin variants (and/or additional agents) encoding nucleic acid into the genome of a transduced human cell.

In one embodiment, the invention provides methods of transducing a human cell in vivo, comprising contacting a solid tumor in vivo with a viral vector of the invention.

In another embodiment, the invention provides methods of transducing a human cell ex vivo, comprising contacting a human cell ex vivo with the viral vector of the invention. In one embodiment, the human cell is a tumor cell. In one embodiment, the human cell is allogeneic. In one embodiment, the tumor cell is derived from the patient. In one embodiment, the human cell is a non-tumor cell, such as, e.g., an antigen presenting cell (APC), or a T cell.

Virus particle coats may be modified to alter specificity and improve cell/tissue targeting, as is well known in the art. Viral vectors may also be delivered in other vehicles, for example, liposomes. Liposomes may also have targeting moieties attached to their surface to improve cell/tissue targeting.

In some embodiments, the present invention provides human cells expressing the therapeutic agent of the invention. In various embodiments, the human cells express the agent proximal to a tumor cell of, for example, a patient.

### Diagnostic and Predictive Methods

In some aspects, the invention provides a method for identifying a subject who may respond to treatment with a TLR5 agonist. In some embodiments, the present invention provides a method of determining if a patient's tumor expresses TLR5.

TLR5 expression may be a predictive marker for determining the grade and/or progression of a patient's tumor or dysplasia. In some embodiments, the flagellin variants (and/or additional agents) described herein are useful in determining a tumor grade and/or stage of a particular cancer.

Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade may vary with each type of cancer and are known in the art.

Histologic grade, also called differentiation, refers to how much the tumor cells resemble normal cells of the same tissue type. Nuclear grade refers to the size and shape of the nucleus in tumor cells and the percentage of tumor cells that are dividing.

Based on the microscopic appearance of cancer cells, pathologists commonly describe tumor grade by four degrees of severity: Grades 1, 2, 3, and 4. The cells of Grade 1 tumors resemble normal cells, and tend to grow and multiply slowly. Grade 1 tumors are generally considered the least aggressive in behavior. Conversely, the cells of Grade 3 or Grade 4 tumors do not look like normal cells of the same type. Grade 3 and 4 tumors tend to grow rapidly and spread faster than tumors with a lower grade. The American Joint Committee on Cancer recommends the following guidelines for grading tumors: GX-grade cannot be assessed (Undetermined grade); G1-well-differentiated (Low grade); G2-moderately differentiated (Intermediate grade); G3-poorly differentiated (High grade); and G4-undifferentiated (High grade).

Grading systems are different for each type of cancer. For example, pathologists use the Gleason system to describe the degree of differentiation of prostate cancer cells. The Gleason system uses scores ranging from Grade 2 to Grade 10. Lower Gleason scores describe well-differentiated, less aggressive tumors. Higher scores describe poorly differentiated, more aggressive tumors. Other grading systems include, for example, the Bloom-Richardson system for breast cancer and the Fuhrman system for kidney cancer.

Cancer survival rates or survival statistics may refer to the percentage of people who survive a certain type of cancer for a specific amount of time. Cancer statistics often use an overall five-year survival rate. For example the overall five-year survival rate for bladder cancer is 80 percent, i.e. 80 of every 100 of people diagnosed with bladder cancer were living five years after diagnosis and 20 out of every 100 died within five years of a bladder cancer diagnosis. Other types of survival rates may be used, for example: disease-free survival rate (number of people with cancer who achieve remission) and progression-free survival rate. (number of people who still have cancer, but their disease is not progressing).

In some embodiments, the flagellin variants (and/or additional agents) described herein are useful in establishing a tumor grade for the purposes of diagnosis or prognosis of a particular cancer, including prognosing the survival rate, disease-free survival rate and/or progression-free survival rate prior to, during and/or after administration of a flagellin variant (and/or additional agents) disclosed herein and/or prior to, during and/or after administration of an anti-cancer agent or therapy.

In some embodiments, the flagellin variants (and/or additional agents) described herein are used as part of a method of scoring tumor grades to assist in the selection and/or predict the outcome of treatment. For example, the flagellin variants (and/or additional agents) described herein may be used to diagnose or identify the cancer from a patient as stage I (e.g. not locally advanced) predicting the need for less aggressive treatment. Alternatively, the therapeutic agent described herein may be used to diagnose or identify the cancer from a patient as stage II or III, (e.g. the cancer may be locally advanced) predicting the need for more aggressive treatment. Similarly, the flagellin variants (and/or additional agents) described herein may be used to diagnose or identify the cancer from a patient as stage IV, or is metastatic, predicting the need for very aggressive treatment.

In some embodiments, the cancer is non-resectable. A non-resectable cancer is a malignancy which cannot be surgically removed, due either to the number of metastatic foci, or because it is in a surgical danger zone. In some embodiments, the therapeutic agent described herein is used as part of a method of treating tumors to assist in selecting the nature and/or timing/administration of treatment including, for example, administering anti-cancer agents which reduce tumor volume, prior to chemotherapeutic and/or radiation treatment, and/or increase or decrease the dose of chemotherapy or radiation administered to a patient.

In some embodiments, the cancer is multidrug resistant. For example, the patient may have undergone one or more cycles of chemotherapy, without substantial response. Alternatively or in addition, the tumor has one or more markers of multidrug resistance. Thus, as used herein, the term multidrug resistant means a cancer exhibiting non-responsiveness to at least one cycle of combination chemotherapy, or alternatively, has scored (diagnostically) as resistant to at least two of (including comparable agent to) docetaxel, paclitaxel, doxorubicin, epirubicin, carboplatin, cisplatin, vinblastine, vincristine, oxaliplatin, carmustine, fluorouracil, gemcitabine, cyclophosphamide, ifosfamide, topotecan, erlotinib, etoposide, and mitomycin. In some embodiments, the therapeutic agents described herein are useful in establishing whether the tumor is responsive to one or more chemotherapeutics, radiation therapy and/or other anti-cancer therapy.

In other embodiments, the cancer is a recurrence following conventional chemotherapy of an initial cancer. Often, recurrent cancer has developed drug resistance, and thus is particularly difficult to treat and often comes with a poor prognosis for survival.

In some embodiments, the flagellin variants (and/or additional agents) described herein are used as part of a method of tumor evaluation which takes the place of a performance status. Performance status can be quantified using any system and methods for scoring a patient's performance status which are known in the art. The measure is often used to determine whether a patient can receive chemotherapy, dose adjustment, and/or to determine intensity of palliative care. There are various scoring systems, including the Karnofsky score and the Zubrod score. Parallel scoring systems include the Global Assessment of Functioning (GAF) score, which has been incorporated as the fifth axis of the Diagnostic and Statistical Manual (DSM) of psychiatry.

Higher performance status (e.g., at least about 80%, or at least about 70% using the Karnofsky scoring system) may indicate treatment to prevent progression of the disease state, and enhance the patient's ability to accept chemotherapy and/or radiation treatment. For example, when the therapeutic agent described herein indicates higher performance status, the patient is ambulatory and capable of self care. In other embodiments, when the therapeutic agent described herein indicates a low performance status (e.g., less than about 50%, less than about 30%, or less than about 20% using the Karnofsky scoring system), the patient is largely confined to bed or chair and is disabled even for self-care.

The Karnofsky score runs from 100 to 0, where 100 is “perfect” health and 0 is death. The score may be employed at intervals of 10, where: about 100% is normal, no complaints, no signs of disease; about 90% is capable of normal activity, few symptoms or signs of disease, about 80% is normal activity with some difficulty, some symptoms or signs; about 70% is caring for self, not capable of normal activity or work; about 60% is requiring some help, can take care of most personal requirements; about 50% requires help often, requires frequent medical care; about 40% is disabled, requires special care and help; about 30% is severely disabled, hospital admission indicated but no risk of death; about 20% is very ill, urgently requiring admission, requires supportive measures or treatment; and about 10% is moribund, rapidly progressive fatal disease processes.

The Zubrod scoring system for performance status includes: 0, fully active, able to carry on all pre-disease performance without restriction; 1, restricted in physically strenuous activity but ambulatory and able to carry out work of a light or sedentary nature, e.g., light house work, office work; 2, ambulatory and capable of all self-care but unable to carry out any work activities, up and about more than about 50% of waking hours; 3, capable of only limited self-care, confined to bed or chair more than about 50% of waking hours; 4, completely disabled, cannot carry on any self-care, totally confined to bed or chair; 5, dead.

In some embodiments, histological samples of tumors are graded using the therapeutic agent described herein according to Elston & Ellis, Histopathology, 1991, 19:403-10, which is hereby incorporated by reference in its entirety. In some embodiments, the therapeutic agent described herein is useful in establishing a tumor grade for the purposes of diagnosis or prognosis of a particular cancer.

In some embodiments, the flagellin variants (and/or additional agents) described herein are useful for evaluating a subject and/or a specimen from a subject (e.g. a cancer patient). In some embodiments, evaluation is one or more of diagnosis, prognosis, and/or response to treatment.

Diagnosis refers to the process of attempting to determine or identify a possible disease or disorder, such as, for example, cancer. Prognosis refers to the predicting of a likely outcome of a disease or disorder, such as, for example, cancer. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, pathological complete response, survival, and probability of recurrence.

In various embodiments, the diagnostic and predictive methods described herein comprise evaluating a presence, absence, or level of a protein. In another embodiment, the methods described herein comprise evaluating a presence, absence, or level of expression of a nucleic acid. The compositions described herein may be used for these measurements. For example, in some embodiments, the methods described herein comprise contacting a specimen of the tumor or cells cultured from the tumor with a therapeutic agent as described herein.

In some embodiments, the present invention includes the measurement of a tumor specimen, including biopsy or surgical specimen samples. In some embodiments, the biopsy is a human biopsy. In various embodiments, the biopsy is any one of a frozen tumor tissue specimen, cultured cells, circulating tumor cells, and a formalin-fixed paraffin-embedded tumor tissue specimen. In some embodiments, the tumor specimen may be a biopsy sample, such as a frozen tumor tissue (cryosection) specimen. As is known in the art, a cryosection may employ a cryostat, which comprises a microtome inside a freezer. The surgical specimen is placed on a metal tissue disc which is then secured in a chuck and frozen rapidly to about −20° C. to about −30° C. The specimen is embedded in a gel like medium consisting of, for example, poly ethylene glycol and polyvinyl alcohol. The frozen tissue is cut frozen with the microtome portion of the cryostat, and the section is optionally picked up on a glass slide and stained. In some embodiments, the tumor specimen may be a biopsy sample, such as cultured cells. These cells may be processed using the usual cell culture techniques that are known in the art. These cells may be circulating tumor cells. In some embodiments, the tumor specimen may be a biopsy sample, such as a formalin-fixed paraffin-embedded (FFPE) tumor tissue specimen. As is known in the art, a biopsy specimen may be placed in a container with formalin (a mixture of water and formaldehyde) or some other fluid to preserve it. The tissue sample may be placed into a mold with hot paraffin wax. The wax cools to form a solid block that protects the tissue. This paraffin wax block with the embedded tissue is placed on a microtome, which cuts very thin slices of the tissue. In certain embodiments, the tumor specimen contains less than about 100 mg of tissue, or in certain embodiments, contains about 50 mg of tissue or less. The tumor specimen (or biopsy) may contain from about 20 mg to about 50 mgs of tissue, such as about 35 mg of tissue. The tissue may be obtained, for example, as one or more (e.g., 1, 2, 3, 4, or 5) needle biopsies (e.g., using a 14-gauge needle or other suitable size). In some embodiments, the biopsy is a fine-needle aspiration in which a long, thin needle is inserted into a suspicious area and a syringe is used to draw out fluid and cells for analysis. In some embodiments, the biopsy is a core needle biopsy in which a large needle with a cutting tip is used during core needle biopsy to draw a column of tissue out of a suspicious area. In some embodiments, the biopsy is a vacuum-assisted biopsy in which a suction device increases the amount of fluid and cells that is extracted through the needle. In some embodiments, the biopsy is an image-guided biopsy in which a needle biopsy is combined with an imaging procedure, such as, for example, X ray, computerized tomography (CT), magnetic resonance imaging (MRI) or ultrasound. In other embodiments, the sample may be obtained via a device such as the MAMMOTOME® biopsy system, which is a laser guided, vacuum-assisted biopsy system for breast biopsy.

In some embodiments, the diagnostic and predictive methods and/or evaluation may direct treatment (including treatment with the therapeutic agents described herein). In one embodiment, the evaluation may direct the use or withholding of adjuvant therapy after resection. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the therapeutic agents described herein are used as an adjuvant therapy in the treatment of a cancer. In some embodiments, the therapeutic agents described herein are used as the sole adjuvant therapy in the treatment of a cancer. In some embodiments, the therapeutic agents described herein are withheld as an adjuvant therapy in the treatment of a cancer. For example, if a patient is unlikely to respond to a therapeutic agent described herein or will have a minimal response, treatment may not be administered in the interest of quality of life and to avoid unnecessary toxicity from ineffective chemotherapies. In such cases, palliative care may be used.

In some embodiments the therapeutic agents described herein are administered as a neoadjuvant therapy prior to resection. In certain embodiments, neoadjuvant therapy refers to therapy to shrink and/or downgrade the tumor prior to any surgery. In some embodiments, neoadjuvant therapy means chemotherapy administered to cancer patients prior to surgery. In some embodiments, neoadjuvant therapy means a therapeutic agent described herein is administered to cancer patients prior to surgery. Types of cancers for which neoadjuvant chemotherapy is commonly considered include, for example, breast, colorectal, ovarian, cervical, bladder, and lung. In some embodiments, the therapeutic agents described herein are used as a neoadjuvant therapy in the treatment of a cancer. In some embodiments, the use is prior to resection. In some embodiments, the therapeutic agents described herein are withheld as a neoadjuvant therapy in the treatment of a cancer. For example, if a patient is unlikely to respond to a therapeutic agent described herein or will have a minimal response, treatment may not be administered in the interest of quality of life and to avoid unnecessary toxicity from ineffective chemotherapies. In such cases, palliative care may be used.

Subjects and/or Animals

In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP).

In some embodiments, the subject and/or animal is a human. In some embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

Kits

The invention provides kits that can simplify the administration of any agent described herein. An exemplary kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

### Definitions

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents (e.g. flagellin variants (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a patient suffering from cancer provides a therapeutic benefit not only when the underlying condition is eradicated or ameliorated, but also when the patient reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the 1050 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

In certain embodiments, a pharmacologically effective amount that will treat cancer will modulate the symptoms typically by at least about 10%, at least about 20%, at least about 30%, at least about 40%, or at least about 50%. In exemplary embodiments, such modulations will result in, for example, statistically significant and quantifiable changes in the numbers of cancerous cells.

This invention is further illustrated by the following non-limiting examples.

### EXAMPLES

### Example 1: Engineering of Improved Flagellin Variants Relative to CBLB502 and 33MX

a. CD4⁺ T Cell Epitope Mapping:

80 peptides derived from a flagellin derivative, each 15 amino acids in length, were analyzed for the presence of CD4⁺ T cell epitopes using EpiScreen™ T cell epitope mapping technology. The peptides were synthesized and tested against peripheral blood mononuclear cells (PBMC) from a cohort of 50 healthy human donors. CD4⁺ T cell responses against individual peptides were measured using proliferation assays (³[H]-thymidine incorporation). Positive responses were observed to five peptides containing Human Leukocyte Antigen-DR isotype (HLA-DR) restricted major histocompatibility complex (MHC) class II binding motifs.

In particular, a pre-clinical, ex vivo T cell assay was used to provide a prediction of T cell immunogenicity by identifying linear T cell epitopes present in protein sequences. Synthetic overlapping peptides of 15 amino acids in length were individually tested in sextuplicate cultures against a cohort of community blood donors (50 healthy donors) carefully selected based on MHC haplotypes to provide a quantitative analysis of T cell epitopes present in protein sequences, both the location of epitopes as well as their relative potency. This analysis provides a comparison of protein variants for potential of inducing immune responses in vivo.

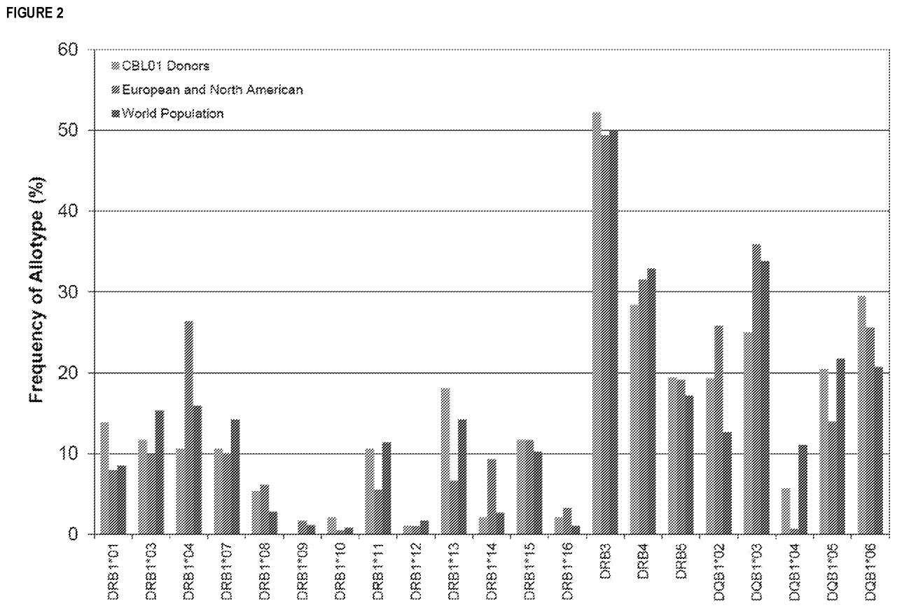

Donor selection was carried out when PBMC were isolated from healthy community donor buffy coats (from blood drawn within 24 hours) obtained under consent from commercial vendors. Cells were separated by Lymphoprep (Axis-shield, Dundee, UK) density centrifugation and CD8⁺ T cells were depleted using CD8⁺ RosetteSep™ (StemCell Technologies Inc, London, UK). Donors were characterized by identifying HLA-DR haplotypes using the HISTO Spot SSO HLA typing method (MC Diagnostics, St. Asaph, UK). T cell responses to a control neoantigen protein (keyhole limpet haemocyanin (KLH), Sigma, Poole, UK) and control peptides derived from Influenza virus (IFV) (C32) and Epstein Barr virus (EBV) (C3) were also determined. PBMC were then frozen and stored in liquid nitrogen until required. A cohort of 50 donors was selected to best represent the number and frequency of HLA-DR and DQ allotypes expressed in the world population. Analysis of the allotypes expressed revealed that the cohort covered all major HLA-DR and DQ allotypes. FIG. 2 shows a comparison of the distribution and frequency of MHC class II haplotypes expressed in the world, European and North American populations against the selected donor cohort.

Proliferation assays were then performed. PBMC from each donor were thawed, counted and viability was assessed. Cells were revived in room temperature AIM V® culture medium (Invitrogen, Paisley, UK) before adjusting the cell density to 2.5-3.5×10⁶ PBMC/ml (proliferation cell stock). Peptides were synthesized on a 1-3 mg scale with free N-terminal amine and C-terminal carboxylic acid. Peptides were dissolved in dimethyl sulphoxide (DMSO) to a concentration of 10 mM and peptide culture stocks prepared by diluting into AIM V® culture medium to a final concentration of 5 μM in each well. For each peptide and each donor, sextuplicate cultures were established in a flat bottomed 96 well plate. Both positive and negative control cultures were also tested in sextuplicate. For each donor, three controls (KLH protein (final assay concentration 0.3 μM) and peptides derived from IFV and EBV) were also included. For a positive control, PHA (Sigma, Poole, UK) was used at a final concentration of 2.5 μg/ml. Cultures were incubated for a total of 6 days before adding 0.75 μCi ³[H]-thymidine (Perkin Elmer®, Beaconsfield, UK) to each well. Cultures were incubated for a further 18 hours before harvesting onto filter mats using a TomTec Mach III cell harvester. Counts per minute (CPM) for each well were determined by Meltilex™ (Perkin Elmer®, Beaconsfield, UK) scintillation counting on a Microplate Beta Counter (Perkin Elmer®, Beaconsfield, UK) in paralux, low background counting mode.

In addition, all peptides were screened for endotoxin contamination using LAL Chromogenic Endotoxin Quantitation Kit (Pierce (Perbio), Cramlington, UK). A formulated standard curve was used to determine the endotoxin concentration of each sample. All peptides were found to contain levels of endotoxin that were below acceptable limits (<5 EU/mg).

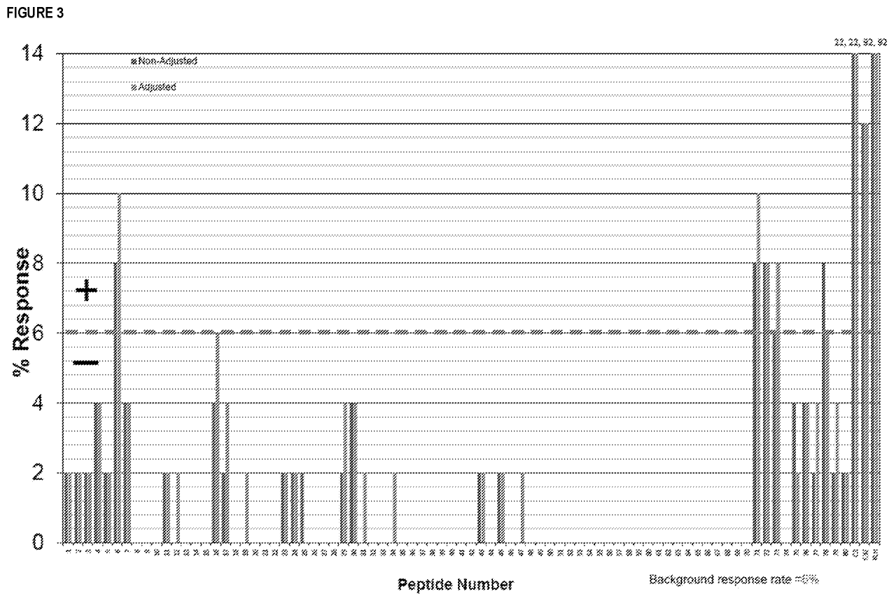

The proliferation assays were then analyzed statistically, where positive T cell responses were defined by donors whose PBMC produced a significant (p<0.05) response with a SI 2.00 to any given peptide. As shown in FIG. 3, T cell epitopes were identified by calculating the average frequency of positive responses to all peptides in the study plus 1.5×SD (termed “background response threshold”). FIG. 3 shows the frequency of positive donor responses to each peptide in the T cell proliferation assay. Peptides were considered to contain a T cell epitope if they induced positive T cell proliferation responses (SI≥2.00, p<0.05, including borderline responses SI≥1.90, p<0.05) in 3 or more donors in both the non-adjusted and adjusted data sets (≥6% of the donor cohort).

Any peptide that induced proliferative responses above this threshold was considered to contain a T cell epitope. A total of five peptides induced positive responses at or above the 6% cut off in both the non-adjusted and adjusted data sets. The magnitude and frequency of responses to the peptides suggested one weak, one moderate, and one strong T cell epitope.

b. Design of Epitope Variants of a Flagellin Derivative:

A series of epitope variants of a flagellin derivative were designed so that the immunogenic regions, previously identified by T cell epitope mapping, were eliminated while retaining binding affinity and other structural properties of the flagellin derivative.

De-immunization via site-directed mutagenesis using oligonucleotide primers and/or synthetic DNA, and/or deletions was determined by selecting specific amino acids in various epitopes under a number of complex considerations, including but not limited to, biophysical and biochemical data such as constraints on modification of the reference flagellin structure taking into account secondary and tertiary protein structures, as well as potential interactions of amino acid side chains with the core of the protein and with the receptor, TLR5. Specific amino acid changes and/or deletions within the sequences of Epitopes 1, 2, and 3 were evaluated in order to determine which mutations would reduce or eliminate MHC class II binding in order to remove associated T cell epitopes.

The inventors found that mutagenesis experimentation with the aim of reducing or eliminating de novo immunogenicity associated with T cell epitopes had to be balanced with adequate activity of the flagellin variant. Indeed, it was found that predictions based on structure considerations were not completely suitable. As a result, trial and error experimentation ensued in order to provide for a variant with balance of reduced immunogenicity and activity. A brief summary of the experimentation of mutations and epitope mapping is provided in Table 1 below.

TABLE 1
Flagellin variant (33MX) epitope mapping and de-immunization

| | Protein | | | EC50, | EC59(TEM)/ | SEQ ID |
|---|---|---|---|---|---|---|
| # | ID | Mutations | Position | ng/ml | EC50(33MX) | NO: |
| 1 | TEM1-AD | F22A; T23D | Epitope 1 | 0.089 | 2.8 | 7 |
| 2 | TEM1-SD | F22S; T23D | Epitope 1 | 0.117 | 2.8 | 8 |
| 3 | TEM1-TD | F22T; T23D | Epitope 1 | 0.148 | 3.2 | 9 |
| 4 | TEM1-DD | T23D; S24D | Epitope 1 | 0.171 | 4.0 | 10 |
| 5 | TEM1-49A | I18A | Epitope 1 | 0.113 | 1.4 | 11 |
| 6 | TEM1-53A | F22A | Epitope 1 | 0.112 | 1.4 | 12 |
| 7 | TEM1-54D | T23D | Epitope 1 | 0.146 | 1.7 | 13 |
| 8 | TEM1-49E | I18E | Epitope 1 | 0.147 | 1.6 | 14 |
| 9 | TEM1-49T | I18T | Epitope 1 | 0.137 | 1.5 | 15 |
| 10 | TEM1-58E | K27E | Epitope 1 | 0.181 | 2.0 | 16 |
| 11 | TEM2-ADT | I215A; L216D; | Epitope 2 | >10 | ND | 17 |
| | | V223T | | | | |
| 12 | TEM2-ADD | I215A; L216D; | Epitope 2 | >10 | ND | 18 |
| | | V224D | | | | |
| 13 | TEM2-ANT | I215A; L216N; | Epitope 2 | >10 | ND | 19 |
| | | V223T | | | | |
| 14 | TEM2-AND | I215A; L216N; | Epitope 2 | >10 | ND | 20 |
| | | V224D | | | | |
| 15 | TEM2-AST | I215A; L216S; | Epitope 2 | >10 | ND | 21 |
| | | V223T | | | | |
| 16 | TEM2-ASD | I215A; L216S; | Epitope 2 | >10 | ND | 22 |
| | | V224D | | | | |
| 17 | TEM2-480A | I215A | Epitope 2 | 0.662 | 7.5 | 23 |
| 18 | TEM2-481A | L216A | Epitope 2 | 0.836 | 9.7 | 24 |
| 19 | TEM2-488T | V223T | Epitope 2 | 0.103 | 1.2 | 25 |
| 20 | TEM2-481D | L216D | Epitope 2 | >10 | ND | 26 |
| 21 | TEM2-481H | L216H | Epitope 2 | >10 | ND | 27 |
| 22 | TEM2-482D | Q217D | Epitope 2 | 0.441 | 5.8 | 28 |
| 23 | TEM2-486D | T221D | Epitope 2 | 0.479 | 6.3 | 29 |
| 24 | TEM2-K1D | I215K; L216D | Epitope 2 | >10 | ND | 30 |
| 25 | TEM2-D6D | Q217D; T221D | Epitope 2 | >2.5 | ND | 31 |
| 26 | TEM2492DG | T221D; A182G | Epitope 2 | >2.5 | ND | 32 |
| 27 | TEM3-AS | V234A; L235S | Epitope 3 | 0.397 | 9.1 | 33 |
| 28 | TEM3-TA | V234T; L235A | Epitope 3 | 0.391 | 8.3 | 34 |
| 29 | 33TEMX | I18A; F22A; | Epitopes 1 and 2; | 0.686 | 10.4 | 35 |
| | | Q217D; V223T | Epitope 3 eliminated | | | |
| | | | (N-teminal tag #1) | | | |
| 30 | 33TXT | I18A; F22A; | Epitopes 1 and 2; | 0.091 | 1.4 | 36 |
| | | Q217D; V223T | Epitope 3 eliminated | | | |
| | | | (N-teminal tag #2) | | | |
| 31 | 33TXH | I18A; F22A; | Epitopes 1 and 2; | 0.620 | 11.4 | 37 |
| | | Q217D; V223T | Epitope 3 eliminated | | | |
| | | | (N-teminal tag #3) | | | |
| 32 | 33TXL | I18A; F22A; | Epitopes 1 and 2; | 0.533 | 9.9 | 38 |
| | | Q217D; V223T | Epitope 3 eliminated | | | |
| | | | (N-teminal tag #4) | | | |
| 33 | 33TX2 | I18A; F22A; | Epitopes 1 and 2; | 0.254 | 4.7 | 39 |
| | | Q217D; V223T | Epitope 3 eliminated | | | |
| | | | (N-teminal tag #5) | | | |
| 34 | 33TX3 | I18A; F22A; | Epitopes 1 and 2; | 0.473 | 8.8 | 40 |
| | | Q217D; V223T | Epitope 3 eliminated | | | |
| | | | (N-teminal tag #6) | | | |
| 35 | 491TEMX | I18A; F22A; | Epitopes 1 and 2; | 0.188 | 2.9 | 2 |
| | | Q217D; V223T | Epitope 3 not present | | | |

It was found that a specific 11 amino acid C-terminal deletion of Epitope 3 effectively eliminated inflammasome activation without significantly compromising flagellin variant activity (e.g., NF-κB signaling). This discovery allowed for production of a flagellin variant that does not induce IL-1β and IL-18 and thus has less inflammatory toxicity, which leads to better therapeutic activity (e.g., combination with checkpoint inhibitors).

### Example 2: In Vitro Characterization of Improved De-Immunized Flagellin Variants Relative to CBLB502 and 33MX

Epitope mapping data obtained as described above (See Example 1) provided a foundation for the ultimate design of the de-immunized SE-1 and SE-2 lead candidates. These variants, 33TX2 (a/k/a SE-1) and 491TEMX (a/k/a SE-2 and GP532), were characterized in vitro, as compared to entolimod (a/k/a CBLB502).

### Immunogenicity

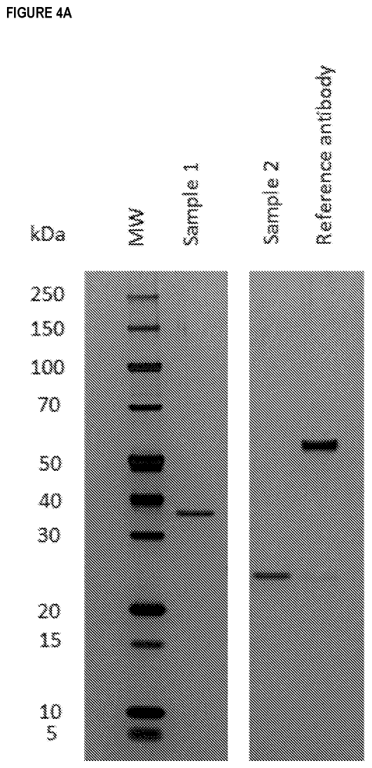

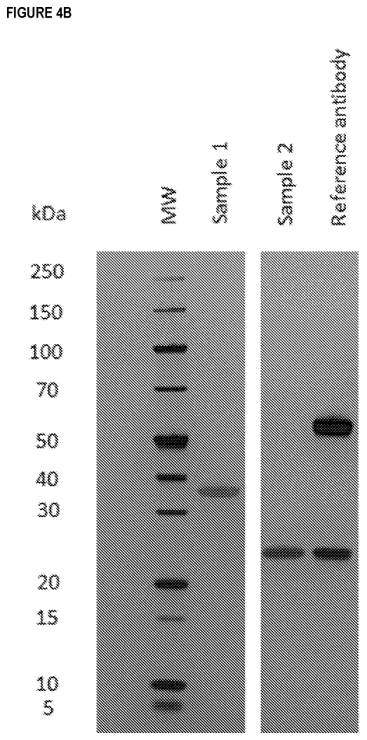

A dendritic cell (DC): T cell assay (EpiScreen™ DC:T cell assay) was used to assess the immunogenic potential of 491TEMX, as compared to CBLB502, by measuring CD4⁺ T cell responses. To assess the immunogenic potential of each sample, the EpiScreen™ DC:T cell assay used two markers (IL-2 production and proliferation) to measure T cell activation. Samples (Sample 1/entolimod and Sample 2/SE-2), as detailed in Table 2 below, were stored according to the instructions provided. The purity of the samples was assessed by denaturing SDS PAGE on a 4-12% gradient gel and silver stained (Pierce Silver Stain Kit, ThermoFisher Scientific, Loughborough, UK). The results of this analysis are shown in FIG. 4A-B, and indicate that there is one band present in both samples (a reference antibody is shown for comparison). Endotoxin levels were measured using a chromogenic kinetic LAL assay kit according to the manufacturer's instructions (Charles River, Margate, UK) and found to be within the limit acceptable for the assay (<5.0 EU/mg) (Table 2). The samples were diluted to 500 μg/ml in AIM-V® culture medium (ThermoFisher Scientific) just before use (final assay concentration 50 μg/ml). KLH was stored at −20° C. at 10 mg/ml in dH2O. For the studies, an aliquot of KLH was thawed immediately before diluting to 1 mg/ml in AIM-V® (final assay concentration 100 μg/ml). PHA (Sigma) was used as a positive control in the ELISpot assay and a 1 mg/ml stock was stored at −20° C. before diluting to a concentration of 10 μg/ml in AIM-V® (final assay concentration 2.5 μg/ml).

TABLE 2
Details of flagellin samples for EpiScreen ™ DC:T
cell immunogenicity analysis.

| | Reference | Concentration | | Endotoxin |
|---|---|---|---|---|
| Sample | ID | (mg/ml) | Storage | (EU/mg) |
| Sample 1/entolimod | CBLB502 | 1.6 | −80° C. | 0.63 |
| Sample 2/SE-2 | 491TEMX | 1.0 | −80° C. | 4.62 |

Monocyte-derived dendritic cells (MoDC) were prepared to a semi-mature stage and incubated with the samples before full DC maturation was induced by stimulation with the pro-inflammatory cytokine TNF-α. Autologous CD4+ T cells were also prepared for proliferation assays. Specifically, MoDC were prepared by reviving PBMC from donors in AIM-V® culture medium and isolating CD14⁺ cells (monocytes) using Miltenyi Pan Monocyte Isolation kits and LS columns (Miltenyi Biotech, Oxford, UK) according to the manufacturer's instructions. Monocytes were resuspended in DC culture media (AIM-V® supplemented with 1000 IU/ml IL-4 and 1000 IU/ml GM-CSF (Peprotech, London, UK)) and plated in low-bind 24 well plates (2 ml final culture volume). Cells were fed on day 2 by half volume DC culture media change. On day 3, antigens (samples and KLH) were added to the cells in DC culture medium to a final concentration of 0.3 μM. The neoantigen KLH was included as a control. In addition, an equivalent volume of DC culture medium was added to the untreated control wells. MoDC were incubated with antigen for 24 hours, after which the cells were washed three times, and resuspended in DC culture medium containing 50 ng/ml TNF-α (Peprotech) in order to mature the cells. Cells were fed again on day 7 by a half volume medium change with DC culture medium containing 50 ng/ml TNF-α before harvesting on day 8. The harvested MoDC were counted and viability assessed using trypan blue (Sigma) dye exclusion. MoDC were then γ-irradiated (40 Gy) before use in the proliferation and ELISpot assays. Also on day 8, autologous CD4⁺ T cells were isolated by negative selection from PBMC using a CD4⁺ T Cell Isolation Kit and LS columns (Miltenyi Biotech) according to the manufacturer's instructions.

Proliferation assays were then performed. After counting and assessing cell viability, 1×10⁵ CD4⁺ T cells were co-cultured with 1×10⁴ irradiated MoDC in 96 well round bottom plates. All cultures were set up in six replicate wells. Following a 7-day co-culture, the cells were pulsed with 1.0 μCi [³H]-Thymidine (Perkin Elmer, Buckinghamshire, UK) in 50 μl AIM-V® medium and incubated for a further 6 hours before harvesting onto filter mats using a TomTec Mach III cell harvester. Cpm for each well were determined by Meltilex™ (Perkin Elmer) scintillation counting on a Microplate Beta Counter in paralux, low background counting.

ELISpot assays were also performed. ELISpot plates (Millipore, Watford, UK) were pre-wetted and coated overnight with 100 μl/well IL-2 capture antibody (R&D Systems, Abingdon, UK) in PBS. Plates were then washed 2 times in PBS, incubated overnight in blocking buffer (1% BSA in PBS) and washed in AIM-V® medium. CD4⁺ T cells and DC were added to each well as for the proliferation assay (ratio 10:1). Each sample was tested in sextuplet cultures and, for each donor, a negative control (AIM-V® medium alone), no cells control and a mitogen positive control (PHA at 2.5 μg/ml—used as an internal test for ELISpot function and cell viability, Sigma) were also included on each plate. After a 7-day incubation period, ELISpot plates were developed by sequential washing in dH2O and PBS (×3) prior to the addition of 100 μl filtered, biotinylated detection antibody (R&D Systems) in PBS/1% BSA. Following incubation at 37° C. for 1.5 hours, plates were further washed in PBS (×3) and 100 μl filtered streptavidin-AP (R&D Systems) in PBS/1% BSA was added for 1.5 hours (incubation at room temperature). Streptavidin-AP was discarded and plates were washed in PBS (×4). 100 μl BCIP/NBT substrate (R&D Systems) was added to each well and incubated for 30 minutes at room temperature. Spot development was stopped by washing the wells and the backs of the wells three times with dH2O. Dried plates were scanned on an Immunoscan® Analyser and spots per well (spw) were determined using Immunoscan® Version 5 software.

Cell viability was assessed following MoDC harvest on day 8, using trypan blue dye exclusion and expressed as a percentage of cells unstained with trypan blue out of the total number of cells. It was found that the samples did not affect cell viability since the mean viability of MoDC treated with medium alone was similar to that of MoDC treated with samples or control antigen (KLH): between 94% and 96%.

For proliferation and IL-2 ELISpot assays, an empirical threshold of an SI equal to or greater than ≥1.90 (SI 1.90) was established whereby samples inducing responses above this threshold were deemed positive. For both proliferation (n=6) and IL-2 ELISpot (n=6) data sets, positive responses were defined by statistical and empirical thresholds: (1) Significance (p<0.05) of the response by comparing cpm or spw of test wells against medium control wells (cpm>150, spw>3) using an unpaired two sample Student's t-test; and (2) SI≥1.90, where SI=mean of test wells (cpm or spw)/baseline (cpm or spw). Data presented in this way is indicated as SI≥1.90, p<0.05. In addition, intra-assay variation was assessed using Dixons Q test in combination with the CV and SD of the raw data from replicate cultures. P values were calculated using an unpaired two sample Student's t-test in Prism 5 (GraphPad, La Jolla, USA).

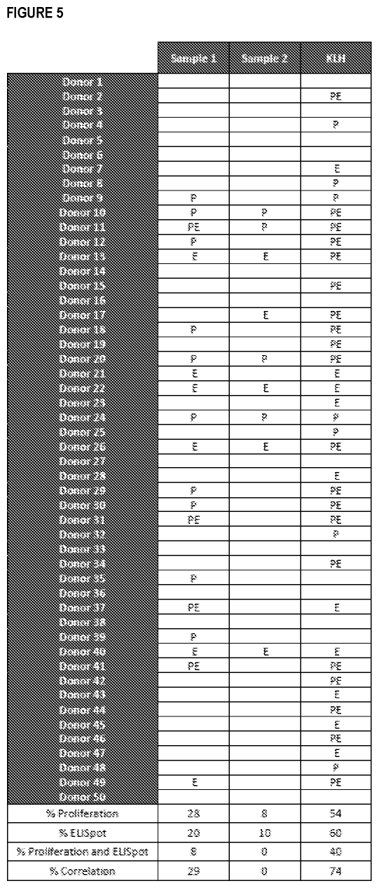

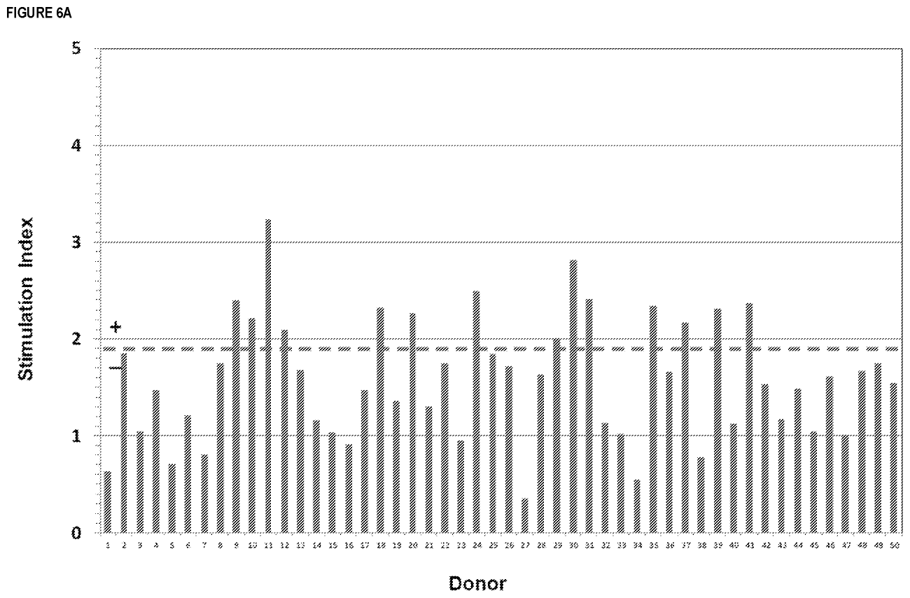

FIG. 5 shows a summary of the CD4+ T cell proliferation in response to the samples. The neo-antigen KLH induced positive responses in 54% of the donor cohort, with a mean magnitude SI of 4.07 (See Table 3). Sample 1/entolimod induced positive responses in 28% of the donor cohort (SI≥1.90 (p<0.05)), whereas Sample 2/SE-2 induced positive responses in 8% of the donor cohort. The mean magnitude of the positive T cell proliferation responses was low (SI<3.00) for both samples with mean SIs of 2.39 and 2.67 for Sample 1/entolimod and Sample 2/SE-2 respectively (Table 3).

TABLE 3
Summary of the mean magnitude (±SD) of positive CD4⁺
T cell proliferative responses against the samples.

| | Reference | Mean | | % |
|---|---|---|---|---|
| Sample | ID | SI | SD | Response |
| Sample 1/entolimod | CBLB502 | 2.39 | ±0.31 | 28 |
| Sample 2/SE-2 | 491TEMX | 2.67 | ±0.98 | 8 |
| KLH | Control | 4.07 | ±2.38 | 54 |

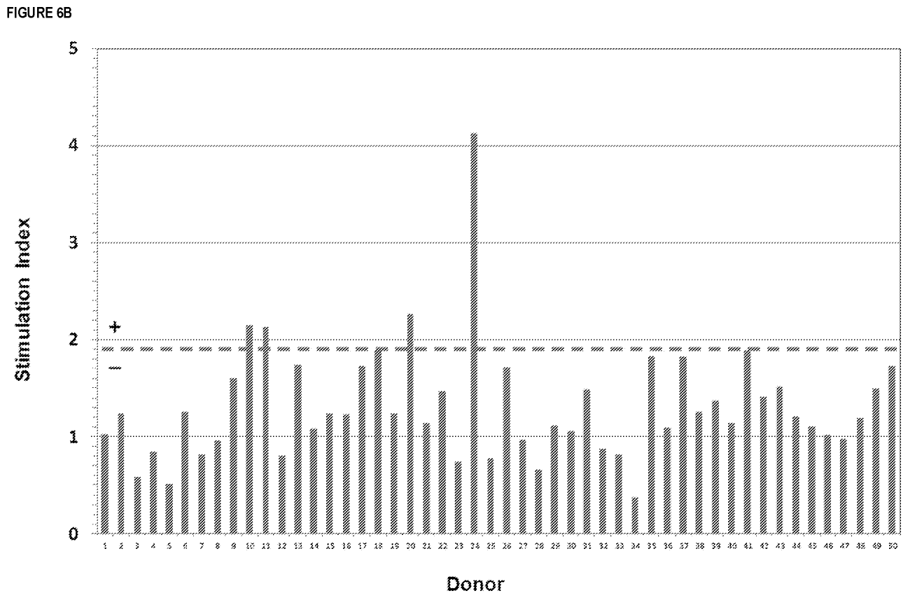

FIG. 6A-C shows healthy donor T cell proliferation responses to: (FIG. 6A) sample 1/entolimod, (FIG. 6B) sample 2/SE-2 and (FIG. 6C) KLH (control). T cell responses with an SI≥1.90 (indicated by red dotted line) that were significant (p<0.05) using an unpaired, two sample Student's t-test were considered positive.

Table 4 shows a summary of the responses obtained in the IL-2 ELISpot assay, which measures IL-2 secretion by CD4⁺ T cells following stimulation with DC loaded with the two samples and KLH. Similar to the proliferation assay, positive responses were recorded in donors that produced an SI 1.90 with a significant (p<0.05) difference observed between test spw and background (untreated medium control). All positive control PHA treated wells were positive for the presence of spots, although SI values are not prepared for the ELISpot data as after 7 days the majority of wells contained spots too numerous to count (data not shown). KLH induced a positive response in 60% of donors with a mean magnitude SI of 3.82. The results obtained in the IL-2 ELISpot assay for the samples were similar to those obtained in the proliferation assay with sample 1 inducing a higher response rate. Samples 1/entolimod and 2/SE-2 induced an IL-2 response frequency of 20% and 10% respectively and these were all significant (p<0.05) using an unpaired, two sample Student's t-test (See Table 4). The mean magnitude (SI) of the positive T cell responses in the IL-2 ELISpot assay for sample 1/entolimod was 2.92 and 3.12 for sample 2/SE-2 (Table 4).

TABLE 4
Summary of the frequency and magnitude (±SD) of positive
IL-2 secretion responses against the two samples and KLH.

| | Reference | Mean | | % |
|---|---|---|---|---|
| Sample | ID | SI | SD | Response |
| Sample 1/entolimod | CBLB502 | 2.92 | ±1.44 | 20 |
| Sample 2/SE-2 | 491TEMX | 3.12 | ±2.02 | 10 |
| KLH | Control | 3.82 | ±3.07 | 60 |

FIG. 7A-C depicts healthy donor T cell IL-2 secretion response to: (FIG. 7A) sample 1, (FIG. 7B) sample 2 and (FIG. 7C) KLH. CD4⁺ T cells were incubated with autologous mature DC loaded with the samples and assessed for IL-2 secretion after 7 days' incubation. T cell responses with an SI≥1.90 (indicated by red dotted line) that were significant (p<0.05) using an unpaired, two sample Student's t-test were considered positive.

The results showed that both samples induced a combined positive response frequency in 0-8% of the donor cohort. However, the correlation between positive proliferation and IL-2 ELISpot responses for the test samples was low. In the individual assays, sample 1/entolimod would be considered of greater risk of clinical immunogenicity than sample 2/SE-2 due to the high frequency of positive proliferation (28% vs 8%) and IL-2 ELISpot (20% vs 10%) responses. In addition, the mean magnitude of proliferative responses to sample 1/entolimod was significantly higher than those to sample 2/SE-2, adding further evidence to support the conclusion of an increased risk of clinical immunogenicity for sample 1/entolimod versus sample 2/SE-2.

### NF-κB Activity

FIG. 8 depicts analysis of flagellin variants tested for ability to induce NF-κB signaling in 293-hTLR5-LacZ reporter cells, where the cells were incubated in the presence of test agents (e.g., flagellin variants). It was found that there was less than a 5-fold reduction in NF-κB activity induced by flagellin variants 33TX2 and 491TEMX, as compared to activity induced by the 33MX variant. Indeed, Table 5 depicts a summary of the activity potency exhibited by flagellin variants as compared to CBLB502 and as compared to the variant 33MX. EC50 is defined as the concentration of an agent which induces a response halfway between baseline and maximum after a specified exposure time. For example, EC50 values calculated from the reporter enzyme activity dose-response curves (Table 5) show that 491TEMX exhibited less than 5-fold reduction in NF-κB activity compared to 33MX and entolimod/CBLB502, respectively (EC50=0.114 ng/ml compared to 0.043 and 0.032 ng/ml).

TABLE 5
Summary of activity exhibited by flagellin
variants in terms of potency.

| | EC50, | | EC50, | Mut. EC50/ | Mut. EC50/ |
|---|---|---|---|---|---|
| | ng/ml | MW | pM | EC50 33MX | EC50 CBLB502 |
| 33MX | 0.043 | 26190 | 1.634 | | 1.78 |
| 33TX2 | 0.137 | 25026 | 5.465 | 3.34 | 5.94 |
| 491TEMX | 0.114 | 26200 | 4.353 | 2.66 | 4.73 |
| CBLB502 | 0.032 | 34984 | 0.920 | | |

### Inflammasome Activation

The NLRC4 inflammasome is one of a number of cytoplasmic multi-molecule complexes that is assembled following activation of its pattern recognition receptor (PRR) component by a microbial entity. In the case of NLRC4, the cytoplasmic Nod-like receptor (NLR) is activated by bacterial flagellin, which is also an agonist of Toll-like receptor 5 (TLR5) on the cell membrane. It is assumed that extracellular flagellin is able to activate the cytoplasmic NLRC4 inflammasome due to internalization of flagellin-TLR5 complexes. Once assembled, the inflammasome initiates a pro-inflammatory cascade involving caspase-1 activation and, subsequently, processing of pro-IL-1β to mature IL-1β, which is major pro-inflammatory cytokine. Therefore, measurement of IL-1β production provides a readout of inflammasome activity.

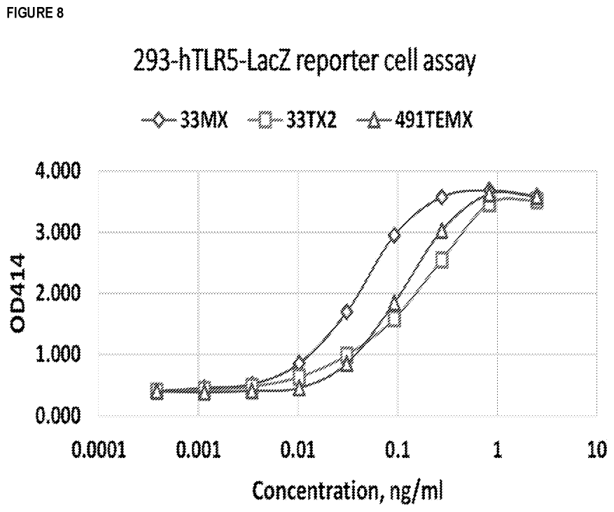

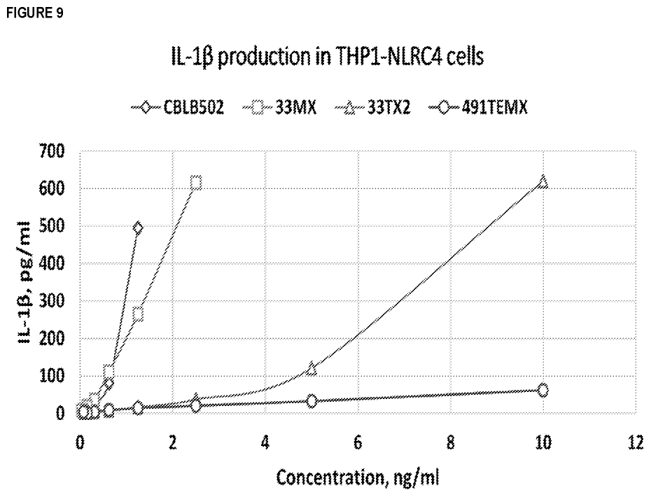

Flagellin variants were tested in THP1-NLRC4 cells in order to determine inflammasome activation (e.g., IL-1β and IL-18 production as markers) as compared to CBLB502 and other flagellin variants. Structure-activity relationship (SAR) of flagellin variants was assessed by using two in vitro cell-based readouts: (i) inflammasome activation, and (ii) NF-κB signaling. The THP1-NLRC4 cells were cultured in the presence of the test agents (e.g., flagellin variants). NF-κB signaling induced by variants 33TX2 and 491TEMX in reporter cells was shown previously in FIG. 8, where it is shown that the NF-κB activity induced by variants 33TX2 and 491TEMX is retained and comparable to that induced by the 33MX variant. FIG. 9 shows low—almost no—inflammasome activity (e.g., IL-1β production) induced by the variant 491TEMX in THP1-NLRC4 cells, as compared to inflammasome activity induced by CBLB502 and variants 33MX and 33TX2 in THP1-NLRC4 cells. The results show that the variant 491TEMX induces minimal inflammasome activation as compared to CBLB502, 33MX, and 33TX2 at increasing concentrations.

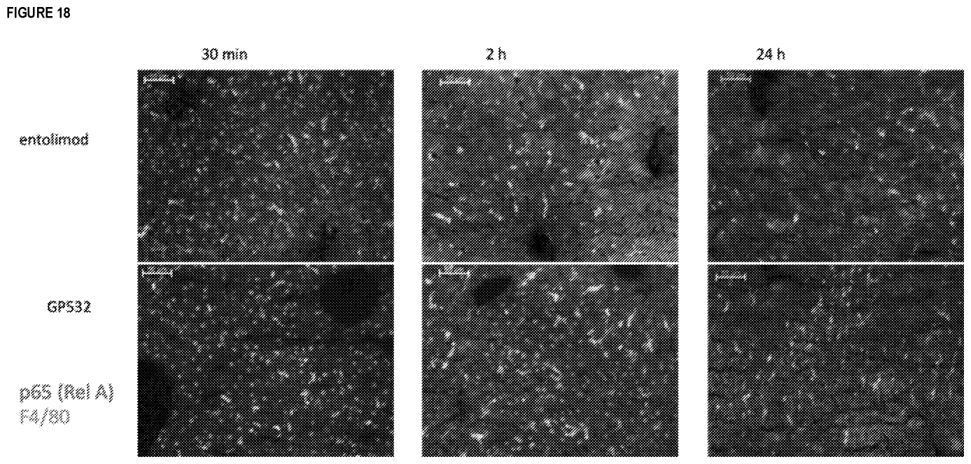

Further, FIG. 18 depicts histological analysis of mouse liver hepatocytes and shows NF-κB activation by GP532 (a/k/a 491TEMX) and entolimod. Both entolimod and GP532 treatments resulted in robust nuclear translocation of p65 at 30 min after injection. In entolimod-treated mice amount of nuclear p65 decreased significantly 2 h post-injection and it was completely absent at 24 h. In mice treated with GP532 (but not entolimod) strong nuclear staining persisted at 2 h post-injection. No staining was observed at 24 h after injection of either drug. These results show that GP532 provides improved dynamics (e.g., longer duration) of NF-κB signaling response in liver cells.

### Example 3. In Vivo Characterization of Improved De-Immunized Flagellin Variants Relative to CBLB502 and 33MX

In vivo testing of the signaling activity of the flagellin variants appeared to be consistent with the in vitro data, in that the variant 491TEMX (i.e., SE-2) performed as good as or better than entolimod. A pharmacokinetics profile was established by injection of 1 μg of flagellin variants, SE-1 and SE-2, and entolimod into reporter mice and measurement of the resultant concentration in ng/ml over the course of 24 hours (FIG. 10). The results shown in FIG. 10 conclude that SE-2 performed better or equal to entolimod.

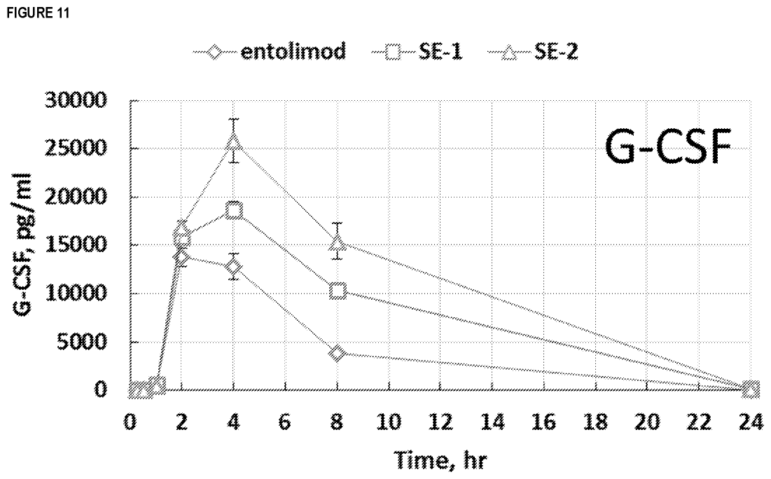

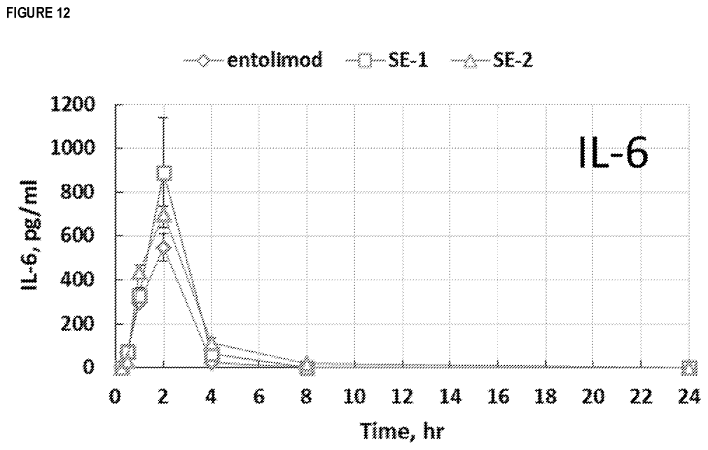

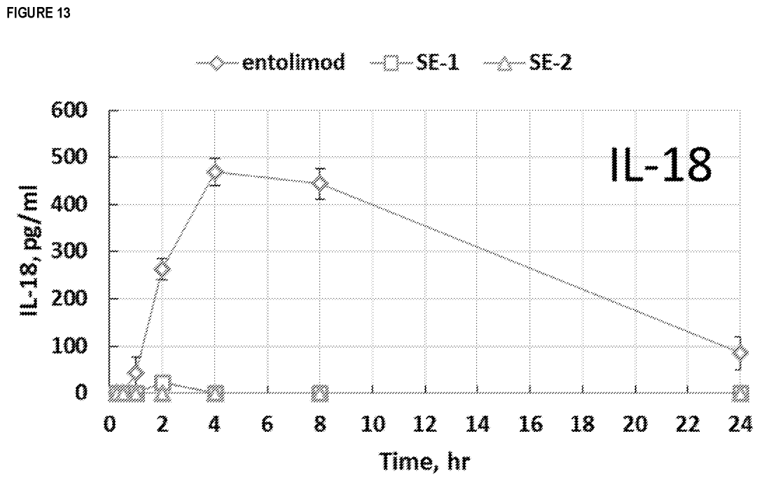

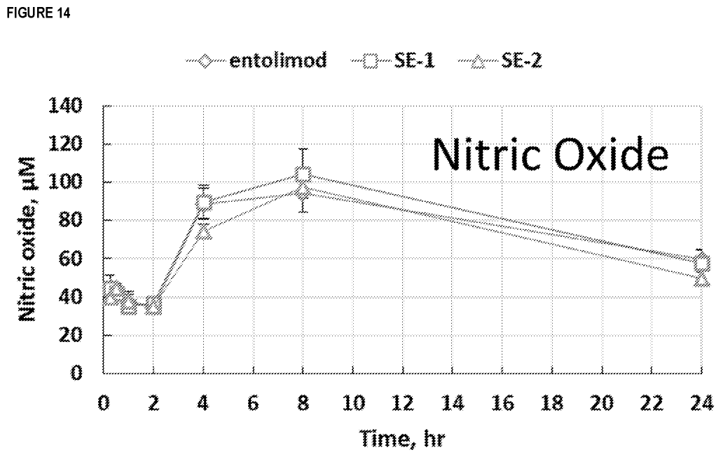

Additionally, various other markers were measured over the course of 24 hours in mice that were injected with 1 μg of flagellin variants, SE-1 and SE-2, and entolimod. For example, pharmacodynamics markers, including cytokines G-CSF (FIG. 11) and IL-6 (FIG. 12), were measured over the course of 24 hours. The inflammasome marker IL-18 was measured, as shown in FIG. 13, with the results indicating a confirmation of the in vitro results, that is, that presentation of the flagellin variants SE-1 (a/k/a 33TX2) and SE-2 (a/k/a 491TEMX) induced substantially lower inflammasome activation as compared to entolimod (a/k/a, CBLB502). Nitric oxide production was also measured, and the results in FIG. 14 show that over time, the levels of nitric oxide induced by variants SE-1 (a/k/a 33TX2) and SE-2 (a/k/a 491TEMX) were similar to levels of nitric oxide induced by entolimod.

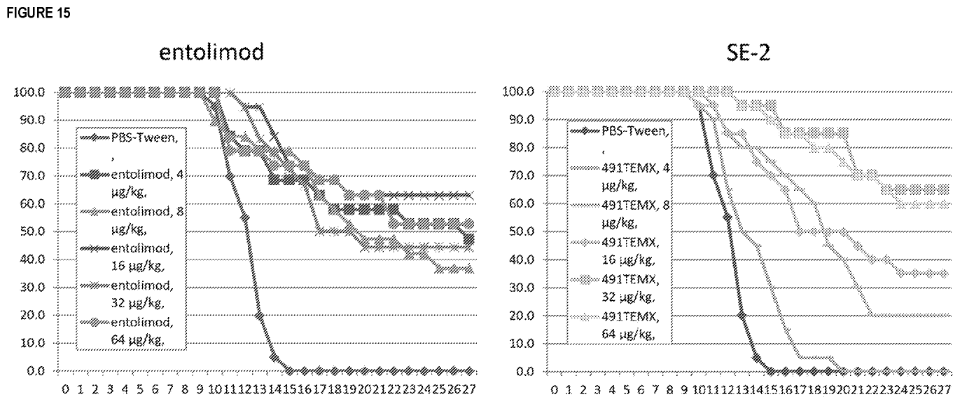

In vivo characterization of radioprotective activity was analyzed by an experiment that involved injection of various doses of entolimod and SE-2 (a/k/a 491TEMX) in mice 20 minutes before total body irradiation, followed by monitoring for 27 days. The doses of entolimod and SE-2 were 4 μg/kg, 6 μg/kg, 8 μg/kg, 16 μg/kg, 32 μg/kg, and 64 μg/kg for each. PBS-Tween was used as a control. The results depicted in FIG. 15 show that neither entolimod nor SE-2 (a/k/a 491TEMX) reached 100% radioprotective activity; however, SE-2 (a/k/a 491TEMX) did not perform worse than entolimod. Indeed, the results of FIG. 15 show that the most effective doses for each entolimod and SE-2 are different (16 μg/kg for entolimod and 32 μg/kg for SE-2).

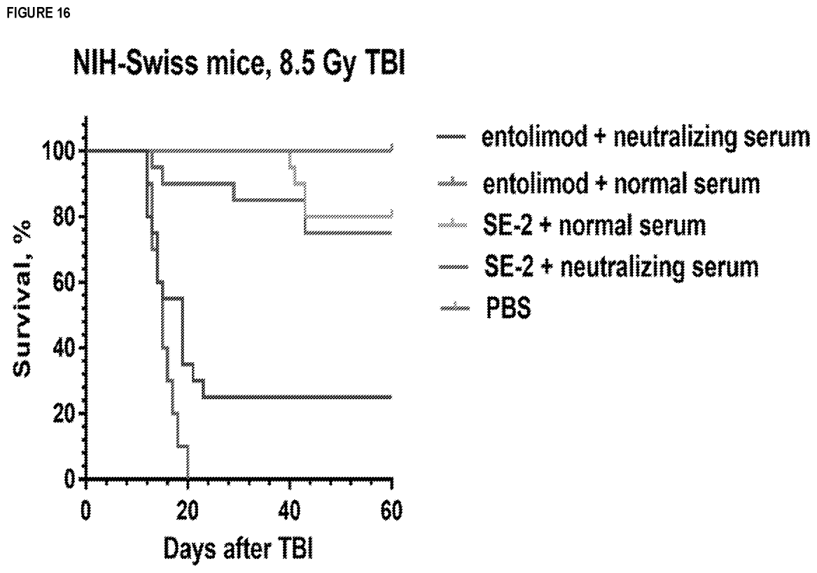

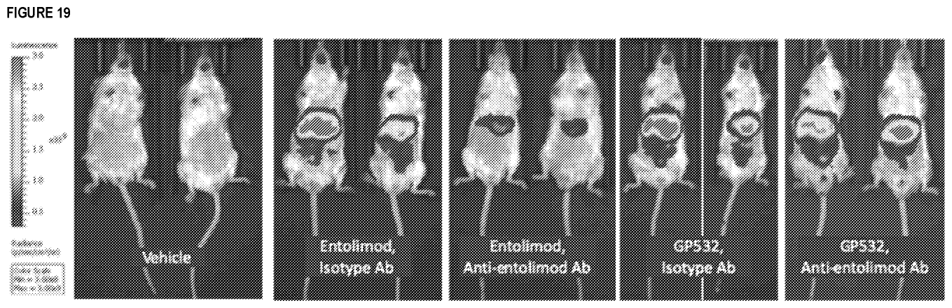

Radioprotective activity was further assessed via a passive serum transfer experiment. Human serum (both normal serum and serum containing neutralizing antibodies) was transferred into NIH-Swiss mice, followed by injection of either entolimod, SE-2 or PBS. The mice were then subjected to total body irradiation (8.5 Gy TBI) and survival was monitored for 60 days after. The results, depicted in FIG. 16, show that neutralizing B cell epitopes affect the efficacy of entolimod treatment but not affect the efficacy of SE-2 (a/k/a 491TEMX) treatment. FIG. 19 depicts in vivo imaging of signaling activity in NF-κB-luciferase reporter mice upon transfusion of neutralizing or non-neutralizing (control) human serum followed by subcutaneous injection of Entolimod or GP532. The results show that administration of GP532 provides resistance to neutralizing antibodies in reporter mice transfused with neutralizing antibodies.

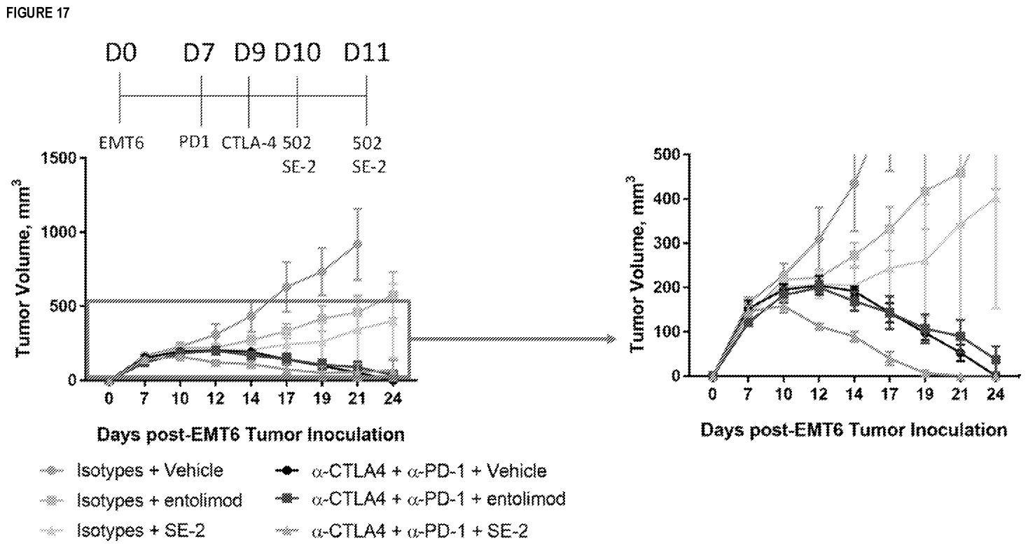

A study was conducted on the effects of a combination tumor treatment with SE-2 (a/k/a GP532 and 491TEMX) and checkpoint inhibitors, in order to establish beneficial properties. An EMT6 mouse model of triple-negative breast cancer was used, where treatment began with administration of checkpoint inhibitors, followed by administration of entolimod or SE-2. Specifically, the mice were given a dose of α-PD1 on day 7, followed by a dose of α-CTLA4 on day 9. On days 10 and 11, doses of entolimod and SE-2 were administered. The results depicted in FIG. 17 show that the combination of administration of checkpoint inhibitors followed by administration of SE-2 exhibited faster tumor regression than administration of entolimod with checkpoint inhibitors or checkpoint inhibitors alone.

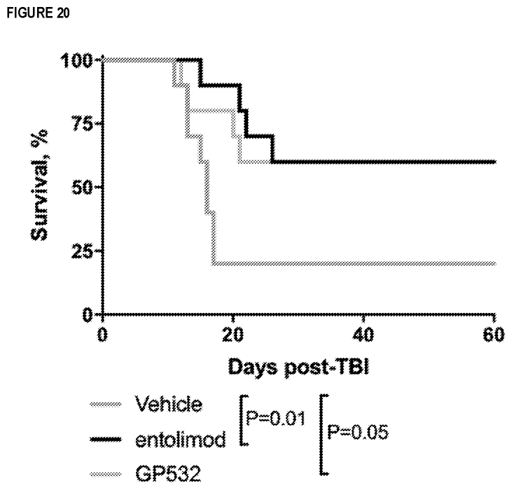

Radiomitigation activity was assessed in BALB/c mice that received injection of vehicle (PBS), entolimod, or GP532. The mice were then subjected to total body irradiation (8.5 Gy TBI) and survival was monitored for 60 days after. The results, depicted in FIG. 20, show that GP532 provided about the same level of radiomitigation activity as entolimod.

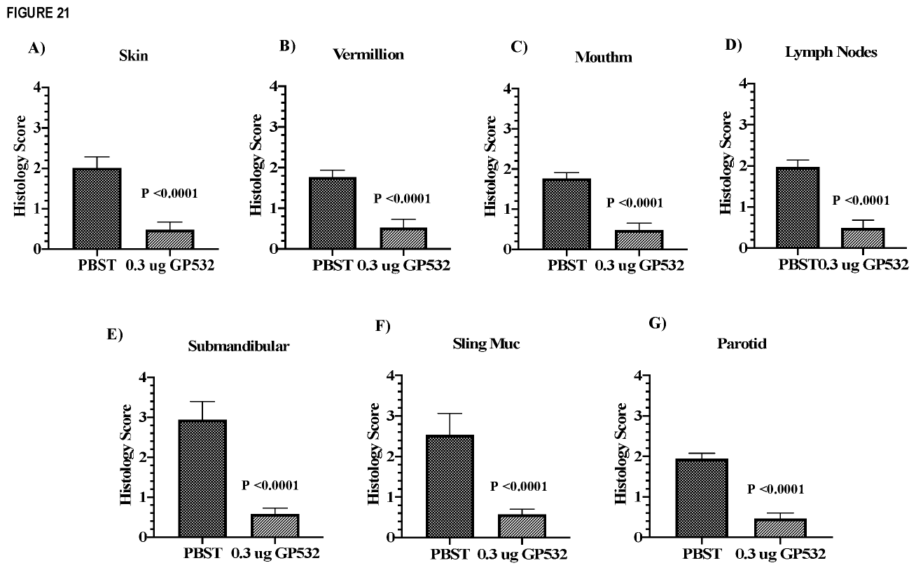

In an additional study, a head and neck cancer mouse model, subjected to localized radiation, received injection of either PBST or 0.3 μg GP532. Various areas of the mice (e.g., skin, vermillion, mouth, lymph nodes, submandibular, sling mucosa, and parotid) were subjected to histology scoring in order to assess the mitigation of localized radiation side effects. The results are depicted in FIG. 21A-G for skin (FIG. 21A), vermillion (FIG. 21B), mouth (FIG. 21C), lymph nodes (FIG. 21D), submandibular (FIG. 21E), sling mucosa (FIG. 21F), and parotid (FIG. 21G), showing that histology scores for each area of the mouth were reduced in the mice that received a 0.3 μg GP532 injection.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A flagellin variant comprising an amino acid sequence having at least 90% sequence identity with SEQ ID NO: 1 and (i) a substitution mutation at a position corresponding to one or more of I18, F22, T23, S24, and K27, and (ii) a substitution mutation at a position corresponding to one or more of I215, L216, Q217, T221, and V223,
wherein the substituted amino acid residue is any naturally-occurring amino acid, and
wherein the flagellin variant retains NF-kB signaling activity.

2. The flagellin variant of claim 1, wherein the substituted amino acid residue is a hydrophilic or hydrophobic amino acid residue.

3. The flagellin variant of any one of the above claims, wherein the hydrophilic amino acid residue is a polar and neutral of charge hydrophilic residue, selected from asparagine (N), glutamine (Q), serine (S), threonine (T), proline (P), and cysteine (C).

4. The flagellin variant of any one of the above claims, wherein the hydrophilic amino acid residue is a polar and negatively charged hydrophilic residue, selected from aspartate (D) and glutamate (E).

5. The flagellin variant of any one of the above claims, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

6. A flagellin variant comprising an amino acid sequence having at least 90% sequence identity with SEQ ID NO: 1 and (i) a substitution mutation selected from:
a hydrophobic residue other than isoleucine (I) at a position corresponding to 18,
a hydrophobic residue other than phenylalanine (F) at a position corresponding to 22,
a hydrophilic residue other than threonine (T) at a position corresponding to 23,
a hydrophilic residue other than serine (S) at a position corresponding to 24, and
a hydrophilic residue other than lysine (K) at a position corresponding to 27;
and (ii) a substitution mutation selected from:
a hydrophobic residue other than isoleucine (I) at a position corresponding to 215,
a hydrophobic residue other than leucine (L) at a position corresponding to 216,
a hydrophilic residue other than glutamine (Q) at a position corresponding to 217,
a hydrophilic residue other than threonine (T) at a position corresponding to 221, and
a hydrophilic residue other than valine (V) at a position corresponding to 223,
wherein the flagellin variant retains NF-kB signaling activity.

7. The flagellin variant of any one of the above claims, wherein the hydrophilic amino acid residue is a polar and neutral of charge hydrophilic residue, selected from asparagine (N), glutamine (Q), serine (S), threonine (T), proline (P), and cysteine (C).

8. The flagellin variant of any one of the above claims, wherein the hydrophilic amino acid residue is a polar and negatively charged hydrophilic residue, selected from aspartate (D) and glutamate (E).

9. The flagellin variant of any one of the above claims, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

10. A flagellin variant comprising amino acid sequence having at least 90% sequence identity with SEQ ID NO: 1 and (i) a substitution mutation at a position corresponding to one or more of 118 and F22, and (ii) a substitution mutation at a position corresponding to one or more of Q217 and V223, wherein the substituted amino acid residue is any naturally-occurring amino acid, and wherein the flagellin variant retains NF-kB signaling activity.

11. The flagellin variant of claim 1, wherein the substituted amino acid residue is a hydrophilic or hydrophobic amino acid residue.

12. The flagellin variant of any one of the above claims, wherein the hydrophilic amino acid residue is a polar and neutral of charge hydrophilic residue, selected from asparagine (N), glutamine (Q), serine (S), threonine (T), proline (P), and cysteine (C).

13. The flagellin variant of any one of the above claims, wherein the hydrophilic amino acid residue is a polar and negatively charged hydrophilic residue, selected from aspartate (D) and glutamate (E).

14. The flagellin variant of any one of the above claims, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

15. A flagellin variant comprising an amino acid sequence having at least 90% sequence identity with SEQ ID NO: 1 and (i) a substitution mutation selected from:
a hydrophobic residue other than isoleucine (I) at a position corresponding to 18 and a hydrophobic residue other than phenylalanine (F) at a position corresponding to 22;
and (ii) a substitution mutation selected from:
a hydrophilic residue other than glutamine (Q) at a position corresponding to 217 and a hydrophilic residue other than valine (V) at a position corresponding to 223, wherein the flagellin variant retains NF-kB signaling activity.

16. The flagellin variant of claim 15, comprising an amino acid sequence having at least 90% sequence identity with SEQ ID NO: 1 and the following substitution mutations:
a hydrophobic residue other than isoleucine (I) at a position corresponding to 18;
and a hydrophobic residue other than phenylalanine (F) at a position corresponding to 22;
a hydrophilic residue other than glutamine (Q) at a position corresponding to 217; and
a hydrophilic residue other than valine (V) at a position corresponding to 223,

17. The flagellin variant of any one of claims 15-16, wherein the hydrophilic amino acid residue is a polar and neutral of charge hydrophilic residue, selected from asparagine (N), glutamine (Q), serine (S), threonine (T), proline (P), and cysteine (C).

18. The flagellin variant of any one of claims 15-17, wherein the hydrophilic amino acid residue is a polar and negatively charged hydrophilic residue, selected from aspartate (D) and glutamate (E).

19. The flagellin variant of any one of claims 15-18, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

20. The flagellin variant of any one of claims 15-19, wherein the hydrophobic residue other than isoleucine (I) at position corresponding to 18 is alanine (A).

21. The flagellin variant of any one of claims 15-20, wherein the hydrophobic residue other than phenylalanine (F) at position corresponding to 22 is alanine (A).

22. The flagellin variant of any one of claims 15-21, wherein the hydrophilic residue other than glutamine (Q) at position corresponding to 217 is aspartate (D).

23. The flagellin variant of any one of claims 15-22, wherein the hydrophilic residue other than valine (V) at position corresponding to 223 is threonine (T).

24. The flagellin variant of any one of claims 15-23, comprising 118A, F22A, Q217D, and V223T.

25. A flagellin variant comprising an amino acid sequence having at least 90% sequence identity with SEQ ID NO: 6 and (i) a substitution mutation at a position corresponding to one or more of I18, F22, T23, S24, and K27, and (ii) a substitution mutation at a position corresponding to one or more of I215, L216, Q217, T221, and V223,
wherein the substituted amino acid residue is any naturally-occurring amino acid, and
wherein the flagellin variant retains NF-kB signaling activity.

26. The flagellin variant of claim 25, wherein the substituted amino acid residue is a hydrophilic or hydrophobic amino acid residue.

27. The flagellin variant of any one of the above claims, wherein the hydrophilic amino acid residue is a polar and neutral of charge hydrophilic residue, selected from asparagine (N), glutamine (Q), serine (S), threonine (T), proline (P), and cysteine (C).

28. The flagellin variant of any one of the above claims, wherein the hydrophilic amino acid residue is a polar and negatively charged hydrophilic residue, selected from aspartate (D) and glutamate (E).

29. The flagellin variant of any one of the above claims, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

30. A flagellin variant comprising an amino acid sequence having at least 90% sequence identity with SEQ ID NO: 6 and (i) a substitution mutation selected from:
a hydrophobic residue other than isoleucine (I) at a position corresponding to 18,
a hydrophobic residue other than phenylalanine (F) at a position corresponding to 22,
a hydrophilic residue other than threonine (T) at a position corresponding to 23,
a hydrophilic residue other than serine (S) at a position corresponding to 24, and
a hydrophilic residue other than lysine (K) at a position corresponding to 27;
and (ii) a substitution mutation selected from:
a hydrophobic residue other than isoleucine (I) at a position corresponding to 215,
a hydrophobic residue other than leucine (L) at a position corresponding to 216,
a hydrophilic residue other than glutamine (Q) at a position corresponding to 217,
a hydrophilic residue other than threonine (T) at a position corresponding to 221, and
a hydrophilic residue other than valine (V) at a position corresponding to 223,
wherein the flagellin variant retains NF-kB signaling activity.

31. The flagellin variant of any one of the above claims, wherein the hydrophilic amino acid residue is a polar and neutral of charge hydrophilic residue, selected from asparagine (N), glutamine (Q), serine (S), threonine (T), proline (P), and cysteine (C).

32. The flagellin variant of any one of the above claims, wherein the hydrophilic amino acid residue is a polar and negatively charged hydrophilic residue, selected from aspartate (D) and glutamate (E).

33. The flagellin variant of any one of the above claims, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

34. A flagellin variant comprising an amino acid sequence having at least 90% sequence identity with SEQ ID NO: 6 and (i) a substitution mutation at a position corresponding to one or more of I18 and F22, and (ii) a substitution mutation at a position corresponding to one or more of Q217 and V223, wherein the substituted amino acid residue is any naturally-occurring amino acid, and wherein the flagellin variant retains NF-kB signaling activity.

35. The flagellin variant of claim 25, wherein the substituted amino acid residue is a hydrophilic or hydrophobic amino acid residue.

36. The flagellin variant of any one of the above claims, wherein the hydrophilic amino acid residue is a polar and neutral of charge hydrophilic residue, selected from asparagine (N), glutamine (Q), serine (S), threonine (T), proline (P), and cysteine (C).

37. The flagellin variant of any one of the above claims, wherein the hydrophilic amino acid residue is a polar and negatively charged hydrophilic residue, selected from aspartate (D) and glutamate (E).

38. The flagellin variant of any one of the above claims, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

39. A flagellin variant comprising an amino acid sequence having at least 90% sequence identity with SEQ ID NO: 6 and (i) a substitution mutation selected from:
a hydrophobic residue other than isoleucine (I) at a position corresponding to 18 and a hydrophobic residue other than phenylalanine (F) at a position corresponding to 22;
and (ii) a substitution mutation selected from:
a hydrophilic residue other than glutamine (Q) at a position corresponding to 217 and a hydrophilic residue other than valine (V) at a position corresponding to 223, wherein the flagellin variant retains NF-kB signaling activity.

40. The flagellin variant of claim 39, comprising an amino acid sequence having at least 90% sequence identity with SEQ ID NO: 6 and the following substitution mutations:
a hydrophobic residue other than isoleucine (I) at a position corresponding to 18;
a hydrophobic residue other than phenylalanine (F) at a position corresponding to 22;
a hydrophilic residue other than glutamine (Q) at a position corresponding to 217; and
a hydrophilic residue other than valine (V) at a position corresponding to 223,

41. The flagellin variant of any one of claims 39-40, wherein the hydrophilic amino acid residue is a polar and neutral of charge hydrophilic residue, selected from asparagine (N), glutamine (Q), serine (S), threonine (T), proline (P), and cysteine (C).

42. The flagellin variant of any one of claims 39-41, wherein the hydrophilic amino acid residue is a polar and negatively charged hydrophilic residue, selected from aspartate (D) and glutamate (E).

43. The flagellin variant of any one of claims 39-42, wherein the hydrophobic amino acid residue is a hydrophobic, aliphatic amino acid residue, selected from glycine (G), alanine (A), leucine (L), isoleucine (I), methionine (M), and valine (V), or a hydrophobic, aromatic amino acid residue, selected from phenylalanine (F), tryptophan (W), and tyrosine (Y).

44. The flagellin variant of any one of claims 39-43, wherein the hydrophobic residue other than isoleucine (I) at a position corresponding to 18 is alanine (A).

45. The flagellin variant of any one of claims 39-44, wherein the hydrophobic residue other than phenylalanine (F) at a position corresponding to 22 is alanine (A).

46. The flagellin variant of any one of claims 39-45, wherein the hydrophilic residue other than glutamine (Q) at a position corresponding to 217 is aspartate (D).

47. The flagellin variant of any one of claims 39-46, wherein the hydrophilic residue other than valine (V) at a position corresponding to 223 is threonine (T).

48. The flagellin variant of any one of claims 39-47, comprising 118A, F22A, Q217D, and V223T.

49. A polynucleotide comprising a polynucleotide sequence encoding the flagellin variant of any one of the above claims.

50. A host cell comprising the polynucleotide of claim 49.

51. The flagellin variant of any one of the above claims, characterized in that the flagellin variant exhibits low inflammasome activity.

52. The flagellin variant of any one of the above claims, wherein the flagellin variant exhibits lower inflammasome activity relative to inflammasome activity exhibited by flagellin derivatives having the amino acid sequences of SEQ ID NO: 1, SEQ ID NO: 3 and/or SEQ ID NO: 6.

53. The flagellin variant of any one of the above claims, characterized in that the flagellin variant exhibits reduced T cell immunogenicity.

54. The flagellin variant of any one of the above claims, wherein the flagellin variant exhibits reduced T cell immunogenicity relative to T cell immunogenicity exhibited by flagellin derivatives having the amino acid sequences of SEQ ID NO: 1, SEQ ID NO: 3 and/or SEQ ID NO: 6.

55. The flagellin variant of any one of the above claims, wherein the flagellin variant retains NF-kβ signaling activity.

56. The flagellin variant of any one of the above claims, wherein the flagellin variant exhibits similar or higher NF-kB signaling activity relative to NF-kβ signaling activity exhibited by flagellin derivatives having the amino acid sequences of SEQ ID NO: 1, SEQ ID NO: 3 and/or SEQ ID NO: 6.

57. The flagellin variant of any one of the above claims, wherein the flagellin variant retains radioprotective or radiomitigation activity.

58. The flagellin variant of any one of the above claims, wherein the flagellin variant exhibits similar or better radioprotective or radiomitigation activity relative to radioprotective or radiomitigation activity exhibited by flagellin derivatives having the amino acid sequences of SEQ ID NO: 1, SEQ ID NO: 3 and/or SEQ ID NO: 6.

59. The flagellin variant of any one of the above claims, wherein the flagellin variant demonstrates improved resistance to neutralizing B cell epitopes.

60. The flagellin variant of any one of the above claims, wherein the flagellin variant demonstrates improved resistance to neutralizing B cell epitopes relative to resistance to neutralizing B cell epitopes of flagellin derivatives having the amino acid sequences of SEQ ID NO: 1, SEQ ID NO: 3 and/or SEQ ID NO: 6.

61. The flagellin variant of any one of the above claims, wherein the flagellin variant induces expression of one or more of the cytokines.

62. The flagellin variant of any one of the above claims, wherein the flagellin variant induces expression of one or more of the cytokines selected from G-CSF, IL-6, IL-12, keratinocyte chemoattractant (KC), IL-10, MCP-1, TNF-α, MIG, and MIP-2.

63. A pharmaceutical composition comprising the flagellin variant of any one of the above claims and a pharmaceutically acceptable carrier.

64. A method of stimulating TLR5 signaling comprising administering the flagellin variant of any one of the above claims.

65. The method of claim 64, wherein the subject suffers from cancer.

66. The method of claim 65, wherein the tumor expresses TLR5 or the tumor does not express TLR5.

67. The method of claim 65, wherein the cancer is selected from breast cancer, lung cancer, colon cancer, kidney cancer, liver cancer, ovarian cancer, prostate cancer, testicular cancer, genitourinary tract cancer, lymphatic system cancer, rectal cancer, pancreatic cancer, esophageal cancer, stomach cancer, cervical cancer, thyroid cancer, skin cancer, leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, and Burkett's lymphoma, acute and chronic myelogenous leukemias, myelodysplastic syndrome, myeloid leukemia, promyelocytic leukemia, astrocytoma, neuroblastoma, glioma, schwannomas, fibrosarcoma, rhabdomyoscarcoma, osteosarcoma, xenoderma pigmentosum, keratoactanthoma, seminoma, thyroid follicular cancer, teratocarcinoma, and cancers of the gastrointestinal tract or the abdominopelvic cavity.

68. The method of claim 64, wherein the subject suffers from radiation-induced damage.

69. The method of claim 68, wherein the subject has been subjected to a lethal dose of radiation.

70. The method of claim 68, wherein the subject is undergoing radiation treatment.

71. The method of claim 68, wherein the flagellin variant is administered prior to exposure to radiation.

72. The method of claim 68, wherein the flagellin variant is administered during exposure to radiation.

73. The method of claim 68, wherein the flagellin variant is administered after exposure to radiation.

74. The method of any one of claims 64-73, wherein the flagellin variant is administered in conjunction with other therapeutics and/or treatments.

75. The method of claim 74, the flagellin variant is administered in conjunction with chemotherapy.

76. The method of claim 74, the flagellin variant is administered with radiation treatment.

77. The method of claim 74, wherein the flagellin variant is administered in conjunction with an antioxidant.

78. The method of claim 74, wherein the flagellin variant is administered in conjunction with one or more checkpoint inhibitors.

79. The method of claim 78, wherein the one or more checkpoint inhibitors is selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

80. The method of claim 74, wherein the flagellin variant is administered prior to administration of other therapeutics and/or treatments.

81. The method of claim 74, wherein the flagellin variant is administered at the same time as other therapeutics and/or treatments.

82. The method of claim 74, wherein the flagellin variant is administered after administration of other therapeutics and/or treatments.

83. A method of treating cancer comprising administering the flagellin variant of any one of the above claims to a subject in need thereof.

84. A method of treating radiation-induced damage comprising administering the flagellin variant of any one of the above claims to a subject in need thereof.

85. A method of treating aging or an age-related disorder comprising administering the flagellin variant of any one of the above claims to a subject in need thereof.

86. The method of claim 85, wherein the age-related disorder is selected from Alzheimer's disease, type II diabetes, macular degeneration, chronic inflammation-based pathologies (e.g., arthritis), atherosclerosis, cancer types known to be associated with aging (e.g., prostate cancer, melanoma, lung cancer, colon cancer), Hutchinson-Gilford progeria and Werner's Syndrome.

87. The method of any one of claims 64-82, wherein the flagellin variant comprises an amino acid sequence having about 95%, or about 97%, or about 98%, or about 99% sequence identity to SEQ ID NO: 2.

88. The method of any one of claims 64-82, wherein the flagellin variant comprises the amino acid sequence of SEQ ID NO: 2.

89. A flagellin variant comprising an amino acid sequence having at least 90% sequence identity with SEQ ID NO: 2.

90. A flagellin variant comprising an amino acid sequence having at least 93% sequence identity with SEQ ID NO: 2.

91. A flagellin variant comprising an amino acid sequence having at least 94% sequence identity with SEQ ID NO: 2.

92. A flagellin variant comprising an amino acid sequence having at least 95% sequence identity with SEQ ID NO: 2.

93. A flagellin variant comprising an amino acid sequence having at least 97% sequence identity with SEQ ID NO: 2.

94. A flagellin variant comprising an amino acid sequence having at least 98% sequence identity with SEQ ID NO: 2.

95. A flagellin variant comprising an amino acid sequence having at least 99% sequence identity with SEQ ID NO: 2.

96. A flagellin variant that has the amino acid sequence of SEQ ID NO: 2.

97. The flagellin variant of any one of claims 87-94, wherein the amino acid sequence of SEQ ID NO: 2 does not comprise the terminal histidine tag sequence of SEQ ID NO: 5.

98. A method of treating cancer comprising administering a flagellin variant in conjunction with a checkpoint inhibitor to a subject in need thereof.

99. The method of claim 98, wherein the flagellin variant is selected from entolimod (SEQ ID NO: 3), 33MX (SEQ ID NO: 1), and SE-2 (SEQ ID NO: 2).

100. The method of claim 98 or 99, wherein the checkpoint inhibitor is selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

101. The method of any one of claims 98-100, wherein the flagellin variant is administered prior to administration of the checkpoint inhibitor.

102. The method of any one of claims 98-100, wherein the flagellin variant is administered at the same time as administration of the checkpoint inhibitor.

103. The method of any one of claims 98-100, wherein the flagellin variant is administered after administration of the checkpoint inhibitor.


SEQUENCE LISTING (40 sequences)

SEQ ID NO: 1 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 2 - protein, 239 aa, Artificial Sequence
  Synthetic polypeptide.
MSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQLVPRGSHHHHHHG

SEQ ID NO: 3 - protein, 329 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 4 - protein, 505 aa, Artificial Sequence
  Synthetic polypeptide.
MAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAAAPDKVYVNAANGQLTTDDAENNTAVDLFKTTKSTAGTAEAKAIAGAIKGGKEGDTFDYKGVTFTIDTKTGDDGNGKVSTTINGEKVTLTVADIATGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 5 - protein, 6 aa, Artificial Sequence
  Synthetic polypeptide.
HHHHHH

SEQ ID NO: 6 - protein, 239 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQLVPRGSHHHHHHG

SEQ ID NO: 7 - protein, 250 aa, Artificial Sequence
  Synthetic polypeptide.
MSGLRINSAKDDAAGQAIANRADSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 8 - protein, 250 aa, Artificial Sequence
  Synthetic polypeptide.
MSGLRINSAKDDAAGQAIANRSDSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 9 - protein, 250 aa, Artificial Sequence
  Synthetic polypeptide.
MSGLRINSAKDDAAGQAIANRTDSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 10 - protein, 250 aa, Artificial Sequence
  Synthetic polypeptide.
MSGLRINSAKDDAAGQAIANRFDDNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 11 - protein, 250 aa, Artificial Sequence
  Synthetic polypeptide.
MSGLRINSAKDDAAGQAAANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 12 - protein, 250 aa, Artificial Sequence
  Synthetic polypeptide.
MSGLRINSAKDDAAGQAIANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 13 - protein, 250 aa, Artificial Sequence
  Synthetic polypeptide.
MSGLRINSAKDDAAGQAIANRFDSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 14 - protein, 250 aa, Artificial Sequence
  Synthetic polypeptide.
MSGLRINSAKDDAAGQAEANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 15 - protein, 250 aa, Artificial Sequence
  Synthetic polypeptide.
MSGLRINSAKDDAAGQATANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 16 - protein, 250 aa, Artificial Sequence
  Synthetic polypeptide.
MSGLRINSAKDDAAGQAIANRFTSNIEGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 17 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQADQQAGTSTLAQANQVPQNVLSLLVPRGSHHHHHHG

SEQ ID NO: 18 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQADQQAGTSVDAQANQVPQNVLSLLVPRGSHHHHHHG

SEQ ID NO: 19 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQANQQAGTSTLAQANQVPQNVLSLLVPRGSHHHHHHG

SEQ ID NO: 20 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQANQQAGTSVDAQANQVPQNVLSLLVPRGSHHHHHHG

SEQ ID NO: 21 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQASQQAGTSTLAQANQVPQNVLSLLVPRGSHHHHHHG

SEQ ID NO: 22 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQASQQAGTSVDAQANQVPQNVLSLLVPRGSHHHHHHG

SEQ ID NO: 23 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQALQQAGTSVLAQANQVPQNVLSLLVPRGSHHHHHHG

SEQ ID NO: 24 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQIAQQAGTSVLAQANQVPQNVLSLLVPRGSHHHHHHG

SEQ ID NO: 25 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSTLAQANQVPQNVLSLLVPRGSHHHHHHG

SEQ ID NO: 26 - protein, 245 aa, Artificial Sequence
  Synthetic polypeptide.
MHHHHHHSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQIDQQAGTSVLAQANQVPQNVLSLLR

SEQ ID NO: 27 - protein, 245 aa, Artificial Sequence
  Synthetic polypeptide.
MHHHHHHSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQIHQQAGTSVLAQANQVPQNVLSLLR

SEQ ID NO: 28 - protein, 245 aa, Artificial Sequence
  Synthetic polypeptide.
MHHHHHHSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSVLAQANQVPQNVLSLLR

SEQ ID NO: 29 - protein, 245 aa, Artificial Sequence
  Synthetic polypeptide.
MHHHHHHSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGDSVLAQANQVPQNVLSLLR

SEQ ID NO: 30 - protein, 245 aa, Artificial Sequence
  Synthetic polypeptide.
MHHHHHHSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQKDQQAGTSVLAQANQVPQNVLSLLR

SEQ ID NO: 31 - protein, 245 aa, Artificial Sequence
  Synthetic polypeptide.
MHHHHHHSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGDSVLAQANQVPQNVLSLLR

SEQ ID NO: 32 - protein, 233 aa, Artificial Sequence
  Synthetic polypeptide.
MHHHHHHSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGDSVLAQG

SEQ ID NO: 33 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNASSLLVPRGSHHHHHHG

SEQ ID NO: 34 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNTASLLVPRGSHHHHHHG

SEQ ID NO: 35 - protein, 245 aa, Artificial Sequence
  Synthetic polypeptide.
MHHHHHHSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQANQVPQNVLSLLR

SEQ ID NO: 36 - protein, 250 aa, Artificial Sequence
  Synthetic polypeptide.
MHHHHHHLVPRGSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQANQVPQNVLSLLR

SEQ ID NO: 37 - protein, 251 aa, Artificial Sequence
  Synthetic polypeptide.
MLVPRGSHHHHHHSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQANQVPQNVLSLLR

SEQ ID NO: 38 - protein, 250 aa, Artificial Sequence
  Synthetic polypeptide.
MHHHHHHGSPRGSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQANQVPQNVLSLLR

SEQ ID NO: 39 - protein, 250 aa, Artificial Sequence
  Synthetic polypeptide.
MHHHHHHAVPRGSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQANQVPQNVLSLLR

SEQ ID NO: 40 - protein, 250 aa, Artificial Sequence
  Synthetic polypeptide.
MHHHHHHAAPRGSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQANQVPQNVLSLLR